(19) United States
(12) Patent Application Publication
(10) Pub. No.: US 2021/0340205 A1
(43) Pub. Date: Nov. 4, 2021

(54) GFRAL EXTRACELLULAR DOMAINS AND METHODS OF USE

(71) Applicant: NOVARTIS AG, Basel (CH)

(72) Inventors: Jirong BAI, Dedham, MA (US); Kurt Alex HELDWEIN, Belmont, MA (US); Loren Daniel LINDSLEY, Boston, MA (US)

(21) Appl. No.: 17/266,828

(22) PCT Filed: Aug. 9, 2019
(86) PCT No.: PCT/IB2019/056803

§ 371 Date: Feb. 8, 2021

Related U.S. Application Data
(60) Provisional application No. 62/717,052, filed on Aug. 10, 2018.

(51) Int. Cl. C07K 14/495; G01N 33/68; G01N 33/566; C07K 16/22; C07K 16/28; C07K 14/71

(52) U.S. Cl. CPC C07K 14/495; G01N 33/6863; G01N 33/566; C07K 16/22; C07K 16/2863; C07K 14/71; A61K 38/00; C07K 2319/41; C07K 2319/21; C07K 2319/43; C07K 2319/91; C07K 2319/02; C07K 2319/03; G01N 2333/495

(57) ABSTRACT

GFRAL extracellular domains comprising domains D2 and D3 are disclosed. The disclosure further relates to methods and compositions for screening and evaluating the activity of a GFRAL ligand, such as a GDF15 peptide, using the GFRAL extracellular domains provided herein. Also disclosed are methods and compositions for treating obesity, reducing appetite, and/or reducing body weight using the GFRAL extracellular domains provided herein.

The present disclosure relates to GFRAL extracellular domains, as well as methods and compositions for using the GFRAL extracellular domains provided herein. The present disclosure further relates to cell-based assays for screening and evaluating the activity of a GFRAL ligand (e.g., a GDF15 peptide), as well as methods of treatment using the GFRAL extracellular domains and GFRAL ligands. Cells and kits useful for screening and evaluating the activity of a GFRAL ligand (e.g., a GDF15 peptide) are also provided.

### BACKGROUND OF THE INVENTION

Growth/differentiation factor 15 (GDF15) is a divergent member of the transforming growth factor-β (TGF-β) cytokine super family that has been implicated in various biological functions, including cancer cachexia, renal and heart failure, atherosclerosis, and metabolism (Breit et al., Growth Factors 2011; 29(5):187-95). A connection between GDF15 and body weight regulation was initially suggested based on an observation that increasing GDF15 levels in serum correlated with weight loss in individuals with advanced prostate cancer (Johnen et al., Nat Med 2007; 13(11):1333-40). In mice with xenografted prostate tumors, elevated GDF15 levels have also been associated with marked weight, fat, and lean tissue loss mediated by decreased food intake and capable of being reversed by administration of an antibody to GDF15 (Johnen et al., Nat Med 2007; 13(11):1333-40). Additionally, long-term elevated expression of GDF15 in mice has been shown to result in decreased food intake, body weight, and adiposity with concomitantly improved glucose tolerance, both under normal and obesogenic dietary conditions (Macia et al., PLoS One 2012; 7(4):e34868). The metabolic actions of GDF15 with respect to appetite and body weight make it a promising therapy for patients suffering from obesity and/or related comorbidities.

GFRAL, an orphan member of the glial cell line-derived neurotrophic factor (GDNF) receptor alpha family, is a high-affinity receptor for GDF15. GFRAL may also be necessary for the appetite suppressing effect of GDF15. GDF15-mediated reductions in food intake and body weight of mice with obesity were abolished in GFRAL knockout mice (Yang et al., Nat Med 2017; 23(10):1158-66). GFRAL requires association with the co-receptor RET to elicit intracellular signaling in response to GDF15 stimulation (Yang et al., Nat Med 2017; 23(10):1158-66).

Recombinant GDF15 proteins as potential therapeutic agents have been reported and more are under investigation (Xiong et al., Sci Transl Med 2017; 9(412):eaan8732). Thus, an assay for quickly and effectively evaluating the activity of such therapeutic proteins would be a desirable screening tool. Assays related to GDF15 and GFRAL have been described in WO 2017/121865, WO 2017/152105 and WO 2018/071493. Likewise, novel methods and compositions for improving the activity of therapeutic GDF15 compositions would also be beneficial.

### SUMMARY OF THE INVENTION

The present disclosure provides, in various embodiments, novel methods and assays for detecting and testing the activity of a GFRAL ligand (e.g., GDF15, e.g., a GDF15 peptide). Also disclosed are methods and compositions for treating obesity and related disorders using a GFRAL ligand (e.g., a GDF15 peptide screened for activity according to the methods disclosed herein) alone or in combination with a soluble GFRAL (e.g., one comprising the D2 and D3 extracellular domains but not the D1 extracellular domain).

In various embodiments, the present disclosure more specifically relates to cell-based methods and assays for evaluating the activity of a GFRAL ligand. Cell-based potency assays are often the preferred format for determining the biological activity of a biological product, since they can measure the biological response elicited by the product and can generate results within a relatively short period of time, as compared to animal-based assays. In addition, many cell-based potency assays have defined correlation with the product's mechanism of action. Thus, such assays are widely used and often provided to drug administration authorities for drug registration and pre-market approval.

In various embodiments, the cell-based methods and assays disclosed herein are cell-based signal transduction assays and may be useful for determining the GFRAL signaling activity of a GFRAL ligand (e.g., a GDF15 peptide). In various embodiments, the present disclosure provides a method of detecting the activity of a GDF15 peptide, comprising: (a) providing a cell that expresses a cell surface receptor kinase; (b) contacting the cell with the GDF15 peptide and a soluble GFRAL; and (c) detecting a biological response in the contacted cell, wherein the soluble GFRAL comprises a GFRAL extracellular domain comprising domains D2 and D3.

In some embodiments, the soluble GFRAL comprises a GFRAL extracellular domain lacking domain D1. In some embodiments, the soluble GFRAL further comprises a signal peptide. In some embodiments, the soluble GFRAL comprises the amino acid sequence of SEQ ID NO:1 or a functional variant thereof. In some embodiments, the soluble GFRAL or functional variant has at least 80% amino acid sequence identity with the amino acid sequence of SEQ ID NO:1. In some embodiments, the soluble GFRAL or functional variant has at least 90% amino acid sequence identity with the amino acid sequence of SEQ ID NO:1. In some embodiments, the soluble GFRAL comprises the amino acid sequence of SEQ ID NO:2 or a functional variant thereof. In some embodiments, the soluble GFRAL or functional variant thereof, e.g., comprising the amino acid sequence of SEQ ID NO:1 or SEQ ID NO:2, further comprises (e.g., is fused to) an affinity tag. In some embodiments, the affinity tag comprises an amyloid-beta precursor protein (App) tag, a histidine (His) tag, a FLAG tag, or a myc tag, or a combination thereof. In some embodiments, the soluble GFRAL comprises the amino acid sequence of SEQ ID NO:3 or a functional variant thereof, or of SEQ ID NO:25 or a functional variant thereof.

In some embodiments, the GDF15 peptide comprises the amino acid sequence of SEQ ID NO:13 or a functional variant thereof, including amino acid sequences of SEQ ID NO: 14, 15, 16 or 17. In some embodiments, the GDF15 peptide or functional variant has at least 80% amino acid sequence identity with the amino acid sequence of SEQ ID NO:13. In some embodiments, the GDF15 peptide or functional variant has at least 90% amino acid sequence identity with the amino acid sequence of SEQ ID NO:13. In some embodiments, the GDF15 peptide comprises an affinity tag, a fusion, a conjugation, a PEGylation, and/or a glycosylation. In some embodiments, the GDF15 peptide is tagged with an amyloid-beta precursor protein (App) tag, a histidine (His) tag, a FLAG tag, or a myc tag, or a combination thereof. In some embodiments, the GDF15 peptide is fused to a human serum albumin, a mouse serum albumin, an immunoglobulin constant region, or an alpha-1-antitrypsin. In other embodiments, the GDF15 peptide is conjugated to a fatty acid.

In some embodiments of the cell-based methods and assays, the cell is contacted with the GDF15 peptide and the soluble GFRAL simultaneously. In some other embodiments, the cell is contacted with the GDF15 peptide and the soluble GFRAL sequentially. In some embodiments, the GDF15 peptide and the soluble GFRAL are in the same composition. In some embodiments, the GDF15 peptide and the soluble GFRAL are in a mixture. In some embodiments, the GDF15 peptide and the soluble GFRAL are in a complex. In some embodiments, the GDF15 peptide and the soluble GFRAL are in a binary complex.

In some embodiments of the cell-based methods and assays, the cell surface receptor kinase is an endogenous cell surface receptor kinase. In some other embodiments, the cell surface receptor kinase is an exogenous cell surface receptor kinase. In some embodiments, the cell surface receptor kinase is a RET receptor tyrosine kinase.

In some embodiments of the cell-based methods and assays, the cell does not express endogenous GFRAL. In some embodiments of the cell-based methods and assays, the cell does not express partial or full length GFRAL (e.g., a human GFRAL comprising a transmembrane domain and further a cytoplasmic domain). In some embodiments, the cell does not express endogenous GDF15. In some embodiments, the cell is a GDF15 knockout (KO) cell comprising an inoperative GDF15 gene. In some embodiments, the cell is a mammalian cell. In some embodiments, the cell is a human cell. In some embodiments, the cell is an MCF7 cell (e.g., ATCC® HTB-22™), a breast cancer cell (see, e.g., Comsa et al., Anticancer Res 2015; 35(6):3147-54). In some embodiments, the cell is an SH-SY5Y cell (e.g., ATCC® CRL-2266™), a bone marrow neuroblastoma cell. In still other embodiments, the cell is an HEK293A-GDF15 knockout (KO) cell. Other exemplary cell types are also described herein.

In some embodiments, a biological response is induced when a GDF15 peptide, a soluble GFRAL, and a cell surface receptor kinase, e.g., RET, form a ternary complex. In some embodiments, the biological response is not induced in a cell contacted with the GDF15 peptide in the absence of the soluble GFRAL. In some embodiments, the biological response is a signal transduction response (e.g., a signal transduction response downstream of GDF15, for example, ERK or AKT signaling).

The biological response, in some embodiments, is an increase or decrease in the expression or activity of a protein in the cell, as compared to the expression or activity of the same protein in a control cell that is not contacted with the GDF15 peptide and/or the soluble GFRAL. In some embodiments, the biological response is an increase or decrease in the expression or activity of an intracellular protein in one or more of the RET-ERK, RET-AKT, protein kinase C, JAK/STAT, JNK, p38, and RAC1 pathways. In some embodiments, the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR. In some embodiments, the biological response is detected using one or more assays selected from a kinase or enzymatic activity assay, incubation of whole cells with radiolabeled ³²P-orthophosphate, two-dimensional gel electrophoresis, an immunoblot assay (e.g., Western blot), an AlphaLISA® assay, an enzyme-linked immunosorbent assay (ELISA), a cell-based ELISA assay, intracellular flow cytometry, immunocytochemistry (ICC), immunohistochemistry (IHC), mass spectrometry, multi-analyte profiling (e.g., a phospho-protein multiplex assay), and fluorescent in situ hybridization (FISH).

In some embodiments, the cell surface receptor kinase is a RET receptor tyrosine kinase and the protein is an intracellular protein in the RET-ERK pathway. In some embodiments, the intracellular protein is selected from one or more of ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2, or any downstream targets thereof. In some embodiments, the intracellular protein is selected from one or more of ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the ERK is ERK1 or ERK2. In some embodiments, the ERK is ERK1 (also referred to as MAPK3 or PRKM3). In some embodiments, the ERK is ERK2 (also referred to as MAPK1, PRKM1, or PRKM2).

In other embodiments, the cell surface receptor kinase is a RET receptor tyrosine kinase and the protein is an intracellular protein in the RET-AKT pathway. In some embodiments, the intracellular protein is selected from one or more of AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR, or any downstream targets thereof. In some embodiments, the intracellular protein is selected from one or more of AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR. Exemplary downstream targets include, but are not limited to, S6 kinase. In some embodiments, the AKT (also referred to as PKB or RAC) is AKT1, AKT2, or AKT3. In some embodiments, the RAS is H-RAS, K-RAS, or N-RAS.

The biological response, in some other embodiments, is an increase or decrease in phosphorylation of a protein kinase in the cell, as compared to phosphorylation of the same protein kinase in a control cell that is not contacted with the GDF15 peptide and the soluble GFRAL.

In some embodiments, the protein kinase is the cell surface receptor kinase. In some embodiments, the protein kinase and/or cell surface receptor kinase is a RET receptor tyrosine kinase. In some other embodiments, the protein kinase is an intracellular protein kinase, wherein the intracellular protein kinase is directly or indirectly phosphorylated by the cell surface receptor kinase.

In some embodiments, the protein kinase is an intracellular protein kinase in the RET-ERK pathway. In some embodiments, the intracellular protein kinase is selected from one or more of ERK, JAK1, JAK2, RAF, MEK1, MEK2, RSK1, RSK2, RSK3, MNK1, MNK2, MSK1, and MSK2, or any downstream targets thereof. In some embodiments, the intracellular protein kinase is selected from one or more of ERK, JAK1, JAK2, RAF, MEK1, MEK2, RSK1, RSK2, RSK3, MNK1, MNK2, MSK1, and MSK2. In some embodiments, the intracellular protein kinase is ERK (e.g., ERK1 or ERK2). In some embodiments, the intracellular protein kinase is ERK1 and/or ERK2.

In some other embodiments, the protein kinase is an intracellular protein kinase in the RET-AKT pathway. In some embodiments, the intracellular protein kinase is selected from one or more of AKT, SRC, JAK1, JAK2, PI3K, PDK1, MLK3, ASK1, GSK3alpha, GSK3beta, and mTOR, or any downstream targets thereof. In some embodiments, the intracellular protein kinase is selected from one or more of AKT, SRC, JAK1, JAK2, PI3K, PDK1, MLK3, ASK1, GSK3alpha, GSK3beta, and mTOR. In some embodiments, the intracellular protein kinase is AKT (e.g., AKT1, AKT2, or AKT3). In some embodiments, the intracellular protein kinase is AKT1, AKT2, and/or AKT3. In some embodiments, the downstream target in the RET-AKT pathway is S6 kinase.

In various other embodiments, the present disclosure provides a method of detecting the activity of a GDF15 peptide, comprising: (a) providing a cell that expresses a GFRAL extracellular domain (e.g., a soluble GFRAL extracellular domain) and a cell surface receptor kinase; (b) contacting the cell with the GDF15 peptide; and (c) detecting a biological response in the contacted cell, wherein the GFRAL extracellular domain comprises domains D2 and D3.

In various other embodiments, the present disclosure provides a method of detecting the activity of a GDF15 peptide, comprising: (a) providing a cell that expresses a GFRAL extracellular domain (e.g., a soluble GFRAL extracellular domain) and a cell surface receptor kinase; (b) contacting the cell with the GDF15 peptide; and (c) detecting a biological response in the contacted cell, wherein the GFRAL extracellular domain comprises domains D2 and D3; and wherein the cell does not endogenously express GFRAL.

In various embodiments, the biological response in the contacted cell is a response related to cell signaling or signal transduction (e.g., phosphorylation of a protein kinase). In various embodiments, the biological response is detected using one or more assays selected from a kinase or enzymatic activity assay, incubation of whole cells with radiolabeled ³²P-orthophosphate, two-dimensional gel electrophoresis, an immunoblot assay (e.g., Western blot), an AlphaLISA® assay, an enzyme-linked immunosorbent assay (ELISA), a cell-based ELISA assay, intracellular flow cytometry, immunocytochemistry (ICC), immunohistochemistry (IHC), mass spectrometry, multi-analyte profiling (e.g., a phospho-protein multiplex assay), and fluorescent in situ hybridization (FISH). Other exemplary biological responses include but are not limited to cellular responses related to gene transcription, protein expression, toxicity, cytokine release, cell proliferation, cell motility or morphology, cell growth arrest, or cell death (e.g., apoptosis).

In some embodiments, the GFRAL extracellular domain lacks domain D1. In some embodiments, the GFRAL extracellular domain is a soluble GFRAL extracellular domain. In some embodiments, the GFRAL extracellular domain is attached to the cell surface by a tether. In some embodiments, the tether is a GFRAL transmembrane domain or a functional fragment thereof. In some embodiments, the GFRAL extracellular domain or tether comprises the amino acid sequence of a native GFRAL transmembrane domain. In some embodiments, the GFRAL extracellular domain or tether comprises the amino acid sequence of SEQ ID NO:18 or a functional variant thereof. In some embodiments, the tether is a heterologous transmembrane domain fused to the GFRAL extracellular domain.

In some embodiments, the tether is a glycophosphatidylinositol (GPI) or a sequence capable of directing GPI linker addition. In some embodiments, the GFRAL extracellular domain or tether comprises the amino acid sequence of SEQ ID NO:19 or a functional variant thereof, SEQ ID NO:20 or a functional variant thereof, or SEQ ID NO:21 or a functional variant thereof. In some embodiments, the tether is a membrane-inserting sequence. In some embodiments, the GFRAL extracellular domain or tether comprises the amino acid sequence of SEQ ID NO:22 or a functional variant thereof, or SEQ ID NO:23 or a functional variant thereof. In some other embodiments, the tether is a membrane-inserting fatty acid.

In some embodiments, the GFRAL extracellular domain further comprises a signal peptide. In some embodiments, the GFRAL extracellular domain comprises or consists of the amino acid sequence of SEQ ID NO:1 or a functional variant thereof. In some embodiments, the GFRAL extracellular domain or functional variant has at least 80% amino acid sequence identity with the amino acid sequence of SEQ ID NO:1. In some embodiments, the GFRAL extracellular domain or functional variant has at least 90% amino acid sequence identity with the amino acid sequence of SEQ ID NO:1. In some embodiments, the GFRAL extracellular domain or functional variant has at least 95% amino acid sequence identity with the amino acid sequence of SEQ ID NO:1. In some embodiments, the GFRAL extracellular domain comprises or consists of the amino acid sequence of SEQ ID NO:2 or a functional variant thereof. In some embodiments, the GFRAL extracellular domain, e.g., comprising the amino acid sequence of SEQ ID NO:1 or SEQ ID NO:2, further comprises (e.g., is fused to) an affinity tag. In some embodiments, the affinity tag comprises an amyloid-beta precursor protein (App) tag, a histidine (His) tag, a FLAG tag, or a myc tag, or a combination thereof. In some embodiments, the GFRAL extracellular domain comprises the amino acid sequence of SEQ ID NO:3 or a functional variant thereof, or of SEQ ID NO:25 or a functional variant thereof.

In some embodiments, the GDF15 peptide comprises or consists of the amino acid sequence of SEQ ID NO:13 or a functional variant thereof, including amino acid sequences of SEQ ID NO: 14, 15, 16 or 17. In some embodiments, the GDF15 peptide or functional variant has at least 80% amino acid sequence identity with the amino acid sequence of SEQ ID NO:13. In some embodiments, the GDF15 peptide or functional variant has at least 90% amino acid sequence identity with the amino acid sequence of SEQ ID NO:13. In some embodiments, the GDF15 peptide or functional variant has at least 95% amino acid sequence identity with the amino acid sequence of SEQ ID NO:13. In some embodiments, the GDF15 peptide further comprises (e.g., is fused to) an affinity tag, a fusion, a conjugation, a PEGylation, and/or a glycosylation. In some embodiments, the GDF15 peptide is tagged with an amyloid-beta precursor protein (App) tag, a histidine (His) tag, a FLAG tag, or a myc tag, or a combination thereof. In some embodiments, the GDF15 peptide is fused to a human serum albumin, a mouse serum albumin, an immunoglobulin constant region, or an alpha-1-antitrypsin. In other embodiments, the GDF15 peptide is conjugated to a fatty acid.

In some embodiments, the cell surface receptor kinase is an endogenous cell surface receptor kinase. In some other embodiments, the cell surface receptor kinase is an exogenous cell surface receptor kinase. In some embodiments, the cell surface receptor kinase is a RET receptor tyrosine kinase.

In some embodiments, the cell does not express endogenous GFRAL. In some embodiments, the cell does not express full length GFRAL. In some embodiments, the cell does not express endogenous GDF15. In some embodiments, the cell is a GDF15 KO cell comprising an inoperative GDF15 gene. In some embodiments, the cell is a mammalian cell. In some embodiments, the cell is a human cell. In some embodiments, the cell is an MCF7 cell. In some embodiments, the cell is an SH-SY5Y cell. In still other embodiments, the cell is an HEK293A-GDF15 KO cell.

In some embodiments, a biological response is induced when a GDF15 peptide, a GFRAL extracellular domain, and a cell surface receptor kinase (e.g., RET) form a ternary complex. In some embodiments, the biological response is a signal transduction response (e.g., a signal transduction response downstream of GDF15, for example, ERK or AKT signaling). In some embodiments, the biological response is an increase or decrease in the expression or activity of a protein in the cell, as compared to the expression or activity of the same protein in a control cell that is not contacted with the GDF15 peptide and the soluble GFRAL. The protein having increased or decreased expression or activity can be any of the exemplary proteins described herein, e.g., an intracellular protein in one or more of the RET-ERK, RET-AKT, protein kinase C, JAK/STAT, JNK, p38, and RAC1 pathways. In some embodiments, the biological response is detected using any of the exemplary assays disclosed herein.

In some embodiments, the biological response is an increase or decrease in the expression or activity of an intracellular protein in one or more of the RET-ERK, RET-AKT, protein kinase C, JAK/STAT, JNK, p38, and RAC1 pathways. In some embodiments, the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

In some embodiments, the cell surface receptor kinase is a RET receptor tyrosine kinase and the protein is an intracellular protein in the RET-ERK pathway. In some embodiments, the intracellular protein is selected from one or more of ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2, or any downstream targets thereof. In some embodiments, the intracellular protein is selected from one or more of ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the ERK is ERK1 or ERK2.

In other embodiments, the cell surface receptor kinase is a RET receptor tyrosine kinase and the protein is an intracellular protein in the RET-AKT pathway. In some embodiments, the intracellular protein is selected from one or more of AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR, or any downstream targets thereof. In some embodiments, the intracellular protein is selected from one or more of AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR. In some embodiments, the AKT is AKT1, AKT2, or AKT3.

The biological response, in some other embodiments, is an increase or decrease in phosphorylation of a protein kinase in the cell, as compared to phosphorylation of the same protein kinase in a control cell that is not contacted with the GDF15 peptide.

In some embodiments, the protein kinase is the cell surface receptor kinase. In some embodiments, the protein kinase and/or cell surface receptor kinase is a RET receptor tyrosine kinase. In some other embodiments, the protein kinase is an intracellular protein kinase, wherein the intracellular protein kinase is directly or indirectly phosphorylated by the cell surface receptor kinase.

In some embodiments, the protein kinase is an intracellular protein kinase in the RET-ERK pathway. In some embodiments, the intracellular protein kinase is selected from one or more of ERK, JAK1, JAK2, RAF, MEK1, MEK2, RSK1, RSK2, RSK3, MNK1, MNK2, MSK1, and MSK2, or any downstream targets thereof. In some embodiments, the intracellular protein kinase is selected from one or more of ERK, JAK1, JAK2, RAF, MEK1, MEK2, RSK1, RSK2, RSK3, MNK1, MNK2, MSK1, and MSK2. In some embodiments, the intracellular protein kinase is ERK (e.g., ERK1 or ERK2). In some embodiments, the intracellular protein kinase is ERK1 and/or ERK2.

In some other embodiments, the protein kinase is an intracellular protein kinase in the RET-AKT pathway. In some embodiments, the intracellular protein kinase is selected from one or more of AKT, SRC, JAK1, JAK2, PI3K, PDK1, MLK3, ASK1, GSK3alpha, GSK3beta, and mTOR, or any downstream targets thereof. In some embodiments, the intracellular protein kinase is selected from one or more of AKT, SRC, JAK1, JAK2, PI3K, PDK1, MLK3, ASK1, GSK3alpha, GSK3beta, and mTOR. In some embodiments, the intracellular protein kinase is AKT (e.g., AKT1, AKT2, or AKT3). In some embodiments, the intracellular protein kinase is AKT1, AKT2, and/or AKT3. In some embodiments, the downstream target in the RET-AKT pathway is S6 kinase.

Further provided herein, in various embodiments, are isolated and modified cells for detecting the activity of a GDF15 peptide. In various embodiments, the cells express a GFRAL extracellular domain comprising domains D2 and D3 and a cell surface receptor kinase. In various embodiments, the GFRAL extracellular domain comprises domains D2 and D3 but lacks domain D1. In some embodiments, the GFRAL extracellular domain further comprises a signal peptide. In some embodiments, the GFRAL extracellular domain comprises or consists of the amino acid sequence of SEQ ID NO:1 or a functional variant thereof. In some embodiments, the GFRAL extracellular domain or functional variant has at least 80% amino acid sequence identity with the amino acid sequence of SEQ ID NO:1. In some embodiments, the GFRAL extracellular domain or functional variant has at least 90% amino acid sequence identity with the amino acid sequence of SEQ ID NO:1. In some embodiments, the GFRAL extracellular domain comprises or consists of the amino acid sequence of SEQ ID NO:2 or a functional variant thereof. In some embodiments, the GFRAL extracellular domain, e.g., comprising the amino acid sequence of SEQ ID NO:1 or SEQ ID NO:2, further comprises (e.g., is fused to) an affinity tag. In some embodiments, the affinity tag comprises an amyloid-beta precursor protein (App) tag, a histidine (His) tag, a FLAG tag, or a myc tag, or a combination thereof. In some embodiments, the GFRAL extracellular domain comprises the amino acid sequence of SEQ ID NO:3 or a functional variant thereof, or of SEQ ID NO:25 or a functional variant thereof.

In some embodiments, the GDF15 peptide comprises the amino acid sequence of SEQ ID NO:13 or a functional variant thereof, including amino acid sequences of SEQ ID NO: 14, 15, 16 or 17. In some embodiments, the GDF15 peptide or functional variant has at least 80% amino acid sequence identity with the amino acid sequence of SEQ ID NO:13. In some embodiments, the GDF15 peptide or functional variant has at least 90% amino acid sequence identity with the amino acid sequence of SEQ ID NO:13. In some embodiments, the GDF15 peptide comprises an affinity tag, a fusion, a conjugation, a PEGylation, and/or a glycosylation. In some embodiments, the GDF15 peptide is tagged with an amyloid-beta precursor protein (App) tag, a histidine (His) tag, a FLAG tag, or a myc tag, or a combination thereof. In some embodiments, the GDF15 peptide is fused to a human serum albumin, a mouse serum albumin, an immunoglobulin constant region, or an alpha-1-antitrypsin. In other embodiments, the GDF15 peptide is conjugated to a fatty acid.

In some embodiments, the cell surface receptor kinase is an endogenous cell surface receptor kinase. In some other embodiments, the cell surface receptor kinase is an exogenous cell surface receptor kinase. In some embodiments, the cell surface receptor kinase is a RET receptor tyrosine kinase.

In some embodiments, the cell does not express endogenous GFRAL. In some embodiments, the cell does not express full length GFRAL. In some embodiments, the cell does not express endogenous GDF15. In some embodiments, the cell is a GDF15 KO cell comprising an inoperative GDF15 gene. In some embodiments, the cell is a mammalian cell. In some embodiments, the cell is a human cell. In some other embodiments, the cell is an MCF7 cell. In some embodiments, the cell is an SH-SY5Y cell. In still other embodiments, the cell is an HEK293A-GDF15 KO cell.

Further provided herein, in various embodiments, are kits for determining the activity of a GDF15 peptide. In various embodiments, the kits comprise a cell for contacting with the GDF15 peptide, and a means of detecting a biological response in the contacted cell. In various embodiments, the cell is an isolated modified cell that expresses a GFRAL extracellular domain comprising domains D2 and D3 and a cell surface receptor kinase

Also provided herein, in various embodiments, are therapeutic methods and uses for the GFRAL ligands (e.g., GDF15 peptides) and GFRAL extracellular domains disclosed herein, e.g., in treating obesity or obesity-related disorders, in reducing appetite, and/or in reducing body weight in a subject, etc. In various embodiments, the therapeutic methods and uses described herein are useful in treating obesity or obesity-related disorders, such as cancers, body weight disorders, and/or metabolic diseases and disorders. Exemplary obesity-related disorders and conditions that can coincide with obesity or may be a direct or indirect result of having excess body weight are disclosed herein. These include but are not limited to cancer, type II diabetes mellitus (T2DM), nonalcoholic steatohepatitis (NASH), hypertriglyceridemia, and cardiovascular disease.

For instance, in certain aspects, the present disclosure provides a method of treating obesity or an obesity-related disorder by administering a GDF15 peptide to a subject, wherein the GDF15 peptide is one that induces a biological response in a cell contacted with the GDF15 peptide and/or has GFRAL signaling activity, e.g., as determined using the detection methods described herein. In some embodiments, the biological response is a signal transduction response (e.g., ERK activation or signaling, or AKT activation or signaling). In some embodiments, the biological response is an increase or decrease in the expression or activity of a protein in the cell, as compared to the expression or activity of the same protein in a control cell that is not contacted with the GDF15 peptide. In some embodiments, the biological response is an increase or decrease in the expression or activity of an intracellular protein in one or more of the RET-ERK, RET-AKT, protein kinase C, JAK/STAT, JNK, p38, and RAC1 pathways. In some embodiments, the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

In some embodiments, the biological response is an increase or decrease in phosphorylation of a protein kinase in the cell, as compared to phosphorylation of the same protein kinase in a control cell that is not contacted with the GDF15 peptide. In some embodiments, the protein kinase is an intracellular protein kinase, wherein the intracellular protein kinase is directly or indirectly phosphorylated by the cell surface receptor kinase. In some embodiments, the subject is overweight or obese. In some embodiments, the subject has a body mass index between 25 and 29.9. In some other embodiments, the subject has a body mass index of 30 or higher. In some embodiments, the obesity-related disorder is a cancer, a body weight disorder, or a metabolic disease or disorder. In some embodiments, the obesity-related disorder is a cancer, T2DM, NASH, hypertriglyceridemia, or cardiovascular disease.

In certain other aspects, the present disclosure provides a use of a GDF15 peptide in treating obesity or an obesity-related disorder in a subject, wherein the GDF15 peptide is one that induces a biological response in a cell contacted with the GDF15 peptide and/or has GFRAL signaling activity, e.g., as determined using the detection methods described herein. In some embodiments, the biological response is a signal transduction response (e.g., ERK activation or signaling, or AKT activation or signaling). In some embodiments, the biological response is an increase or decrease in the expression or activity of a protein in the cell, as compared to the expression or activity of the same protein in a control cell that is not contacted with the GDF15 peptide. In some embodiments, the biological response is an increase or decrease in the expression or activity of an intracellular protein in one or more of the RET-ERK, RET-AKT, protein kinase C, JAK/STAT, JNK, p38, and RAC1 pathways. In some embodiments, the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

In some embodiments, the biological response is an increase or decrease in phosphorylation of a protein kinase in the cell, as compared to phosphorylation of the same protein kinase in a control cell that is not contacted with the GDF15 peptide. In some embodiments, the protein kinase is an intracellular protein kinase, wherein the intracellular protein kinase is directly or indirectly phosphorylated by the cell surface receptor kinase. In some embodiments, the subject is overweight or obese. In some embodiments, the subject has a body mass index between 25 and 29.9. In some other embodiments, the subject has a body mass index of 30 or higher. In some embodiments, the obesity-related disorder is a cancer, a body weight disorder, or a metabolic disease or disorder. In some embodiments, the obesity-related disorder is a cancer, T2DM, NASH, hypertriglyceridemia, or cardiovascular disease.

In certain other aspects, the present disclosure provides a method of reducing appetite and/or body weight by administering a GDF15 peptide to a subject, wherein the GDF15 peptide is one that induces a biological response in a cell contacted with the GDF15 peptide and/or has GFRAL signaling activity, e.g., as determined using the detection methods described herein. In some embodiments, the biological response is a signal transduction response (e.g., ERK activation or signaling, or AKT activation or signaling). In some embodiments, the biological response is an increase or decrease in the expression or activity of a protein in the cell, as compared to the expression or activity of the same protein in a control cell that is not contacted with the GDF15 peptide. In some embodiments, the biological response is an increase or decrease in the expression or activity of an intracellular protein in one or more of the RET-ERK, RET-AKT, protein kinase C, JAK/STAT, JNK, p38, and RAC1 pathways. In some embodiments, the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

In some embodiments, the biological response is an increase or decrease in phosphorylation of a protein kinase in the cell, as compared to phosphorylation of the same protein kinase in a control cell that is not contacted with the GDF15 peptide. In some embodiments, the protein kinase is an intracellular protein kinase, wherein the intracellular protein kinase is directly or indirectly phosphorylated by the cell surface receptor kinase. In some embodiments, the subject is overweight or obese. In some embodiments, the subject has a body mass index between 25 and 29.9. In some other embodiments, the subject has a body mass index of 30 or higher.

In certain other aspects, the present disclosure provides a use of a GDF15 peptide in reducing appetite and/or body weight in a subject, wherein the GDF15 peptide is one that induces a biological response in a cell contacted with the GDF15 peptide and/or has GFRAL signaling activity, e.g., as determined using the detection methods described herein. In some embodiments, the biological response is a signal transduction response (e.g., ERK activation or signaling, or AKT activation or signaling). In some embodiments, the biological response is an increase or decrease in the expression or activity of a protein in the cell, as compared to the expression or activity of the same protein in a control cell that is not contacted with the GDF15 peptide. In some embodiments, the biological response is an increase or decrease in the expression or activity of an intracellular protein in one or more of the RET-ERK, RET-AKT, protein kinase C, JAK/STAT, JNK, p38, and RAC1 pathways. In some embodiments, the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

In some embodiments, the biological response is an increase or decrease in phosphorylation of a protein kinase in the cell, as compared to phosphorylation of the same protein kinase in a control cell that is not contacted with the GDF15 peptide. In some embodiments, the protein kinase is an intracellular protein kinase, wherein the intracellular protein kinase is directly or indirectly phosphorylated by the cell surface receptor kinase. In some embodiments, the subject is overweight or obese. In some embodiments, the subject has a body mass index between 25 and 29.9. In some other embodiments, the subject has a body mass index of 30 or higher.

In certain other aspects, the present disclosure provides a method of treating obesity or an obesity-related disorder, comprising administering a GDF15 peptide and a GFRAL (e.g., a soluble GFRAL) to a subject, wherein the GDF15 peptide induces a biological response in a cell contacted with the GDF15 peptide and/or has GFRAL signaling activity, e.g., as determined using the detection methods described herein, and wherein the GFRAL comprises a GFRAL extracellular domain comprising domains D2 and D3. In some embodiments, the GFRAL is a soluble GFRAL. In some embodiments, the GFRAL comprises a GFRAL extracellular domain lacking domain D1. In some embodiments, the GFRAL further comprises a signal peptide. In some embodiments, the GFRAL consists of a GFRAL extracellular domain lacking domain D1, and optionally a signal peptide. In some embodiments, the GFRAL comprises or consists of the amino acid sequence of SEQ ID NO:1 or a functional variant thereof. In some embodiments, the GFRAL or functional variant has at least 80% amino acid sequence identity with the amino acid sequence of SEQ ID NO:1. In some embodiments, the GFRAL or functional variant has at least 90% amino acid sequence identity with the amino acid sequence of SEQ ID NO:1. In some embodiments, the GFRAL comprises or consists of the amino acid sequence of SEQ ID NO:2 or a functional variant thereof. In some embodiments, the GFRAL further comprises (e.g., is fused to) an affinity tag. In some embodiments, the GFRAL extracellular domain, e.g., comprising the amino acid sequence of SEQ ID NO:1 or SEQ ID NO:2, further comprises (e.g., is fused to) an affinity tag. In some embodiments, the affinity tag comprises an amyloid-beta precursor protein (App) tag, a histidine (His) tag, a FLAG tag, or a myc tag, or a combination thereof. In some embodiments, the GFRAL extracellular domain comprises the amino acid sequence of SEQ ID NO:3 or a functional variant thereof, or of SEQ ID NO:25 or a functional variant thereof. In some embodiments, the GFRAL is a soluble GFRAL comprising a GFRAL extracellular domain comprising domains D2 and D3 but lacking domain D1.

In some embodiments, the GDF15 peptide and the GFRAL are administered simultaneously. In some other embodiments, the GDF15 peptide and the GFRAL are administered sequentially. In some embodiments, the GDF15 peptide and the GFRAL are in the same composition. In some embodiments, the GDF15 peptide and the GFRAL are in a mixture. In some embodiments, the GDF15 peptide and the GFRAL are in a complex. In some embodiments, the GDF15 peptide and the GFRAL are in a binary complex. In some embodiments, the biological response is a signal transduction response (e.g., ERK activation or signaling, or AKT activation or signaling). In some embodiments, the biological response is an increase or decrease in the expression or activity of a protein in the cell, as compared to the expression or activity of the same protein in a control cell that is not contacted with the GDF15 peptide. In some embodiments, the biological response is an increase or decrease in the expression or activity of an intracellular protein in one or more of the RET-ERK, RET-AKT, protein kinase C, JAK/STAT, JNK, p38, and RAC1 pathways. In some embodiments, the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

In some embodiments, the biological response is an increase or decrease in phosphorylation of a protein kinase in the cell, as compared to phosphorylation of the same protein kinase in a control cell that is not contacted with the GDF15 peptide. In some embodiments, the protein kinase is an intracellular protein kinase, wherein the intracellular protein kinase is directly or indirectly phosphorylated by the cell surface receptor kinase. The GDF15 peptide and the GFRAL can be formulated in one or more suitable therapeutic compositions, e.g., comprising a pharmaceutically acceptable carrier or packaged for storage (e.g., lyophilized) prior to reconstitution for administration to a patient. Administration can be by any suitable route, e.g., intravenous, subcutaneous, parenteral, intramuscular, etc. In some embodiments, the subject is overweight or obese. In some embodiments, the subject has a body mass index between 25 and 29.9. In some other embodiments, the subject has a body mass index of 30 or higher. In some embodiments, the obesity-related disorder is a cancer, a body weight disorder, or a metabolic disease or disorder. In some embodiments, the obesity-related disorder is a cancer, T2DM, NASH, hypertriglyceridemia, or cardiovascular disease.

In certain other aspects, the present disclosure provides a use of a GDF15 peptide and a GFRAL (e.g., a soluble GFRAL) in treating obesity or an obesity-related disorder in a subject, wherein the GDF15 peptide induces a biological response in a cell contacted with the GDF15 peptide and/or has GFRAL signaling activity, e.g., as determined using the detection methods described herein, and wherein the GFRAL comprises a GFRAL extracellular domain comprising domains D2 and D3. In some embodiments, the GFRAL is a soluble GFRAL. In some embodiments, the GFRAL comprises a GFRAL extracellular domain lacking domain D1. In some embodiments, the GFRAL further comprises a signal peptide. In some embodiments, the GFRAL comprises or consists of the amino acid sequence of SEQ ID NO:1 or a functional variant thereof. In some embodiments, the GFRAL or functional variant has at least 80% amino acid sequence identity with the amino acid sequence of SEQ ID NO:1. In some embodiments, the GFRAL or functional variant has at least 90% amino acid sequence identity with the amino acid sequence of SEQ ID NO:1. In some embodiments, the GFRAL comprises or consists of the amino acid sequence of SEQ ID NO:2 or a functional variant thereof. In some embodiments, the GFRAL, e.g., comprising the amino acid sequence of SEQ ID NO:1 or SEQ ID NO:2, further comprises (e.g., is fused to) an affinity tag. In some embodiments, the affinity tag comprises an amyloid-beta precursor protein (App) tag, a histidine (His) tag, a FLAG tag, or a myc tag, or a combination thereof. In some embodiments, the GFRAL comprises the amino acid sequence of SEQ ID NO:3 or a functional variant thereof, or of SEQ ID NO:25 or a functional variant thereof. In some embodiments, the GFRAL is a soluble GFRAL comprising a GFRAL extracellular domain comprising domains D2 and D3 but lacking domain D1.

In some embodiments, the GDF15 peptide and the GFRAL are administered simultaneously. In some other embodiments, the GDF15 peptide and the GFRAL are administered sequentially. In some embodiments, the GDF15 peptide and the soluble GFRAL are in the same composition. In some embodiments, the GDF15 peptide and the GFRAL are in a mixture. In some embodiments, the GDF15 peptide and the GFRAL are in a complex. In some embodiments, the GDF15 peptide and the GFRAL are in a binary complex. In some embodiments, the biological response is a signal transduction response (e.g., ERK activation or signaling, or AKT activation or signaling). In some embodiments, the biological response is an increase or decrease in the expression or activity of a protein in the cell, as compared to the expression or activity of the same protein in a control cell that is not contacted with the GDF15 peptide. In some embodiments, the biological response is an increase or decrease in the expression or activity of an intracellular protein in one or more of the RET-ERK, RET-AKT, protein kinase C, JAK/STAT, JNK, p38, and RAC1 pathways. In some embodiments, the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

In some embodiments, the biological response is an increase or decrease in phosphorylation of a protein kinase in the cell, as compared to phosphorylation of the same protein kinase in a control cell that is not contacted with the GDF15 peptide. In some embodiments, the protein kinase is an intracellular protein kinase, wherein the intracellular protein kinase is directly or indirectly phosphorylated by the cell surface receptor kinase. In some embodiments, the subject is overweight or obese. In some embodiments, the subject has a body mass index between 25 and 29.9. In some other embodiments, the subject has a body mass index of 30 or higher. In some embodiments, the obesity-related disorder is a cancer, a body weight disorder, or a metabolic disease or disorder. In some embodiments, the obesity-related disorder is a cancer, T2DM, NASH, hypertriglyceridemia, or cardiovascular disease.

In certain other aspects, the present disclosure provides a method of reducing appetite and/or body weight, comprising administering a GDF15 peptide and a GFRAL (e.g., a soluble GFRAL) to a subject, wherein the GDF15 peptide induces a biological response in a cell contacted with the GDF15 peptide and/or has GFRAL signaling activity, e.g., as determined using the detection methods described herein, and wherein the GFRAL comprises a GFRAL extracellular domain comprising domains D2 and D3. In some embodiments, the GFRAL is a soluble GFRAL. In some embodiments, the GFRAL comprises a GFRAL extracellular domain lacking domain D1. In some embodiments, the GFRAL further comprises a signal peptide. In some embodiments, the GFRAL consists of a GFRAL extracellular domain lacking domain D1, and optionally a signal peptide. In some embodiments, the GFRAL comprises or consists of the amino acid sequence of SEQ ID NO:1 or a functional variant thereof. In some embodiments, the GFRAL or functional variant has at least 80% amino acid sequence identity with the amino acid sequence of SEQ ID NO:1. In some embodiments, the GFRAL or functional variant has at least 90% amino acid sequence identity with the amino acid sequence of SEQ ID NO:1. In some embodiments, the GFRAL comprises or consists of the amino acid sequence of SEQ ID NO:2 or a functional variant thereof. In some embodiments, the GFRAL, e.g., comprising the amino acid sequence of SEQ ID NO:1 or SEQ ID NO:2, further comprises (e.g., is fused to) an affinity tag. In some embodiments, the affinity tag comprises an amyloid-beta precursor protein (App) tag, a histidine (His) tag, a FLAG tag, or a myc tag, or a combination thereof. In some embodiments, the GFRAL comprises the amino acid sequence of SEQ ID NO:3 or a functional variant thereof, or of SEQ ID NO:25 or a functional variant thereof. In some embodiments, the GFRAL is a soluble GFRAL comprising a GFRAL extracellular domain comprising domains D2 and D3 but lacking domain D1.

In some embodiments, the GDF15 peptide and the GFRAL are administered simultaneously. In some other embodiments, the GDF15 peptide and the GFRAL are administered sequentially. In some embodiments, the GDF15 peptide and the GFRAL are in the same composition. In some embodiments, the GDF15 peptide and the GFRAL are in a mixture. In some embodiments, the GDF15 peptide and the GFRAL are in a complex. In some embodiments, the GDF15 peptide and the GFRAL are in a binary complex. In some embodiments, the biological response is a signal transduction response (e.g., ERK activation or signaling, or AKT activation or signaling). In some embodiments, the biological response is an increase or decrease in the expression or activity of a protein in the cell, as compared to the expression or activity of the same protein in a control cell that is not contacted with the GDF15 peptide. In some embodiments, the biological response is an increase or decrease in the expression or activity of an intracellular protein in one or more of the RET-ERK, RET-AKT, protein kinase C, JAK/STAT, JNK, p38, and RAC1 pathways. In some embodiments, the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

In some embodiments, the biological response is an increase or decrease in phosphorylation of a protein kinase in the cell, as compared to phosphorylation of the same protein kinase in a control cell that is not contacted with the GDF15 peptide. In some embodiments, the protein kinase is an intracellular protein kinase, wherein the intracellular protein kinase is directly or indirectly phosphorylated by the cell surface receptor kinase. The GDF15 peptide and the GFRAL can be formulated in one or more suitable therapeutic compositions, e.g., comprising a pharmaceutically acceptable carrier or packaged for storage (e.g., lyophilized) prior to reconstitution for administration to a patient. Administration can be by any suitable route, e.g., intravenous, subcutaneous, parenteral, intramuscular, etc. In some embodiments, the subject is overweight or obese. In some embodiments, the subject has a body mass index between 25 and 29.9. In some other embodiments, the subject has a body mass index of 30 or higher.

In certain other aspects, the present disclosure provides a use of a GDF15 peptide and a GFRAL (e.g., a soluble GFRAL) in reducing appetite and/or body weight in a subject, wherein the GDF15 peptide induces a biological response in a cell contacted with the GDF15 peptide and/or has GFRAL signaling activity, e.g., as determined using the detection methods described herein, and wherein the GFRAL comprises a GFRAL extracellular domain comprising domains D2 and D3. In some embodiments, the GFRAL is a soluble GFRAL. In some embodiments, the GFRAL comprises a GFRAL extracellular domain lacking domain D1. In some embodiments, the GFRAL further comprises a signal peptide. In some embodiments, the GFRAL consists of a GFRAL extracellular domain lacking domain D1, and optionally a signal peptide. In some embodiments, the GFRAL comprises or consists of the amino acid sequence of SEQ ID NO:1 or a functional variant thereof. In some embodiments, the GFRAL or functional variant has at least 80% amino acid sequence identity with the amino acid sequence of SEQ ID NO:1. In some embodiments, the GFRAL or functional variant has at least 90% amino acid sequence identity with the amino acid sequence of SEQ ID NO:1. In some embodiments, the GFRAL comprises or consists of the amino acid sequence of SEQ ID NO:2 or a functional variant thereof. In some embodiments, the GFRAL, e.g., comprising the amino acid sequence of SEQ ID NO:1 or SEQ ID NO:2, further comprises (e.g., is fused to) an affinity tag. In some embodiments, the affinity tag comprises an amyloid-beta precursor protein (App) tag, a histidine (His) tag, a FLAG tag, or a myc tag, or a combination thereof. In some embodiments, the GFRAL comprises the amino acid sequence of SEQ ID NO:3 or a functional variant thereof, or of SEQ ID NO:25 or a functional variant thereof. In some embodiments, the GFRAL is a soluble GFRAL comprising a GFRAL extracellular domain comprising domains D2 and D3 but lacking domain D1.

In some embodiments, the GDF15 peptide and the GFRAL are administered simultaneously. In some other embodiments, the GDF15 peptide and the GFRAL are administered sequentially. In some embodiments, the GDF15 peptide and the soluble GFRAL are in the same composition. In some embodiments, the GDF15 peptide and the GFRAL are in a mixture. In some embodiments, the GDF15 peptide and the GFRAL are in a complex. In some embodiments, the GDF15 peptide and the GFRAL are in a binary complex. In some embodiments, the biological response is a signal transduction response (e.g., ERK activation or signaling, or AKT activation or signaling). In some embodiments, the biological response is an increase or decrease in the expression or activity of a protein in the cell, as compared to the expression or activity of the same protein in a control cell that is not contacted with the GDF15 peptide. In some embodiments, the biological response is an increase or decrease in the expression or activity of an intracellular protein in one or more of the RET-ERK, RET-AKT, protein kinase C, JAK/STAT, JNK, p38, and RAC1 pathways. In some embodiments, the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

In some embodiments, the biological response is an increase or decrease in phosphorylation of a protein kinase in the cell, as compared to phosphorylation of the same protein kinase in a control cell that is not contacted with the GDF15 peptide. In some embodiments, the protein kinase is an intracellular protein kinase, wherein the intracellular protein kinase is directly or indirectly phosphorylated by the cell surface receptor kinase. In some embodiments, the subject is overweight or obese. In some embodiments, the subject has a body mass index between 25 and 29.9. In some other embodiments, the subject has a body mass index of 30 or higher.

In some embodiments of the therapeutic methods and uses described herein, the GDF15 peptide comprises the amino acid sequence of SEQ ID NO:13 or a functional variant thereof, including amino acid sequences of SEQ ID NO: 14, 15, 16 or 17. In some embodiments, the GDF15 peptide or functional variant has at least 80% amino acid sequence identity with the amino acid sequence of SEQ ID NO:13. In some embodiments, the GDF15 peptide or functional variant has at least 90% amino acid sequence identity with the amino acid sequence of SEQ ID NO:13. In some embodiments, the GDF15 peptide comprises an affinity tag, a fusion, a conjugation, a PEGylation, and/or a glycosylation. In some embodiments, the GDF15 peptide is tagged with an amyloid-beta precursor protein (App) tag, a histidine tag, a FLAG tag, or a myc tag, or a combination thereof. In some embodiments, the GDF15 peptide is fused to a human serum albumin, a mouse serum albumin, an immunoglobulin constant region, or an alpha-1-antitrypsin. In other embodiments, the GDF15 peptide is conjugated to a fatty acid.

In still other aspects, the present disclosure provides GFRAL extracellular domains that are capable of binding to a GFRAL ligand (e.g., a GDF15 peptide). The present disclosure more specifically provides, in various embodiments, GFRAL extracellular domains comprising domains D2 and D3. In some embodiments, the GFRAL extracellular domains lack domain D1. In some embodiments, a GFRAL extracellular domain comprises domains D2 and D3 and lacks domain D1. In some embodiments, the GFRAL extracellular domains lacking domain D1 exhibit increased binding activity to GDF15 as compared to GFRAL extracellular domains comprising domain D1. In some embodiments, the GFRAL extracellular domains lacking domain D1 exhibit increased potency for RET activation and/or signaling as compared to GFRAL extracellular domains comprising domain D1, when the GFRAL extracellular domains are bound to or in complex with GDF15.

In some embodiments, the GFRAL extracellular domains are not expressed on the cell surface. In some embodiments, the GFRAL extracellular domains are attached to the cell surface by a tether. In some other embodiments, the GFRAL extracellular domains are soluble.

In various embodiments, the present disclosure also provides a soluble GFRAL comprising a GFRAL extracellular domain comprising domains D2 and D3. In some embodiments, the soluble GFRAL comprises a GFRAL extracellular domain lacking domain D1. In some embodiments, the soluble GFRAL further comprises a signal peptide. In some embodiments, the GFRAL consists of a GFRAL extracellular domain lacking domain D1, and optionally a signal peptide. In some embodiments, the soluble GFRAL comprises or consists of the amino acid sequence of SEQ ID NO:1 or a functional variant thereof. In some embodiments, the soluble GFRAL or functional variant has at least 80% amino acid sequence identity with the amino acid sequence of SEQ ID NO:1. In some embodiments, the soluble GFRAL or functional variant has at least 90% amino acid sequence identity with the amino acid sequence of SEQ ID NO:1. In some embodiments, the soluble GFRAL comprises or consists of the amino acid sequence of SEQ ID NO:2 or a functional variant thereof. In some embodiments, the soluble GFRAL or functional variant thereof, e.g., comprising the amino acid sequence of SEQ ID NO:1 or SEQ ID NO:2, further comprises (e.g., is fused to) an affinity tag. In some embodiments, the affinity tag comprises an amyloid-beta precursor protein (App) tag, a histidine (His) tag, a FLAG tag, or a myc tag, or a combination thereof. In some embodiments, the soluble GFRAL or functional variant thereof comprises the amino acid sequence of SEQ ID NO:3 or a functional variant thereof, or of SEQ ID NO:25 or a functional variant thereof.

In still other aspects, the present disclosure provides methods of identifying agents capable of modulating GDF15 activity, as well as methods of formulating such agents into pharmaceutical compositions.

For instance, in certain aspects, the present disclosure provides a method of identifying an agent capable of modulating GDF15 activity, comprising: (a) contacting an isolated and modified cell with the agent and a GDF15 peptide; and (b) detecting a biological response in the contacted cell, wherein the cell expresses a GFRAL extracellular domain comprising domains D2 and D3 and a cell surface receptor kinase; and wherein the agent is determined to modulate GDF15 activity if the biological response in the contacted cell is increased or decreased relative to the biological response in a cell contacted with the GDF15 peptide in the absence of the agent. In some embodiments, the agent is an antibody. In some embodiments, the agent is an anti-GDF15 antibody. In some embodiments, the agent is an anti-GFRAL antibody. In some embodiments, the GDF15 peptide comprises the amino acid sequence of SEQ ID NO:13 or a functional variant thereof, including amino acid sequences of SEQ ID NO: 14, 15, 16 or 17. In some embodiments, the GDF15 peptide or functional variant has at least 80% amino acid sequence identity with the amino acid sequence of SEQ ID NO:13. In some embodiments, the GDF15 peptide or functional variant has at least 90% amino acid sequence identity with the amino acid sequence of SEQ ID NO:13. In some embodiments, the GDF15 peptide comprises an affinity tag, a fusion, a conjugation, a PEGylation, and/or a glycosylation. In some embodiments, the GDF15 peptide is tagged with an amyloid-beta precursor protein (App) tag, a histidine tag, a FLAG tag, or a myc tag, or a combination thereof. In some embodiments, the GDF15 peptide is fused to a human serum albumin, a mouse serum albumin, an immunoglobulin constant region, or an alpha-1-antitrypsin. In some embodiments, the GDF15 peptide is conjugated to a fatty acid.

In certain aspects, the present disclosure provides a method of identifying an agent capable of modulating GDF15 activity, comprising: (a) providing a cell that expresses a cell surface receptor kinase; (b) contacting the cell with a GDF15 peptide and a soluble GFRAL; (c) contacting the cell with the agent; and (d) detecting a biological response in the contacted cell, wherein the soluble GFRAL comprises a GFRAL extracellular domain comprising domains D2 and D3 and lacks domain D1. In some embodiments, the agent is determined to modulate or increase GDF15 activity if the biological response in the contacted cell is increased in the presence of the GDF15 peptide, the soluble GFRAL, and the agent relative to the biological response in a cell contacted with the GDF15 peptide and the soluble GFRAL in the absence of the agent. In other embodiments, the agent is determined to modulate or decrease GDF15 activity if the biological response in the contacted cell is decreased in the presence of the GDF15 peptide, the soluble GFRAL, and the agent relative to the biological response in a cell contacted with the GDF15 peptide and the soluble GFRAL in the absence of the agent. In some embodiments, the agent is an antibody. In some embodiments, the agent is an anti-GDF15 antibody. In some embodiments, the agent is an anti-GFRAL antibody.

In some embodiments, the soluble GFRAL comprises the amino acid sequence of SEQ ID NO:1 or a functional variant thereof. In some embodiments, the soluble GFRAL or functional variant has at least 80% amino acid sequence identity with the amino acid sequence of SEQ ID NO:1. In some embodiments, the soluble GFRAL or functional variant has at least 90% amino acid sequence identity with the amino acid sequence of SEQ ID NO:1. In some embodiments, the soluble GFRAL, e.g. comprising SEQ ID NO:1 or SEQ ID NO:2, further comprises (e.g., is fused to) an affinity tag. In some embodiments, the affinity tag comprises an amyloid-beta precursor protein (App) tag, a histidine (His) tag, a FLAG tag, or a myc tag, or a combination thereof. In some embodiments, the soluble GFRAL or functional variant thereof comprises the amino acid sequence of SEQ ID NO:3 or a functional variant thereof, or of SEQ ID NO:25 or a functional variant thereof.

In some embodiments, the GDF15 peptide comprises or consists of the amino acid sequence of SEQ ID NO:13 or a functional variant thereof, including amino acid sequences of SEQ ID NO: 14, 15, 16 or 17. In some embodiments, the GDF15 peptide or functional variant has at least 80% amino acid sequence identity with the amino acid sequence of SEQ ID NO:13. In some embodiments, the GDF15 peptide or functional variant has at least 90% amino acid sequence identity with the amino acid sequence of SEQ ID NO:13. In some embodiments, the GDF15 peptide or functional variant has at least 95% amino acid sequence identity with the amino acid sequence of SEQ ID NO:13. In some embodiments, the GDF15 peptide further comprises (e.g., is fused to) an affinity tag, a fusion, a conjugation, a PEGylation, and/or a glycosylation. In some embodiments, the GDF15 peptide is tagged with an amyloid-beta precursor protein tag, a histidine tag, a FLAG tag, or a myc tag, or a combination thereof. In some embodiments, the GDF15 peptide is fused to a human serum albumin, a mouse serum albumin, an immunoglobulin constant region, or an alpha-1-antitrypsin. In some embodiments, the GDF15 peptide is conjugated to a fatty acid.

In certain other aspects, the present disclosure provides a method of producing a pharmaceutical composition comprising an agent, comprising: (a) identifying an agent capable of modulating GDF15 activity by any of the exemplary identification methods described herein; and (b) formulating the agent in a pharmaceutical composition. In some embodiments, the agent is an antibody. In some embodiments, the agent is an anti-GDF15 antibody. In some embodiments, the agent is an anti-GFRAL antibody.

In certain other aspects, the present disclosure provides a method of treating obesity or an obesity-related disorder in a subject, comprising: (a) identifying an agent capable of modulating GDF15 activity by any of the exemplary identification methods described herein; and (b) administering the agent to the subject. In some embodiments, the agent is an antibody. In some embodiments, the agent is an anti-GDF15 antibody. In some embodiments, the agent is an anti-GFRAL antibody. In some embodiments, the subject is overweight or obese. In some embodiments, the subject has a body mass index between 25 and 29.9. In some embodiments, the subject has a body mass index of 30 or higher. In some embodiments, the obesity-related disorder is a cancer, a body weight disorder, or a metabolic disease or disorder. In some embodiments, the obesity-related disorder is a cancer, T2DM, NASH, hypertriglyceridemia, or cardiovascular disease.

In certain other aspects, the present disclosure provides a method of reducing appetite and/or body weight in a subject, comprising: (a) identifying an agent capable of modulating GDF15 activity by any of the exemplary identification methods described herein; and (b) administering the agent to the subject. In some embodiments, the agent is an antibody. In some embodiments, the agent is an anti-GDF15 antibody. In some embodiments, the agent is an anti-GFRAL antibody. In some embodiments, the subject is overweight or obese. In some embodiments, the subject has a body mass index between 25 and 29.9. In some embodiments, the subject has a body mass index of 30 or higher.

In one aspect, provided herein is a method of detecting the activity of a GDF15 peptide, comprising: (i) (a) providing a cell that expresses a cell surface receptor kinase; (b) contacting the cell with the GDF15 peptide and a soluble GFRAL, wherein the soluble GFRAL comprises a GFRAL extracellular domain comprising domains D2 and D3; and (c) detecting a biological response in the contacted cell; or (ii) (a) providing a cell that expresses a cell surface receptor kinase and a GFRAL extracellular domain comprising domains D2 and D3; (b) contacting the cell with the GDF15 peptide; and (c) detecting a biological response in the contacted cell.

In some embodiments, the GFRAL extracellular domain lacks domain D1. In some embodiments, the method comprises providing a cell that expresses a cell surface receptor kinase and a GFRAL extracellular domain, wherein (i) the GFRAL extracellular domain is a soluble GFRAL extracellular domain, or (ii) the GFRAL extracellular domain is attached to the cell surface by a tether.

In some embodiments, the tether: (i) is a GFRAL transmembrane domain or a functional fragment thereof; (ii) comprises the amino acid sequence of SEQ ID NO:18 or a functional variant thereof; (iii) is a heterologous transmembrane domain fused to the GFRAL extracellular domain; (iv) is a glycophosphatidylinositol (GPI); (v) comprises the amino acid sequence of SEQ ID NO:19 or a functional variant thereof, SEQ ID NO:20 or a functional variant thereof, or SEQ ID NO:21 or a functional variant thereof; (vi) is a membrane-inserting sequence, (vii) comprises the amino acid sequence of SEQ ID NO:22 or a functional variant thereof; or SEQ ID NO:23 or a functional variant thereof; or (viii) is a membrane-inserting fatty acid.

In some embodiments, the GFRAL extracellular domain further comprises a signal peptide. In some embodiments, the GFRAL extracellular domain is tagged with an affinity tag selected from an amyloid-beta precursor protein tag, a histidine tag, a FLAG tag, and a myc tag.

In some embodiments, the GFRAL extracellular domain (i) comprises the amino acid sequence of SEQ ID NO:1 or a functional variant thereof; (ii) has at least 80% amino acid sequence identity with the amino acid sequence of SEQ ID NO:1; (iii) has at least 90% amino acid sequence identity with the amino acid sequence of SEQ ID NO:1; (iv) comprises the amino acid sequence of SEQ ID NO:2 or a functional variant thereof; (v) has at least 80% amino acid sequence identity with the amino acid sequence of SEQ ID NO:2; (vi) has at least 90% amino acid sequence identity with the amino acid sequence of SEQ ID NO:2; (vii) comprises the amino acid sequence of SEQ ID NO:3 or a functional variant thereof; or (viii) comprises the amino acid sequence of SEQ ID NO:25 or a functional variant thereof.

In some embodiments, the GDF15 peptide or functional variant thereof (i) comprises the amino acid sequence of SEQ ID NO:13, 14, 15, 16 or 17, or a functional variant thereof; (ii) has at least 80% amino acid sequence identity with the amino acid sequence of SEQ ID NO:13; or (iii) has at least 90% amino acid sequence identity with the amino acid sequence of SEQ ID NO:13.

In some embodiments, the GDF15 peptide (i) is tagged with an affinity tag selected from an amyloid-beta precursor protein tag, a histidine tag, a FLAG tag, and a myc tag; (ii) is fused to a human serum albumin, a mouse serum albumin, an immunoglobulin constant region, or an alpha-1-antitrypsin; (iii) is conjugated to a fatty acid; (iv) has a PEGylation, and/or (v) has a glycosylation. In some embodiments, the cell surface receptor kinase is (i) an endogenous cell surface receptor kinase; (ii) an exogenous cell surface receptor kinase; and/or (iii) a RET receptor tyrosine kinase.

In some embodiments, the cell does not express (i) endogenous GFRAL; (ii) full length GFRAL; and/or (iii) endogenous GDF15. In some embodiments, the cell is a GDF15 knockout (KO) cell comprising an inoperative GDF15 gene.

In some embodiments, the biological response (i) is induced when the GDF15 peptide, the soluble GFRAL or the GFRAL extracellular domain, and the cell surface receptor kinase form a ternary complex; (ii) is not induced in a cell contacted with the GDF15 peptide in the absence of the soluble GFRAL; and/or (iii) is an increase or decrease in the expression or activity of a protein in the cell, as compared to the expression or activity of the same protein in a control cell that is not contacted with the GDF15 peptide and/or the soluble GFRAL. In some embodiments, the biological response is an increase or decrease in the expression or activity of an intracellular protein in one or more of the RET-ERK, RET-AKT, protein kinase C, JAK/STAT, JNK, p38, and RAC1 pathways.

In some embodiments, the cell surface receptor kinase is a RET receptor tyrosine kinase and the protein is (i) an intracellular protein in the RET-ERK pathway selected from ERK1, ERK2, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2, or any downstream targets thereof; or (ii) is an intracellular protein the RET-AKT pathway selected from AKT1, AKT2, AKT3, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR, or any downstream targets thereof. In some embodiments, the biological response is an increase or decrease in phosphorylation of a protein kinase in the cell, as compared to phosphorylation of the same protein kinase in a control cell that is not contacted with the GDF15 peptide and/or the soluble GFRAL.

In some embodiments, (i) the protein kinase is the cell surface receptor kinase; (ii) the protein kinase and/or cell surface receptor kinase is a RET receptor tyrosine kinase; or (iii) the protein kinase is an intracellular protein kinase, wherein the intracellular protein kinase is directly or indirectly phosphorylated by the cell surface receptor kinase. In some embodiments, the protein kinase is (i) an intracellular protein kinase in the RET-ERK pathway selected from of ERK1, ERK2, JAK1, JAK2, RAF, MEK1, MEK2, RSK1, RSK2, RSK3, MNK1, MNK2, MSK1, and MSK2, or any downstream targets thereof; or (ii) an intracellular protein kinase in the RET-AKT pathway selected from AKT1, AKT2, AKT3, SRC, JAK1, JAK2, PI3K, PDK1, MLK3, ASK1, GSK3alpha, GSK3beta, and mTOR, or any downstream targets thereof.

In one aspect, provided herein is an isolated and modified cell for detecting the activity of a GDF15 peptide, wherein the cell expresses a GFRAL extracellular domain comprising domains D2 and D3 and a cell surface receptor kinase.

In some embodiments, the GFRAL extracellular domain lacks domain D1. In some embodiments, the GFRAL extracellular domain is (i) a soluble GFRAL extracellular domain; or (ii) attached to the cell surface by a tether. In some embodiments, the tether (i) is a GFRAL transmembrane domain or a functional fragment thereof; (ii) comprises the amino acid sequence of SEQ ID NO:18 or a functional variant thereof; (iii) is a heterologous transmembrane domain fused to the GFRAL extracellular domain; (iv) is a glycophosphatidylinositol (GPI); (v) comprises the amino acid sequence of SEQ ID NO:19 or a functional variant thereof, SEQ ID NO:20 or a functional variant thereof, or SEQ ID NO:21 or a functional variant thereof; (vi) is a membrane-inserting sequence; (vii) comprises the amino acid sequence of SEQ ID NO:22 or a functional variant thereof, or SEQ ID NO:23 or a functional variant thereof; or (viii) is a membrane-inserting fatty acid.

In some embodiments, the GFRAL extracellular domain further comprises a signal peptide. In some embodiments, the GFRAL extracellular domain is tagged with an affinity tag selected from an amyloid-beta precursor protein tag, a histidine tag, a FLAG tag, and a myc tag.

In some embodiments, the GFRAL extracellular domain (i) comprises the amino acid sequence of SEQ ID NO:1 or a functional variant thereof; (ii) has at least 80% amino acid sequence identity with the amino acid sequence of SEQ ID NO:1; (iii) has at least 90% amino acid sequence identity with the amino acid sequence of SEQ ID NO:1; (iv) comprises the amino acid sequence of SEQ ID NO:2 or a functional variant thereof; (v) has at least 80% amino acid sequence identity with the amino acid sequence of SEQ ID NO:2; (vi) has at least 90% amino acid sequence identity with the amino acid sequence of SEQ ID NO:2; (vii) comprises the amino acid sequence of SEQ ID NO:3 or a functional variant thereof; or (viii) comprises the amino acid sequence of SEQ ID NO:25 or a functional variant thereof. In some embodiments, the cell surface receptor kinase is (i) an endogenous cell surface receptor kinase; (ii) an exogenous cell surface receptor kinase; and/or (iii) a RET receptor tyrosine kinase.

In some embodiments, the cell does not express (i) endogenous GFRAL; (ii) full length GFRAL; and/or (iii) endogenous GDF15. In some embodiments, the cell is a GDF15 knockout (KO) cell comprising an inoperative GDF15 gene. In some embodiments, the cell is selected from a mammalian cell, a human cell, an MCF7 cell, an SH-SY5Y cell, and an HEK293A-GDF15 KO cell.

In one aspect, provided herein is a kit for determining the activity of a GDF15 peptide, wherein the kit comprises the cell of any one of claims 19 to 29 for contacting with the GDF15 peptide; and a means of detecting a biological response in the contacted cell.

In one aspect, provided herein is a soluble GFRAL comprising a GFRAL extracellular domain comprising domains D2 and D3.

In some embodiments, the GFRAL extracellular domain lacks domain D1. In some embodiments, the GFRAL extracellular domain further comprises a signal peptide. In some embodiments, the GFRAL extracellular domain is tagged with an affinity tag selected from an amyloid-beta precursor protein tag, a histidine tag, a FLAG tag, and a myc tag. In some embodiments, the GFRAL extracellular domain: (i) comprises the amino acid sequence of SEQ ID NO:1 or a functional variant thereof; (ii) has at least 80% amino acid sequence identity with the amino acid sequence of SEQ ID NO:1; (iii) has at least 90% amino acid sequence identity with the amino acid sequence of SEQ ID NO:1; (iv) comprises the amino acid sequence of SEQ ID NO:2 or a functional variant thereof; (v) has at least 80% amino acid sequence identity with the amino acid sequence of SEQ ID NO:2; (vi) has at least 90% amino acid sequence identity with the amino acid sequence of SEQ ID NO:2; (vii) comprises the amino acid sequence of SEQ ID NO:3 or a functional variant thereof; or (viii) comprises the amino acid sequence of SEQ ID NO:25 or a functional variant thereof.

In one aspect, provided herein is a method of identifying an agent capable of modulating GDF15 activity, wherein the method comprises: (a) contacting the cell of any one of claims 19 to 29 with the agent and a GDF15 peptide; and (b) detecting a biological response in the contacted cell, wherein the agent is determined to modulate GDF15 activity if the biological response in the contacted cell is increased or decreased relative to the biological response in a cell contacted with the GDF15 peptide in the absence of the agent.

In one aspect, provided herein is a method of identifying an agent capable of modulating GDF15 activity, comprising: (a) providing a cell that expresses a cell surface receptor kinase; (b) contacting the cell with a GDF15 peptide and a soluble GFRAL, wherein the soluble GFRAL comprises a GFRAL extracellular domain comprising domains D2 and D3 and lacks domain D1; (c) contacting the cell with the agent; and (d) detecting a biological response in the contacted cell, wherein the agent is determined to (i) modulate or increase GDF15 activity if the biological response in the contacted cell is increased in the presence of the GDF15 peptide, the soluble GFRAL, and the agent relative to the biological response in a cell contacted with the GDF15 peptide and the soluble GFRAL in the absence of the agent; or (ii) modulate or decrease GDF15 activity if the biological response in the contacted cell is decreased in the presence of the GDF15 peptide, the soluble GFRAL, and the agent relative to the biological response in a cell contacted with the GDF15 peptide and the soluble GFRAL in the absence of the agent.

In some embodiments, the GFRAL extracellular domain is tagged with an affinity tag selected from an amyloid-beta precursor protein tag, a histidine tag, a FLAG tag, and a myc tag. In some embodiments, the GFRAL extracellular domain: (i) comprises the amino acid sequence of SEQ ID NO:1 or a functional variant thereof; (ii) has at least 80% amino acid sequence identity with the amino acid sequence of SEQ ID NO:1; (iii) has at least 90% amino acid sequence identity with the amino acid sequence of SEQ ID NO:1; (iv) comprises the amino acid sequence of SEQ ID NO:2 or a functional variant thereof; (v) has at least 80% amino acid sequence identity with the amino acid sequence of SEQ ID NO:2; (vi) has at least 90% amino acid sequence identity with the amino acid sequence of SEQ ID NO:2; (vii) comprises the amino acid sequence of SEQ ID NO:3 or a functional variant thereof; or (viii) comprises the amino acid sequence of SEQ ID NO:25 or a functional variant thereof. In some embodiments, the agent is an antibody selected from an anti-GDF15 antibody and an anti-GFRAL antibody.

In some embodiments, the biological response is an increase or decrease in the expression, activity, or phosphorylation level of an intracellular protein in one or more of the RET-ERK, RET-AKT, protein kinase C, JAK/STAT, JNK, p38, and RAC1 pathways. In some embodiments, the intracellular protein is in the RET-ERK pathway and is selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2. In some embodiments, the intracellular protein is in the RET-AKT pathway and is selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

In some embodiments, the GDF15 peptide or functional variant thereof: (i) comprises the amino acid sequence of SEQ ID NO:13, 14, 15, 16 or 17, or a functional variant thereof; (ii) has at least 80% amino acid sequence identity with the amino acid sequence of SEQ ID NO:13; or (iii) has at least 90% amino acid sequence identity with the amino acid sequence of SEQ ID NO:13.

In some embodiments, the GDF15 peptide: (i) is tagged with an affinity tag selected from an amyloid-beta precursor protein tag, a histidine tag, a FLAG tag, and a myc tag; (ii) is fused to a human serum albumin, a mouse serum albumin, an immunoglobulin constant region, or an alpha-1-antitrypsin; (iii) is conjugated to a fatty acid; (iv) has a PEGylation; and/or (v) has a glycosylation.

### BRIEF DESCRIPTION OF THE DRAWINGS

FIG. 1 shows exemplary human GFRAL extracellular domain (ECD) constructs. Full length GFRAL(ECD)-His was created by deletion of the transmembrane domain and the C-terminal cytoplasmic tail, and addition of a six-histidine (His) tag. GFRAL(D2D3)-App was created by deletion of domain D1 (GDNF receptor (GFRα1) homologue D1) and the membrane proximal region (X), and addition of an amyloid beta precursor protein (App) epitope tag. GFRAL(D2D3)-His was created by deletion of domain D1 (GDNF receptor (GFRα1) homologue D1) and the membrane proximal region (X), and addition of a six-histidine (His) tag. GFRAL(ECD)-Fc was created by fusing the GFRAL(ECD) to the human immunoglobulin G1 (IgG1) constant region (Fc) domain. Human cRET(ECD)-Fc was created by fusion of human RET extracellular domain (RET-ECD) with human IgG1 Fc. His-GDF15, an N-terminally six histidine-tagged human GDF15, was also created. Abbreviations: S.P.—CD33 signal peptide; D1-D3—domains D1-D3 of GDNF receptor (GFRα1) homologue; App—amyloid beta precursor protein; X—region of unknown function between domain D3 and transmembrane domain; ECD—extracellular domain; RET—rearranged during transfection.

FIG. 2A shows exemplary His-GDF15 and GFRAL(D2D3)-App constructs. FIG. 2B shows screening of fractions by SDS-PAGE analysis. Single protein bands shown in FIG. 2B contain both GFRAL(D2D3)-App monomer (24.6 kD) and His-GDF15 dimer (26.6 kD for dimer, 13.3 kD for monomer).

FIG. 3 shows that the complex concentrated from several fractions contains co-expressed GFRAL(D2D3)-App and His-GDF15, as revealed by SDS-PAGE under reducing conditions. The complex contains 24.6 kD GFRAL(D2D3)-App and 13.3 kD His-GDF15.

FIG. 4A shows fractions that contain His-GDF15/GFRAL(ECD)-Fc complex, analyzed by SDS-PAGE under non-reducing conditions. FIG. 4B shows that the complex concentrated from fractions in FIG. 4A contains His-GDF15 and GFRAL(ECD)-Fc, as revealed by SDS-PAGE under reducing conditions.

FIG. 5 shows binding activity of purified His-GDF15 complexes combined with purified recombinant soluble GFRAL ECD variants to cRET(ECD)-Fc coated plates at varying protein concentrations (0-5 log 10 pM).

FIG. 6 shows binding activity of purified mixtures of His-GDF15 and GFRAL(ECD)-His or His-GDF15(L294R) and GFRAL(ECD)-His to cRET(ECD)-Fc coated plates at varying protein concentrations (0-5 log 10 pM).

FIG. 7 shows phosphorylation of ERK and AKT in SH-SY5Y cells following 15 min treatment with media (lane 1); GDNF—3.3 nM (+control for GFRα/RET) (lane 2); purified His-GDF15/GFRAL(D2D3)-App complex—27.8 nM (lane 3); purified His-GDF15/GFRAL(D2D3)-App complex—83.3 nM (lane 4); purified His-GDF15/GFRAL(D2D3)-App complex—250 nM (lane 5); purified His-GDF15/GFRAL(ECD)-Fc complex—27.8 nM (lane 6); purified His-GDF15/GFRAL(ECD)-Fc complex—83.3 nM (lane 7); purified His-GDF15/GFRAL(ECD)-Fc complex—250 nM (lane 8); His-GDF15 alone—250 nM (lane 9); GFRAL(D2D3)-App alone—250 nM (lane 10); GFRAL(ECD)-Fc alone—250 nM (lane 11); His-GDF15+GFRAL(D2D3)-App formed in medium for 60 min prior to addition to cells—250 nM each component (lane 12), as analyzed by Western blot. For lanes 3-8, GDF15/GFRAL complexes were co-expressed and co-purified from supernatants of co-transfected HEK293F cells.

FIG. 8 shows phosphorylation of ERK and AKT in MCF7 cells following 15 min treatment with media (lane 1); GDNF—3.3 nM (+ control for GFRα/RET) (lane 2); purified His-GDF15/GFRAL(D2D3)-App complex—27.8 nM (lane 3); purified His-GDF15/GFRAL(D2D3)-App complex—83.3 nM (lane 4); purified His-GDF15/GFRAL(D2D3)-App complex—250 nM (lane 5); purified His-GDF15/GFRAL(ECD)-Fc complex—27.8 nM (lane 6); purified His-GDF15/GFRAL(ECD)-Fc complex—83.3 nM (lane 7); purified His-GDF15/GFRAL(ECD)-Fc complex—250 nM (lane 8); His-GDF15 alone—250 nM (lane 9); GFRAL(D2D3)-App alone—250 nM (lane 10); GFRAL(ECD)-Fc alone—250 nM (lane 11); and His-GDF15+GFRAL(D2D3)-App formed in medium for 60 min prior to addition to cells—250 nM each component (lane 12), as analyzed by Western blot. For lanes 3-8, GDF15/GFRAL complexes were co-expressed and co-purified from supernatants of co-transfected HEK293F cells.

FIG. 9 shows concentration-dependent phosphorylation of ERK and AKT in SH-SY5Y and MCF7 cells following 15 min treatment with media (lane 1); GDNF—3.3 nM (lane 2); GFRAL(D2D3)-App+His-GDF15—28 nM (each) (lane 3); GFRAL(D2D3)-App+His-GDF15—83 nM (lane 4); GFRAL(D2D3)-App+His-GDF15—250 nM (lane 5); media (lane 7); GDNF—3.3 nM (lane 8); GFRAL(D2D3)-App+His-GDF15—28 nM (lane 9); GFRAL(D2D3)-App+His-GDF15—83 nM (lane 10); and GFRAL(D2D3)-App+His-GDF15—250 nM (lane 12), as analyzed by Western blot. GFRAL(D2D3)-App+His-GDF15 was mixed in media and incubated for 1 hour at room temperature prior to treatment.

FIGS. 10A-B show phosphorylation of ERK and AKT in MCF7 cells (FIG. 10A) and SH-SY5Y cells (FIG. 10B) following 5-15 min treatment with GDNF—15 min (lane 1); media—5 min (lane 2); GFRAL(D2D3)-App+His-GDF15—5 min (lane 3); media—10 min (lane 4); GFRAL(D2D3)-App+His-GDF15—10 min (lane 5); media—15 min (lane 6); GFRAL(D2D3)-App+His-GDF15—15 min (lane 7); media—15 min (lane 8); and GFRAL(D2D3)-App+His-GDF15—15 min, no pre-incubation of complex (lane 9), as analyzed by Western blot.

FIGS. 11A-B show potency of different forms of purified GDF15 protein on MCF7 cell ERK phosphorylation induction when reconstituted (pre-mixed) with GFRAL(D2D3)-App or full length GFRAL(ECD). Data are expressed as absolute phospho-ERK AlphaLISA assay signal units (FIG. 11A) and fold increase in phosphorylated ERK signal over media control (FIG. 11B).

FIGS. 12A-B show potency of different forms of purified GDF15 protein on SH-SY5Y cell ERK phosphorylation induction when reconstituted (pre-mixed) with GFRAL(D2D3)-App or full length GFRAL(ECD). Data are expressed as absolute phospho-ERK AlphaLISA assay signal units (FIG. 12A) and fold increase in phosphorylated ERK signal over media control (FIG. 12B).

### DETAILED DESCRIPTION OF ILLUSTRATIVE EMBODIMENTS

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

As used herein, “GFRAL” or “GDNF family receptor alpha-like” refers to a GFRAL receptor polypeptide having the amino acid sequence of any naturally-occurring full length GFRAL receptor polypeptide, or any variant or functional fragment thereof which is capable of (1) binding to GDF15; and (2) promoting a biological response associated with a full length GFRAL, such as binding to and activating a RET cell surface receptor when in complex with GDF15 and/or promoting intracellular signaling (e.g., RET-ERK signaling, RET-AKT signaling) in response to GDF15 stimulation. In some embodiments, the GFRAL is a full length mammalian GFRAL (e.g., human, monkey, rat, or mouse GFRAL), or a variant or functional fragment thereof. In some embodiments, the GFRAL is full length human GFRAL, or a variant or functional fragment thereof. Exemplary amino acid and nucleic acid sequences for human GFRAL are provided herein. See, e.g., Table 1, which includes exemplary sequences for full length human GFRAL (amino acid: SEQ ID NO:9 (UniProt Ref. Sequence: Q6UXV0); nucleic acid: SEQ ID NO: 24 (NCBI Ref. Sequence: NM_207410.2)), as well as exemplary variants and functional fragments thereof. Exemplary human GFRAL is also described in Li et al. (J Neurochem 2005; 95(2):361-76) and WO 2003/076569.

The term “receptor,” as used herein, refers to a cell-associated protein that binds to a bioactive molecule termed a “ligand.” In some embodiments, the receptor is GFRAL and the ligand is GDF15. The GFRAL receptor polypeptides of the present disclosure can be “membrane-bound” or “soluble.”

As used herein, “GFRAL ligand” refers to a bioactive molecule that binds to a GFRAL receptor polypeptide, or a variant or functional fragment thereof. A GFRAL ligand can be an antagonist or an agonist of GFRAL. In some embodiments, the GFRAL ligand is a GDF15 peptide. In some embodiments, the GDF15 peptide can be a full length peptide or a fragment that retains the ability to agonize or antagonize GFRAL. In some embodiments, a peptide or fragment can be further conjugated or fused to additional peptides or other therapeutic, pharmacokinetic, or carrier moieties. In some embodiments, the GDF15 peptide can be conjugated to a fatty acid. For example, the fatty acid-conjugated GDF15 peptide can be any such peptide disclosed in WO 2015/200078, which is incorporated herein by reference. In some other embodiments, the GDF15 peptide can be fused to a serum albumin, e.g., human serum albumin (HSA) or mouse serum albumin (MSA). In still other embodiments, the GDF15 peptide can be fused to alpha-1-antitrypsin or to an immunoglobulin constant region (e.g., an immunoglobulin G1 constant region). The GDF15 fusion peptide, for example, can be any such peptide disclosed in WO 2015/198199 or WO 2017/109706, which are both incorporated herein by reference. In some other embodiments, a peptide or fragment can be further modified, e.g., by incorporating sequence variations, by the addition of short peptide sequences to the N- and/or C-terminus of the peptide or fragment, and/or by PEGylation and/or glycosylation, such that the modified peptide or fragment retains one or more functions of an unmodified GDF15 peptide.

As used herein, “soluble GFRAL” refers to a GFRAL receptor polypeptide, or a variant or functional fragment thereof, that is not bound to or anchored into a cell membrane. Soluble receptor polypeptides are commonly ligand-binding receptor polypeptides that lack transmembrane and intracellular domains, or other linkages to the cell membrane, such as glycophosphoinositol. Soluble receptor polypeptides can comprise additional amino acid residues, such as immunoglobulin constant region sequences (e.g., a human immunoglobulin G1 Fc sequence), or affinity tags that provide for purification of the polypeptide or provide sites for attachment of the polypeptide to a substrate (e.g., an amyloid-beta precursor protein (App) tag or a histidine (His) tag). Soluble receptor polypeptides are generally substantially free of transmembrane and intracellular polypeptide segments when they lack sufficient portions of these segments to provide membrane anchoring or signal transduction, respectively. In some embodiments, the GFRAL is a soluble GFRAL (e.g., a soluble human GFRAL). In some embodiments, the soluble GFRAL comprises a GFRAL extracellular domain. In some embodiments, the soluble GFRAL comprises a GFRAL extracellular domain that lacks a sufficient length of a transmembrane domain, such that it is not present on or anchored into a cell membrane.

As used herein, “membrane-bound GFRAL” refers to a GFRAL receptor polypeptide, or a variant or functional fragment thereof, that is bound to or anchored into a cell membrane. In some embodiments, the GFRAL is a membrane-bound GFRAL (e.g., a membrane-bound human GFRAL). In some embodiments, the membrane-bound GFRAL comprises a GFRAL extracellular domain. In some embodiments, the membrane-bound GFRAL comprises a GFRAL extracellular domain that is tethered to the cell surface.

The term “tethered,” as used herein, refers a physical modification of a polypeptide (e.g., the addition of a domain or fatty acylation site that causes the polypeptide to be localized to the cell surface). In some embodiments, the GFRAL extracellular domain is attached to the cell surface by a tether. In some embodiments, the tether is a GFRAL transmembrane domain. In some embodiments, the tether is a GFRAL transmembrane domain functional fragment. In some embodiments, the GFRAL extracellular domain or tether comprises the amino acid sequence of a native GFRAL transmembrane domain. In some embodiments, the GFRAL extracellular domain or tether comprises the amino acid sequence of SEQ ID NO:18 or a functional variant thereof. In some embodiments, the tether is a heterologous transmembrane domain fused to the GFRAL extracellular domain.

In some embodiments, the tether is a glycophosphatidylinositol (GPI) or a sequence capable of directing GPI linker addition. In some embodiments, the GFRAL extracellular domain or tether comprises the amino acid sequence of SEQ ID NO:19 or a functional variant thereof, SEQ ID NO:20 or a functional variant thereof, or SEQ ID NO:21 or a functional variant thereof. In some embodiments, the tether is a membrane-inserting sequence, e.g., an autonomously membrane-inserting sequence or a sequence described in Vergeres et al., J Biol Chem 1995; 270(7):3414-22, which is incorporated herein by reference. Exemplary membrane-inserting sequences include but are not limited to the membrane-spanning C-terminal domain of cytochrome b5 (SEQ ID NOs:22 and 23) (see, e.g., Vergeres et al., J Biol Chem 1995; 270(7):3414-22). In some embodiments, the GFRAL extracellular domain or tether comprises the amino acid sequence of SEQ ID NO:22 or a functional variant thereof, or SEQ ID NO:23 or a functional variant thereof. In some other embodiments, the tether is a membrane-inserting fatty acid. In some other embodiments, the tether is a heterologous transmembrane domain which is fused to an extracellular domain of GFRAL. In some embodiments, the transmembrane domain localizes the GFRAL extracellular domain to the cell surface.

As used herein, the term “variant” refers to a sequence that has been modified with respect to a reference (unmodified) native amino acid sequence. A modified sequence contains one or more amino acid substitutions, deletions, and/or insertions (or corresponding substitution, deletion, and/or insertion of codons) with respect to a reference sequence. A variant does not necessarily require physical manipulation of the reference sequence. As long as a sequence contains a different amino acid as compared to a reference sequence, it is considered a “variant” regardless of how it was synthesized. In certain embodiments, a variant has high amino acid sequence homology as compared to a reference sequence. In some embodiments, a variant encompasses polypeptides having amino acid substitutions, deletions, and/or insertions as long as the polypeptide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% amino acid sequence identity with a reference sequence, or with a corresponding segment (e.g., a functional fragment) of a reference sequence. The reference sequence may be, for example, human GFRAL (SEQ ID NO:9; UniProt Ref. Sequence: Q6UXV0). The reference sequence may also be, for example, a functional fragment of human GFRAL, such as the full length extracellular binding domain of human GFRAL (SEQ ID NO:4; amino acids 20-351 of UniProt Ref. Sequence: Q6UXV0). In some embodiments, the GFRAL extracellular domain is a variant of the full length extracellular binding domain of human GFRAL.

In some embodiments, the GFRAL extracellular domain comprises domains D2 and D3, but lacks domain D1 (see, e.g., SEQ ID NO:1). In some embodiments, the reference sequence is SEQ ID NO:1. In some embodiments, the GFRAL and/or GFRAL extracellular domain has at least 80% amino acid sequence identity with the amino acid sequence of SEQ ID NO:1. In some embodiments, the GFRAL and/or GFRAL extracellular domain has at least 85% amino acid sequence identity with the amino acid sequence of SEQ ID NO:1. In some embodiments, the GFRAL and/or GFRAL extracellular domain has at least 90% amino acid sequence identity with the amino acid sequence of SEQ ID NO:1. In some embodiments, the GFRAL and/or GFRAL extracellular domain has at least 95%, 96%, 97%, 98%, or 99% amino acid sequence identity with the amino acid sequence of SEQ ID NO:1.

In some embodiments, the GFRAL extracellular domain comprising domains D2 and D3 but lacking domain D1 further comprises a signal peptide (see, e.g., SEQ ID NO:2). In some embodiments, the reference sequence is SEQ ID NO:2. In some embodiments, the GFRAL and/or GFRAL extracellular domain has at least 80% amino acid sequence identity with the amino acid sequence of SEQ ID NO:2. In some embodiments, the GFRAL and/or GFRAL extracellular domain has at least 85% amino acid sequence identity with the amino acid sequence of SEQ ID NO:2. In some embodiments, the GFRAL and/or GFRAL extracellular domain has at least 90% amino acid sequence identity with the amino acid sequence of SEQ ID NO:2. In some embodiments, the GFRAL and/or GFRAL extracellular domain has at least 95%, 96%, 97%, 98%, or 99% amino acid sequence identity with the amino acid sequence of SEQ ID NO:2.

In some embodiments, the GFRAL extracellular domain comprising domains D2 and D3 but lacking domain D1 further comprises (e.g., is fused to) an affinity tag (see, e.g., SEQ ID NO:3 or SEQ ID NO:25). In some embodiments, the reference sequence is SEQ ID NO:3. In some embodiments, the reference sequence is SEQ ID NO:25. In some embodiments, the GFRAL and/or GFRAL extracellular domain has at least 80% amino acid sequence identity with the amino acid sequence of SEQ ID NO:3 or SEQ ID NO:25. In some embodiments, the GFRAL and/or GFRAL extracellular domain has at least 85% amino acid sequence identity with the amino acid sequence of SEQ ID NO:3 or SEQ ID NO:25. In some embodiments, the GFRAL and/or GFRAL extracellular domain has at least 90% amino acid sequence identity with the amino acid sequence of SEQ ID NO:3 or SEQ ID NO:25. In some embodiments, the GFRAL and/or GFRAL extracellular domain has at least 95%, 96%, 97%, 98%, or 99% amino acid sequence identity with the amino acid sequence of SEQ ID NO:3 or SEQ ID NO:25.

The term “identity” or “homology” refers to a relationship between the sequences of two or more polypeptides, as determined by comparing the sequences. “Identity” also means the degree of sequence relatedness between polypeptides, as determined by the number of matches between strings of two or more amino acid residues. “Identity” measures the percent of identical matches between the smaller of two or more sequences with gap alignments (if any) addressed by a mathematical model or computer program (i.e., algorithms). Identity of related proteins is capable of being readily calculated by known methods. Such methods include, but are not limited to, those described in “Computational Molecular Biology” (Lesk A M, ed., Oxford University Press, New York, 1988); and “Biocomputing: Informatics and Genome Projects” (Smith D W, ed., Academic Press, New York, 1993).

In some embodiments, the GFRAL used in the compositions and methods described herein is a variant of GFRAL, e.g., a variant of human GFRAL, or a functional fragment thereof. Such variants are encompassed by the term “GFRAL.” The term “GFRAL variant” refers to a GFRAL variant that retains the ability to (1) bind to GDF15; and (2) promote a biological response associated with a full length GFRAL, such as binding to and activating a RET cell surface receptor when in complex with GDF15 and/or promoting intracellular signaling (e.g., RET-ERK signaling, RET-AKT signaling). A GFRAL variant can be a truncated GFRAL, a GFRAL analogue, or a GFRAL derivative. The term “truncated GFRAL” means a functional fragment of wild-type GFRAL. A functional fragment of wild-type GFRAL may comprise, for example, a full length GFRAL extracellular domain or a GFRAL extracellular domain comprising only domains D2 and D3. The term “GFRAL analogue” means a modified GFRAL, wherein one or more amino acid residues of wild-type GFRAL have been substituted with other natural or unnatural amino acid residues, and/or wherein one or more natural or unnatural amino acid residues have been added to wild-type GFRAL. The term “GFRAL derivative” means a chemically-modified wild-type GFRAL with or without substituting, adding, or deleting one or more natural or unnatural amino acid residues, wherein at least one substituent is not present in wild-type GFRAL. Typical modifications include but are not limited to amides, carbohydrates, alkyl groups, acyl groups, esters, and PEGylations.

As used herein, “GFRAL extracellular domain” refers to a GFRAL receptor polypeptide that lacks the transmembrane and intracellular (cytoplasmic) domains. A GFRAL extracellular domain may or may not include an N-terminal signal peptide and may be derived from any species. In some embodiments, the GFRAL extracellular domain is the extracellular domain of a mammalian GFRAL (e.g., human, monkey, rat, or mouse GFRAL). In some embodiments, the GFRAL extracellular domain is the extracellular domain of human GFRAL. The term “GFRAL extracellular domain” includes wild-type GFRAL extracellular domains and GFRAL extracellular domain variants.

Within the GFRAL extracellular domain, there are three separate cysteine-rich subdomains: domain 1 (D1), domain 2 (D2), and domain 3 (D3). Certain properties of GFRAL can be attributed to the activity and/or binding affinity of these subdomains. For example, amino acid residues within domain D2 have been identified as being interaction interface amino acids for GFRAL binding to GDF15. Likewise, amino acid residues within domain D3 have been identified as being interaction interface amino acids for GFRAL binding to RET. See, e.g., WO 2017/152105. The term “GFRAL extracellular domain” encompasses GFRAL extracellular domains comprising one, two, or all three of these domains (D1, D2, and D3). In some embodiments, the GFRAL extracellular domain comprises domains D1, D2, and D3. In other embodiments, the GFRAL extracellular domain comprises domains D2 and D3, but lacks domain D1. In some embodiments, the GFRAL extracellular domain comprising domains D2 and D3 but lacking domain D1 further comprises an N-terminal signal peptide.

Exemplary GFRAL sequences and constructs are set forth in Table 1.

TABLE 1
Exemplary GFRAL sequences and constructs.

| | SEQ | |
|---|---|---|
| | ID | |
| Name | NO | Amino acid sequence* |
| Human | 1 | GFKGMWSCLEVAEACVGDVVCNAQLASYLKACSANGNPCDLKQC |
| GFRAL(D2D3) | | QAAIRFFYQNIPFNIAQMLAFCDCAQSDIPCQQSKEALHSKTCA |
| (without signal | | VNMVPPPTCLSVIRSCQNDELCRRHYRTFQSKCWQRVTRKCHED |
| peptide) | | ENCISTLSKQDLTCSGSDDCKAAYIDILGTVLQVQCTCRTITQS |
| | | EESLCKIFQHMLHRKSCFNYPTLSNVKGMALYTRK |
| Human | 2 | MPLLLLLPLLWAGALAGFKGMWSCLEVAEACVGDVVCNAQLASY |
| GFRAL(D2D3) | | LKACSANGNPCDLKQCQAAIRFFYQNIPFNIAQMLAFCDCAQSD |
| | | IPCQQSKEALHSKTCAVNMVPPPTCLSVIRSCQNDELCRRHYRT |
| | | FQSKCWQRVTRKCHEDENCISTLSKQDLTCSGSDDCKAAYIDIL |
| | | GTVLQVQCTCRTITQSEESLCKIFQHMLHRKSCFNYPTLSNVKG |
| | | MALYTRK |
| Human | 3 | MPLLLLLPLLWAGALAGFKGMWSCLEVAEACVGDVVCNAQLASY |
| GFRAL(D2D3)- | | LKACSANGNPCDLKQCQAAIRFFYQNIPFNIAQMLAFCDCAQSD |
| App | | IPCQQSKEALHSKTCAVNMVPPPTCLSVIRSCQNDELCRRHYRT |
| | | FQSKCWQRVTRKCHEDENCISTLSKQDLTCSGSDDCKAAYIDIL |
| | | GTVLQVQCTCRTITQSEESLCKIFQHMLHRKSCFNYPTLSNVKG |
| | | MALYTRKGSEFRHDS |
| Human | 25 | MPLLLLLPLLWAGALAGFKGMWSCLEVAEACVGDVVCNAQLASY |
| GFRAL(D2D3)- | | LKACSANGNPCDLKQCQAAIRFFYQNIPFNIAQMLAFCDCAQSD |
| His | | IPCQQSKEALHSKTCAVNMVPPPTCLSVIRSCQNDELCRRHYRT |
| | | FQSKCWQRVTRKCHEDENCISTLSKQDLTCSGSDDCKAAYIDIL |
| | | GTVLQVQCTCRTITQSEESLCKIFQHMLHRKSCFNYPTLSNVKG |
| | | MALYTRKGSHHHHHH |
| Human | 4 | QTNNCTYLREQCLRDANGCKHAWRVMEDACNDSDPGDPCKMRNS |
| GFRAL(ECD) | | SYCNLSIQYLVESNFQFKECLCTDDFYCTVNKLLGKKCINKSDN |
| (without signal | | VKEDKEKWNLTTRSHHGEKGMWSCLEVAEACVGDVVCNAQLASY |
| peptide) | | LKACSANGNPCDLKQCQAAIRFFYQNIPFNIAQMLAFCDCAQSD |
| | | IPCQQSKEALHSKTCAVNMVPPPTCLSVIRSCQNDELCRRHYRT |
| | | FQSKCWQRVTRKCHEDENCISTLSKQDLTCSGSDDCKAAYIDIL |
| | | GTVLQVQCTCRTITQSEESLCKIFQHMLHRKSCFNYPTLSNVKG |
| | | MALYTRKHANKITLTGEHSPENGE |
| Human | 5 | MPLLLLLPLLWAGALAQTNNCTYLREQCLRDANGCKHAWRVMED |
| GFRAL(ECD) | | ACNDSDPGDPCKMRNSSYCNLSIQYLVESNFQFKECLCTDDFYC |
| | | TVNKLLGKKCINKSDNVKEDKFKWNLTTRSHHGFKGMWSCLEVA |
| | | EACVGDVVCNAQLASYLKACSANGNPCDLKQCQAAIRFFYQNIP |
| | | FNIAQMLAFCDCAQSDIPCQQSKEALHSKTCAVNMVPPPTCLSV |
| | | IRSCQNDELCRRHYRTFQSKCWQRVTRKCHEDENCISTLSKQDL |
| | | TCSGSDDCKAAYIDILGTVLQVQCTCRTITQSEESLCKIFQHML |
| | | HRKSCFNYPTLSNVKGMALYTRKHANKITLTGFHSPFNGE |
| Human | 6 | MPLLLLLPLLWAGALAQTNNCTYLREQCLRDANGCKHAWRVMED |
| GFRAL(ECD)-His | | ACNDSDPGDPCKMRNSSYCNLSIQYLVESNFQFKECLCTDDFYC |
| | | TVNKLLGKKCINKSDNVKEDKFKWNLTTRSHHGFKGMWSCLEVA |
| | | EACVGDVVCNAQLASYLKACSANGNPCDLKQCQAAIRFFYQNIP |
| | | FNIAQMLAFCDCAQSDIPCQQSKEALHSKTCAVNMVPPPTCLSV |
| | | IRSCQNDELCRRHYRTFQSKCWQRVTRKCHEDENCISTLSKQDL |
| | | TCSGSDDCKAAYIDILGTVLQVQCTCRTITQSEESLCKIFQHML |
| | | HRKSCFNYPTLSNVKGMALYTRKHANKITLTGFHSPFNGEGSHH |
| | | HHHH |
| Human | 7 | MPLLLLLPLLWAGALAQTNNCTYLREQCLRDANGCKHAWRVMED |
| GFRAL(ECD)-Fc | | ACNDSDPGDPCKMRNSSYCNLSIQYLVESNFQFKECLCTDDFYC |
| | | TVNKLLGKKCINKSDNVKEDKFKWNLTTRSHHGFKGMWSCLEVA |
| | | EACVGDVVCNAQLASYLKACSANGNPCDLKQCQAAIRFFYQNIP |
| | | FNIAQMLAFCDCAQSDIPCQQSKEALHSKTCAVNMVPPPTCLSV |
| | | IRSCQNDELCRRHYRTFQSKCWQRVTRKCHEDENCISTLSKQDL |
| | | TCSGSDDCKAAYIDILGTVLQVQCTCRTITQSEESLCKIFQHML |
| | | HRKSCFNYPTLSNVKGMALYTRKHANKITLTGFHSPFNGEGSRI |
| | | PKVDKKVEPKSCDKTHTCPPCPAPELLGGPSVFLFPPKPKDTLM |
| | | ISRTPEVTCVVVDVSHEDPEVKFNWYVDGVEVHNAKTKPREEQY |
| | | NSTYRVVSVLTVLHQDWLNGKEYKCRVSNKALPAPIEKTISKAK |
| | | GQPREPQVYTLPPSRDELTKNQVSLTCLVKGFYPSDIAVEWESN |
| | | GQPENNYKTTPPVLDSDGSFFLYSKLTVDKSRWQQGNVFSCSVM |
| | | HEALHNHYTQKSLSLSPGK |
| Human | 8 | MPLLLLLPLLWAGALALYFSRDAYWEKLYVDQAAGTPLLYVHAL |
| cRET(ECD)-Fc | | RDAPEEVPSFRLGQHLYGTYRTRLHENNWICIQEDTGLLYLNRS |
| | | LDHSSWEKLSVRNRGFPLLTVYLKVFLSPTSLREGECQWPGCAR |
| | | VYFSFENTSFPACSSLKPRELCFPETRPSFRIRENRPPGTFHQF |
| | | RLLPVQFLCPNISVAYRLLEGEGLPFRCAPDSLEVSTRWALDRE |
| | | QREKYELVAVCTVHAGAREEVVMVPFPVTVYDEDDSAPTFPAGV |
| | | DTASAVVEFKRKEDTVVATLRVFDADVVPASGELVRRYTSTLLP |
| | | GDTWAQQTFRVEHWPNETSVQANGSFVRATVHDYRLVLNRNLSI |
| | | SENRTMQLAVLVNDSDFQGPGAGVLLLHFNVSVLPVSLHLPSTY |
| | | SLSVSRRARRFAQIGKVCVENCQAFSGINVQYKLHSSGANCSTL |
| | | GVVTSAEDTSGILFVNDTKALRRPKCAELHYMVVATDQQTSRQA |
| | | QAQLLVTVEGSYVAEEAGCPLSCAVSKRRLECEECGGLGSPTGR |
| | | CEWRQGDGKGITRNFSTCSPSTKTCPDGHCDVVETQDINICPQD |
| | | CLRGSIVGGHEPGEPRGIKAGYGTCNCFPEEEKCFCEPEDIQDP |
| | | LCDELCRGSRIPKVDKKVEPKSCDKTHTCPPCPAPELLGGPSVF |
| | | LFPPKPKDTLMISRTPEVTCVVVDVSHEDPEVKFNWYVDGVEVH |
| | | NAKTKPREEQYNSTYRVVSVLTVLHQDWLNGKEYKCRVSNKALP |
| | | APIEKTISKAKGQPREPQVYTLPPSRDELTKNQVSLTCLVKGFY |
| | | PSDIAVEWESNGQPENNYKTTPPVLDSDGSFFLYSKLTVDKSRW |
| | | QQGNVFSCSVMHEALHNHYTQKSLSLSPGK |
| Human GFRAL | 9 | MIVFIFLAMGLSLENEYTSQTNNCTYLREQCLRDANGCKHAWRV |
| (full length amino | | MEDACNDSDPGDPCKMRNSSYCNLSIQYLVESNFQFKECLCTDD |
| acid sequence) | | FYCTVNKLLGKKCINKSDNVKEDKEKWNLTTRSHHGEKGMWSCL |
| | | EVAEACVGDVVCNAQLASYLKACSANGNPCDLKQCQAAIRFFYQ |
| | | NIPFNIAQMLAFCDCAQSDIPCQQSKEALHSKTCAVNMVPPPTC |
| | | LSVIRSCQNDELCRRHYRTFQSKCWQRVTRKCHEDENCISTLSK |
| | | QDLTCSGSDDCKAAYIDILGTVLQVQCTCRTITQSEESLCKIFQ |
| | | HMLHRKSCFNYPTLSNVKGMALYTRKHANKITLTGEHSPENGEV |
| | | IYAAMCMTVTCGILLLVMVKLRTSRISSKARDPSSIQIPGEL |
| CD33 signal | 10 | MPLLLLLPLLWAGALA |
| peptide | | |
| Human GFRAL | 18 | VIYAAMCMTVTCGILLLVMV |
| (transmembrane | | |
| domain) | | |
| Exemplary tether | 19 | GSGTTSGTTRLLSSHTSAALTVLSVLMLKLAL |
| sequence (GPI) | | |
| Exemplary tether | 20 | SGPSRARPSAALTVLSVLMLKLAL |
| sequence (GPI) | | |
| Exemplary tether | 21 | GSGTTSGTTRLLSGHTCFTLTGLLGTLVTMGLLT |
| sequence (GPI) | | |
| Exemplary tether | 22 | DSSSSWWTNWVIPAISAVAVALMYRLYMAED |
| sequence | | |
| (cytochrome b5) | | |
| Exemplary tether | 23 | ESDSSWWTNWVIPAISALVVALMYRLYMAED |
| sequence | | |
| (cytochrome b5) | | |
| Human GFRAL | 24 | TTATTCTGGACAGTTACTCTTAAGAAAGTTGTCAGAAGAAACGC |
| (full length | | ATCTGCCTTTTTTTCCAGGTGAACTGCCGTGAGTTGTCCAGCAT |
| nucleic | | GATAGTGTTTATTTTCTTGGCTATGGGGTTAAGCTTGGAAAATG |
| acid sequence)** | | AATACACTTCCCAAACCAATAATTGCACATATTTAAGAGAGCAA |
| | | TGCTTACGTGATGCAAATGGATGTAAACATGCTTGGAGAGTAAT |
| | | GGAAGATGCCTGCAATGATTCAGATCCAGGTGACCCCTGCAAGA |
| | | TGAGGAATTCATCATACTGTAACCTGAGTATCCAGTACTTAGTG |
| | | GAAAGCAATTTCCAATTTAAAGAGTGTCTTTGCACTGATGACTT |
| | | CTATTGTACTGTGAACAAACTGCTTGGAAAAAAATGTATCAATA |
| | | AATCAGATAACGTGAAAGAGGATAAATTCAAATGGAATCTAACT |
| | | ACACGTTCCCATCATGGATTCAAAGGGATGTGGTCCTGTTTGGA |
| | | AGTGGCAGAGGCATGTGTAGGGGATGTGGTCTGTAATGCACAGT |
| | | TGGCCTCTTACCTTAAAGCTTGCTCAGCAAATGGAAATCCGTGT |
| | | GATCTGAAACAGTGCCAAGCAGCCATACGGTTCTTCTATCAAAA |
| | | TATACCTTTTAACATTGCCCAGATGTTGGCTTTTTGTGACTGTG |
| | | CTCAATCTGATATACCTTGTCAGCAGTCCAAAGAAGCTCTTCAC |
| | | AGCAAGACATGTGCAGTGAACATGGTTCCACCCCCTACTTGCCT |
| | | CAGTGTAATTCGCAGCTGCCAAAATGATGAATTATGCAGGAGGC |
| | | ACTATAGAACATTTCAGTCAAAATGCTGGCAGCGTGTGACTAGA |
| | | AAGTGCCATGAAGATGAGAATTGCATTAGCACCTTAAGCAAACA |
| | | GGACCTCACTTGTTCAGGAAGTGATGACTGCAAAGCTGCTTACA |
| | | TAGATATCCTTGGGACGGTCCTTCAAGTGCAATGTACCTGTAGG |
| | | ACCATTACACAAAGTGAGGAATCTTTGTGTAAGATTTTCCAGCA |
| | | CATGCTTCATAGAAAATCATGTTTCAATTATCCAACCCTGTCTA |
| | | ATGTCAAAGGCATGGCATTGTATACAAGAAAACATGCAAACAAA |
| | | ATCACTTTAACTGGATTTCATTCCCCCTTCAATGGAGAAGTAAT |
| | | CTATGCTGCCATGTGCATGACAGTCACCTGTGGAATCCTTCTGT |
| | | TGGTTATGGTCAAGCTTAGAACTTCCAGAATATCAAGTAAAGCA |
| | | AGAGATCCTTCATCGATCCAAATACCTGGAGAACTCTGATTCAT |
| | | TAGGAGTCATGGACCTATAACAATCACTCTTTTCTCTGCTTTTC |
| | | TTCTTTCCTCTTTTCTTCTCTCCTCTCCTCTCCTCTCTTCTCCT |
| | | CTCCTCCCCTCCCCTCTCTGTTTCTTTTTCTTTTTCTTTTCTTT |
| | | TTTGTGGCGGAGTTTTGCTCTTGTTGCCCAGGCTGCAGTACAAT |
| | | GGCTCAATCTCGGTTCACTGCAACCTCTGCCTCCAAGGTTCAAG |
| | | TGATTTTCCTGCCTCAGCCTTCCCGAGTAGCTGGGATTACAGGT |
| | | ACCCGCCACCACGCCCAGCTAATTTTTTTGTATTTTTAGTAGAG |
| | | ATGGGGTTTTGCCAAATTGGCCAGGGTGGTCTCAAACTCCTGAC |
| | | CTCAGGTGATCCACCCACCTCGGCCTCCCAAAGTGCTGGGATTA |
| | | CAGGCGTG |
| | | AGCAACCACGTCAAGACAACAATCACTTTCTTTAAAGCAAATCC |
| | | TACAGCTGGTCAACACCCTATTCCATCTGTCATCGAGAAAGAAA |
| | | ATGTTAAAATAGACTTAAAAATATTGCTTTGTTACATATAATAA |
| | | TATGGCATGATGATGTTATTTTTTTCTTAATACTCAAGAAAAAA |
| | | TATATGGTGGTATCTTTTACAACACTGGAACAGAAATAAAGTTT |
| | | CCCTTGAAGGC |

*Underlined sequences indicate CD33 signal peptide (SEQ ID NO: 10).
**Bold sequences indicate coding sequence (CDS).

The present disclosure is based, at least in part, on the discovery that certain modifications to the GFRAL extracellular domain confer functional advantages over wild-type GFRAL extracellular domains in, e.g., GDF15 activity assays and in therapeutic combinations. See, e.g., Examples 3-11. In some embodiments, a GFRAL extracellular domain comprising domains D2 and D3 but lacking domain D1 exhibits increased binding activity to GDF15, as compared to a corresponding GFRAL extracellular domain comprising domain D1. In some embodiments, a GFRAL extracellular domain comprising domains D2 and D3 but lacking domain D1 (in complex with GDF15) exhibits increased potency in RET activation and signaling, as compared to a corresponding GFRAL extracellular domain comprising domain D1 (in complex with GDF15).

The GFRAL D1 domain comprises six N-glycosylation sites, resulting in heterogeneous N-glycosylation and an increase of up to 18 kD in the molecular mass of the full length GFRAL extracellular domain (Goodman et al., Cell Rep 2014; 8(6):1894-1904). The GFRAL D2 domain is capable of binding to GDF15 and interacting with the membrane-proximal cysteine-rich region of the RET extracellular domain. Without wishing to be bound by theory, the presence of the carbohydrates on the GFRAL D1 domain and the folding of the full length GFRAL extracellular domain may mask or inhibit the interaction of the GFRAL D2 domain with GDF15 and the RET extracellular domain.

After the removal of domain D1 from the full length GFRAL extracellular domain, e.g., as described in the examples provided herein, GDF15 and the GFRAL extracellular domain comprising domains D2 and D3 but lacking domain D1 (GFRAL(D2D3)) may form a smaller, more compact complex than GDF15 and the full length GFRAL extracellular domain (GFRAL(ECD)). Without being bound by theory, this smaller, more compact complex may better fit into the pocket formed by dimerization of RET extracellular domains, resulting in increased binding activity of the GFRAL(D2D3)/GDF15 complex to RET. This may lead to increased potency of the GFRAL(D2D3)/GDF15 complex in activation of RET and provide a benefit for both therapeutic and cell-based assay purposes. The larger complex of GFRAL(ECD)/GDF15 may be less stable. Also, upon engagement with RET on the cell surface, the interaction of the larger complex with surface RET may not be as strong as the smaller complex, based on observations in binding assays, and therefore lead to reduced potency in RET activation and signaling.

In some embodiments, the benefits of a GFRAL extracellular domain comprising domains D2 and D3 but lacking domain D1 may provide for enhanced assays to evaluate the potency or efficacy of a GDF15 peptide. In some embodiments, these benefits of a GFRAL extracellular domain comprising domains D2 and D3 but lacking domain D1 may provide for improved therapeutic benefits in treating obesity or related disorders by administering the GFRAL alone or in combination with a GDF15 peptide (e.g., a fatty acid conjugated or albumin fusion GDF15 peptide).

In certain aspects, the disclosure herein features a cell-based assay to detect the activity of a GDF15 peptide, comprising: (a) providing a cell that expresses a cell surface receptor kinase; (b) contacting the cell with the GDF15 peptide and a soluble GFRAL; and (c) detecting a biological response in the contacted cell, wherein the soluble GFRAL comprises a GFRAL extracellular domain comprising domains D2 and D3.

In certain other aspects, the present disclosure features a cell-based assay to detect the activity of a GDF15 peptide, comprising: (a) providing a cell that expresses a GFRAL extracellular domain (e.g., a soluble GFRAL extracellular domain) and a cell surface receptor kinase; (b) contacting the cell with the GDF15 peptide; and (c) detecting a biological response in the contacted cell, wherein the GFRAL extracellular domain comprises domains D2 and D3.

As used herein, “GDF15” and “GDF15 peptide” refers to any GDF15 polypeptide of mammalian origin, or variant or functional fragment thereof. In various embodiments, the GDF15 peptide is a human GDF15 peptide, or a variant or functional fragment thereof. In various embodiments, human GDF15 is synthesized as a 308-amino acid preprotein (SEQ ID NO:12; UniProt Ref. Sequence: Q99988) that includes a signal peptide (amino acids 1-29), a propeptide (amino acids 30-196), and the 112-amino acid mature GDF15 peptide (amino acids 197-308 (also identified as SEQ ID NO:13)) (see, e.g., Bootcov et al., Proc Natl Acad Sci USA 1997; 94(21):11514-9), although the boundaries between these subsequences may vary slightly. For instance, in various embodiments, the human GDF15 preprotein (SEQ ID NO:12; UniProt Ref. Sequence: Q99988) may be subdivided into a signal peptide (amino acids 1-29), a propeptide (amino acids 30-194), and the mature GDF15 peptide (amino acids 195-308). In various embodiments, a mature GDF15 peptide comprises amino acids 197-308 of SEQ ID NO:12 and is identified herein as SEQ ID NO:13. In various other embodiments, a mature GDF15 peptide comprises amino acids 200-308 of SEQ ID NO:12 and is identified herein as SEQ ID NO:14.

In addition, sequence variations have been reported. For example, amino acids 202, 269, and 288 of SEQ ID NO:12 have been reported as Asp, Glu, and Ala, respectively (see, e.g., Hromas et al., Biochim Biophys Acta 1997; 1354(1):40-4; Lawton et al., Gene 1997; 203(1):17-26). An exemplary sequence variant containing the Asp substitution at amino acid 202 (“GDF15 H6D variant”) is described, e.g., in Amaya-Amaya et al., J Immunol Res 2015; 2015:270763.

A variant of a GDF15 peptide, or a functional fragment thereof, can include one or more amino acid deletions, additions and/or substitutions, in any desired combination. The amount of amino acid sequence variation (e.g., through amino acid deletions, additions, and/or substitutions) is limited to preserve activity (e.g., GFRAL signaling activity) of the mature GDF15 peptide. In some embodiments, the variant of a mature GDF15 peptide has from about 1 to about 20, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, or about 1 to about 5 amino acid deletions, additions, or substitutions, in any desired combination, relative to SEQ ID NO:13. Alternatively, or in addition, the variant can have an amino acid sequence that has at least about 80%, at least about 85%, at least about 90%, or at least about 95% amino acid sequence identity with SEQ ID NO:13, when measured over the full length of SEQ ID NO:13. In various embodiments, a GDF15 peptide or variant has at least 80% amino acid sequence identity with the amino acid sequence of SEQ ID NO:13. In various embodiments, a GDF15 peptide or variant has at least 85% amino acid sequence identity with the amino acid sequence of SEQ ID NO:13. In various embodiments, a GDF15 peptide or variant has at least 90% amino acid sequence identity with the amino acid sequence of SEQ ID NO:13. In various embodiments, a GDF15 peptide or variant has at least 95%, 96%, 97%, 98%, or 99% amino acid sequence identity with the amino acid sequence of SEQ ID NO:13.

The GDF15 peptides and variants disclosed herein may also comprise additional modifications, such as affinity tags (e.g., a His tag), PEGylations, glycosylations, fusions (with, e.g., human or mouse serum albumin, an immunoglobulin constant region), and conjugations (with, e.g., fatty acids). See, e.g., the conjugations disclosed in WO 2015/200078, which is incorporated herein by reference; see also the fusions disclosed in WO 2015/198199 and WO 2017/109706, which are both incorporated herein by reference. GDF15 peptides and variants comprising or modified with an affinity tag, a PEGylation, a glycosylation, a fusion, a conjugation, or any additional modification(s) that may support desirable physiologic responses to the peptide (e.g., desirable pK, clearance, half-life, solubility, etc.) are encompassed by the term “GDF15 peptide.”

Without wishing to be bound by theory, it has been reported that GDF15 specifically binds to the GFRAL receptor, and that the GFRAL receptor requires association with the co-receptor RET to elicit intracellular signaling in response to GDF15 stimulation (Yang et al., Nat Med 2017; 23(10):1158-1166). It is also generally known that biologically active GDF15 is a 25-kD homodimer of the mature peptide covalently linked by one interchain disulfide bond. Accordingly, when a GDF15 peptide is a variant of GDF15, any amino acid deletions, additions, and/or substitutions are generally at positions that are not involved with receptor binding or with the peptide-peptide interface. For example, the amino acids at positions 216, 222, 223, 225, 237, 239, 241, 252, 253, 254, 257, 258, 260, 261, 264, 265, 268, 269, 270, 273, 275, 276, 279, 297, 299, 300 and 308 of SEQ ID NO:12 are thought to be involved in the peptide-peptide interface. Any amino acid substitutions at these positions are generally disfavored, and any substitutions are generally conservative substitutions. Amino acids that are surface-exposed but are not conserved among species can, in some embodiments, be substituted with other amino acids without disrupting the folding of the peptide or its activity. Any such variant of GDF15 may be used as a GDF15 peptide in the disclosure herein. Such variants can also be conjugated to a second agent, e.g., a therapeutic agent, detectable label, and/or an agent that supports desirable pK, clearance, half-life, and/or other physiologic responses to the peptide (e.g., histidine tagged, albumin tagged, or fatty acid tagged GDF15 peptide variants). The GDF15 peptides and variants disclosed herein include naturally-occurring and synthetic variants of GDF15. Exemplary variants include but are not limited to: (i) GDF15 H6D variants (see, e.g., Amaya-Amaya et al., J Immunol Res 2015; 2015:270763, which is incorporated herein by reference); (ii) fusions of GDF15 with immunoglobulin constant regions (see, e.g., Xiong et al., Sci Transl Med 2017; 9(412): eaan8732; and WO 2012/138919, which are both incorporated herein by reference); (iii) fusions of GDF15 with alpha-1-antitrypsin (see, e.g., WO 2016/102580, which is incorporated herein by reference); (iv) additions of short peptides to the GDF15 N-terminus and/or C-terminus (see, e.g., WO 2017/202936, which is incorporated herein by reference); (v) sequence variations, e.g., to improve solubility (see, e.g., U.S. Pat. No. 9,161,966, which is incorporated herein by reference); and (vi) PEGylations and/or glycosylations (see, e.g., U.S. Pub. No. US 2015/0023960 A1 and U.S. Pat. No. 9,161,966, which are both incorporated herein by reference).

Additional variants include those disclosed herein (see, e.g., SEQ ID NOs:15-17) and others described in: WO 2013/148117, WO 2014/120619, WO 2015/197446, WO 2015/198199, WO 2016/069921, WO 2016/018931, and WO 2016/102580, which are all incorporated herein by reference. Any GDF15 peptide or variant described in WO 2013/148117, WO 2014/120619, WO 2015/197446, WO 2015/198199, WO 2016/069921, WO 2016/018931, or WO 2016/102580 may be used as a GDF15 peptide in the disclosure herein.

Exemplary GDF15 sequences are set forth in Table 2.

TABLE 2
Exemplary GDF15 sequences.

| | SEQ ID | |
|---|---|---|
| Name | NO | Amino acid sequence* |
| Human His- | 11 | MPLLLLLPLLWAGALAHHHHHHGSGGARNGDHCPLGPGRCCRL |
| GDF15 | | HTVRASLEDLGWADWVLSPREVQVTMCIGACPSQFRAANMHAQ |
| | | IKTSLHRLKPDTVPAPCCVPASYNPMVLIQKTDTGVSLQTYDD |
| | | LLAKDCHCI |
| Human GDF15 | 12 | MPGQELRTVNGSQMLLVLLVLSWLPHGGALSLAEASRASFPGP |
| (preprotein) | | SELHSEDSRFRELRKRYEDLLTRLRANQSWEDSNTDLVPAPAV |
| | | RILTPEVRLGSGGHLHLRISRAALPEGLPEASRLHRALFRLSP |
| | | TASRSWDVTRPLRRQLSLARPQAPALHLRLSPPPSQSDQLLAE |
| | | SSSARPQLELHLRPQAARGRRRARARNGDHCPLGPGRCCRLHT |
| | | VRASLEDLGWADWVLSPREVQVTMCIGACPSQFRAANMHAQIK |
| | | TSLHRLKPDTVPAPCCVPASYN |
| | | PMVLIQKTDTGVSLQTYDDLLAKDCHCI |
| Human GDF15 | 13 | ARNGDHCPLGPGRCCRLHTVRASLEDLGWADWVLSPREVQVTM |
| (mature)- | | CIGACPSQFRAANMHAQIKTSLHRLKPDTVPAPCCVPASYNPM |
| 197-308 | | VLIQKTDTGVSLQTYDDLLAKDCHCI |
| Human GDF15 | 14 | GDHCPLGPGRCCRLHTVRASLEDLGWADWVLSPREVQVTMCIG |
| (mature)- | | ACPSQFRAANMHAQIKTSLHRLKPDTVPAPCCVPASYNPMVLI |
| 200-308 | | QKTDTGVSLQTYDDLLAKDCHCI |
| Human GDF15 | 15 | AHNGDHCPLGPGRCCRLHTVRASLEDLGWADWVLSPREVQVTM |
| (mature)- | | CIGACPSQFRAANMHAQIKTSLHRLKPDTVPAPCCVPASYNPM |
| R198H variant | | VLIQKTDTGVSLQTYDDLLAKDCHCI |
| Human GDF15 | 16 | AHAGDHCPLGPGRCCRLHTVRASLEDLGWADWVLSPREVQVTM |
| (mature)- | | CIGACPSQFRAANMHAQIKTSLHRLKPDTVPAPCCVPASYNPM |
| R198H, N199A | | VLIQKTDTGVSLQTYDDLLAKDCHCI |
| variant | | |
| Human GDF15 | 17 | AREGDHCPLGPGRCCRLHTVRASLEDLGWADWVLSPREVQVTM |
| (mature)- | | CIGACPSQFRAANMHAQIKTSLHRLKPDTVPAPCCVPASYNPM |
| N199E variant | | VLIQKTDTGVSLQTYDDLLAKDCHCI |

*Underlined sequence indicates CD33 signal peptide (SEQ ID NO: 10).

As used herein, “activity” refers to the ability of a GFRAL ligand (e.g., a GDF15 peptide) to effect a change in a biological process. In some embodiments, the activity of a GFRAL ligand is determined by whether it elicits or induces a defined biological response in a cell contacted with the ligand. In some embodiments, the results of a cell-based activity assay are expressed as the “relative activity,” when comparing a test molecule to a reference standard or reference molecule. The use of relative activity allows direct comparison between the molecules to be tested and the reference molecules within the same assay, thus reducing the impact or run-to-run variability on final reportable results.

In cell-based activity assays, a reference molecule is usually used to assign relative activity, ensuring the measurement of activity is normalized over various molecules to be tested. As used herein, “reference molecule” in a GFRAL ligand activity assay refers to a GFRAL ligand with known biological activity. For example, the GFRAL ligand can be a GDF15 peptide with known biological activity. In some embodiments, a reference molecule is a wild-type or a recombinant wild-type GDF15 peptide (e.g., human or recombinant human GDF15). In other embodiments, the reference molecule is a variant of a wild-type or a recombinant wild-type GDF15 peptide (e.g., a variant of human or recombinant human GDF15, wherein the biological activity of the GDF15 variant is already known). In still other embodiments, the reference molecule is a representative batch of GDF15 for therapeutic use. In some embodiments, cells are grown in culture plates and stimulated with the reference molecule and the GFRAL ligand to be tested over a range of concentrations. In some embodiments, the range of concentrations covers the whole dose response range from 0 to a maximal concentration. In some other embodiments, the whole dose response curve is sigmoidal in shape.

As used herein, “biological response” refers to a response in a cell after the cell is contacted with a GFRAL ligand, such as a GDF15 peptide. A biological response can include any response related to, for example, cell signaling or signal transduction (e.g., phosphorylation of a protein kinase), gene transcription, protein expression, toxicity, cytokine release, cell proliferation, cell motility or morphology, cell growth arrest, or cell death (e.g., apoptosis).

In various embodiments, a biological response in a cell after the cell is contacted with a GFRAL ligand, such as a GDF15 peptide, can be evaluated or measured using any of the exemplary assays described herein or known in the art. In various embodiments, the assay involves contacting a cell or culture of cells with a GFRAL ligand (e.g., a GDF15 peptide) and determining whether one or more properties of the cell or culture changes after contact. In various embodiments, a change may be detected in a level of RNA expression, a level of protein expression, a level of protein activity, a level of protein modification (e.g., protein phosphorylation), a level of a reporter signal, toxicity, cytokine release, cell proliferation, cell motility or morphology, cell growth arrest, and/or cell death (e.g., apoptosis).

In some embodiments, the biological response is an increase or decrease in the expression or activity of a protein in the contacted cell, as compared to the expression or activity of the same protein in a control cell that is not contacted with the GFRAL ligand (e.g., a GDF15 peptide). In some embodiments, the biological response is a signal transduction response. In some embodiments, the signal transduction response comprises phosphorylation of a serine, tyrosine, or threonine residue on an intracellular protein. In some embodiments, the signal transduction response comprises phosphorylation of ERK (e.g., ERK1, ERK2). In some embodiments, the signal transduction response comprises phosphorylation of AKT (e.g., AKT1, AKT2, AKT3).

In some embodiments, the biological response is detected using one or more assays to evaluate protein expression, activity, and/or phosphorylation level. In some embodiments, the biological response is detected using one or more assays selected from a kinase or enzymatic activity assay, incubation of whole cells with radiolabeled ³²P-orthophosphate, two-dimensional gel electrophoresis, an immunoblot assay (e.g., Western blot), an AlphaLISA® assay, an enzyme-linked immunosorbent assay (ELISA), a cell-based ELISA assay, intracellular flow cytometry, immunocytochemistry (ICC), immunohistochemistry (IHC), mass spectrometry, multi-analyte profiling (e.g., a phospho-protein multiplex assay), and fluorescent in situ hybridization (FISH).

The term “signal transduction” refers to the biochemical process involving transmission of extracellular stimuli, via cell surface receptors through a specific and sequential series of molecules, to genes in the nucleus resulting in specific cellular responses to the stimuli. Signal transduction is generally part of a communication system that governs cellular activities and coordinates cell actions. Through such communication systems, cells can perceive and respond to changes in extracellular conditions. In various embodiments of the present disclosure, a cell contacted with a GFRAL ligand (e.g., a GDF15 peptide) responds to the presence of the GFRAL ligand by initiating a signal transduction response. In various embodiments, a cell contacted with a GFRAL ligand (e.g., a GDF15 peptide) and a GFRAL receptor polypeptide (e.g., a soluble GFRAL) responds to the presence of the GFRAL ligand and GFRAL receptor polypeptide by initiating a signal transduction response. In various embodiments, the cell is a cell expressing a cell surface receptor kinase, either endogenously or exogenously. In various embodiments, the cell surface receptor kinase is a RET receptor tyrosine kinase. In various embodiments, the cell also expresses an exogenous GFRAL extracellular domain.

GFRAL, an orphan member of the glial cell line-derived neurotrophic factor (GDNF) receptor alpha family, has been identified as a receptor that binds directly to GDF15. However, to elicit intracellular signaling in response to GDF15 stimulation, GFRAL in complex with GDF15 typically binds to and activates a RET cell surface receptor kinase. GDF15 typically does not induce downstream signals in cells expressing GFRAL only or RET only, but GDF15 signals may be detected in cells expressing both GFRAL and RET. Without wishing to be bound to theory, it is believed that the in vivo activity of GDF15 is mediated through both GFRAL and RET by forming a ternary complex. In various embodiments, a GDF15 peptide and a GFRAL receptor polypeptide (e.g., a soluble GFRAL) form a binary complex. In various embodiments, the binary complex binds to RET to form a ternary complex. In various embodiments, the formation of a GDF15-GFRAL-RET ternary complex activates RET and stimulates RET-mediated intracellular signaling.

The term “complex,” as used herein, refers to a non-covalent association between at least two moieties (e.g. chemical or biochemical) that have an affinity for one another. Examples of complexes include the non-covalent association between an antigen/antibody, lectin/avidin, target polynucleotide/probe oligonucleotide, antibody/anti-antibody, receptor/ligand (e.g., GFRAL and GDF15), enzyme/ligand, polypeptide/polypeptide, polypeptide/polynucleotide, polypeptide/co-factor, polypeptide/substrate, polypeptide/inhibitor, polypeptide/small molecule, and the like. The term “binary complex” means the non-covalent association is between two such moieties, such as a receptor and a ligand (e.g., GFRAL and GDF15). The term “ternary complex” means the non-covalent association is between three moieties, such as a receptor, a co-receptor, and a ligand (e.g., GFRAL, GDF15, and RET).

As used herein, “RET” refers to a RET receptor polypeptide having the amino acid sequence of any naturally-occurring full length RET receptor polypeptide, or any variant or functional fragment thereof which is capable of binding to and being activated by a GFRAL when in complex with GDF15. In some embodiments, the RET is a full length mammalian RET (e.g., human, monkey, rat, or mouse RET), or a variant or functional fragment thereof. In some embodiments, the RET is full length human RET.

“RET” is an abbreviation for “rearranged during transfection,” as the DNA sequence of the RET gene was originally found to be rearranged within a 3T3 fibroblast cell line following its transfection with DNA taken from human lymphoma cells. The natural alternative splicing of the human RET gene results in the production of 3 different isoforms of the protein RET: RET51, RET43, and RET9. These three isoforms share the same 1063 amino acids at their N-terminus, but then contain 51, 43, and 9 different amino acids at their cytoplasmic C-terminus, respectively. All three isoforms are encompassed by the term “RET” herein. Typical of receptor tyrosine kinases, RET has an extracellular domain, a transmembrane domain, and an intracellular kinase domain. See, e.g., Mulligan, Nat Rev Cancer 2014; 14(3):173-86.

In various embodiments, RET activation occurs when RET is bound by a GFRAL in complex with GDF15. In various embodiments, RET activation occurs when GDF15, GFRAL, and RET form a ternary complex (i.e., a GDF15-GFRAL-RET ternary complex).

In various embodiments, RET activation comprises RET dimerization and phosphorylation of certain residues in the RET intracellular kinase domain. Such phosphorylated residues in the RET intracellular domain may facilitate direct interactions with signaling molecules such as phospholipase C gamma (PLCγ) or with various adaptor proteins, which may lead to the activation of multiple downstream signaling pathways.

The activity of a GFRAL ligand (e.g., a GDF15 peptide) is determined, in various embodiments, by whether it elicits or induces a defined biological response in a cell contacted with the ligand. In some embodiments, the defined biological response is a signal transduction response. In some embodiments, the signal transduction response involves activation of one or more of the ERK/MAPK pathway, the PI3K/AKT pathway, the protein kinase C pathway, the JAK/STAT pathway, the JNK pathway, the p38 pathway, and the RAC1 pathway, which can be measured according to methods known in the art.

One major mechanism for signal transduction involves protein phosphorylation. In some embodiments, the signal transduction response is an increase or decrease in phosphorylation of a protein kinase in the cell that is contacted with a GFRAL ligand (e.g., a GDF15 peptide), as compared to phosphorylation of the same protein kinase in a control cell that is not contacted with the GFRAL ligand.

As used herein, “protein kinase” refers to an enzyme that transfers a phosphate group from a phosphate donor onto an acceptor amino acid in a substrate protein. Protein kinases may be classified based on the acceptor amino acid specificity. The two most well characterized types of protein kinases are protein serine/threonine kinases (a protein kinase with a protein alcohol group as acceptor) and protein tyrosine kinases (a protein kinase with a protein phenolic group as acceptor). RET, as well as all protein kinases that are directly or indirectly phosphorylated by an activated RET, are intended to be encompassed by the term “protein kinase.” Exemplary protein kinases are described herein, and others are known in the art. RET receptor interactions and signal transduction pathways are reviewed, e.g., in Mulligan, Nat Rev Cancer 2014; 14(3):173-86.

In some embodiments, the protein kinase is an intracellular protein kinase of the ERK/MAPK pathway (also referred to herein as the “RET-ERK” pathway). In some embodiments, the protein kinase is selected from one or more of ERK (ERK1, ERK2), JAK1, JAK2, RAF, MEK1, MEK2, RSK1, RSK2, RSK3, MNK1, MNK2, MSK1, and MSK2, or any downstream targets thereof. In some embodiments, the protein kinase is selected from one or more of ERK (ERK1, ERK2), JAK1, JAK2, RAF, MEK1, MEK2, RSK1, RSK2, RSK3, MNK1, MNK2, MSK1, and MSK2. Exemplary intracellular proteins in the RET-ERK pathway include ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2, as well as any upstream modulators and downstream targets thereof.

In some embodiments, a signal transduction response in a cell that is contacted with a GFRAL ligand (e.g., a GDF15 peptide) may be an increase or decrease in the expression or activity of any intracellular protein in the RET-ERK pathway, as compared to the expression or activity of the same protein in a control cell that is not contacted with the GFRAL ligand.

In some embodiments, the protein kinase is an intracellular protein kinase of the PI3K/AKT pathway (also referred to herein as the “RET-AKT” pathway). In some embodiments, the protein kinase is selected from one or more of AKT (AKT1, AKT2, AKT3), SRC, JAK1, JAK2, PI3K, PDK1, MLK3, ASK1, GSK3alpha, GSK3beta, and mTOR, or any downstream targets thereof. In some embodiments, the protein kinase is selected from one or more of AKT (AKT1, AKT2, AKT3), SRC, JAK1, JAK2, PI3K, PDK1, MLK3, ASK1, GSK3alpha, GSK3beta, and mTOR. In some embodiments, the downstream target in the RET-AKT pathway is S6 kinase. Exemplary intracellular proteins in the RET-AKT pathway include AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR, as well as any upstream modulators and downstream targets thereof. In some embodiments, a signal transduction response in a cell that is contacted with a GFRAL ligand (e.g., a GDF15 peptide) may be an increase or decrease in the expression or activity of any intracellular protein in the RET-AKT pathway, as compared to the expression or activity of the same protein in a control cell that is not contacted with the GFRAL ligand.

In some embodiments, the protein kinase is an intracellular protein kinase of the protein kinase C pathway. In some embodiments, the protein kinase is protein kinase C. In some embodiments, RET activation comprises phosphorylation of phospholipase C gamma (PLCγ). In some embodiments, phosphorylated PLCγ activates the protein kinase C pathway. See, e.g., Mullican et al., Nat Med 2017; 23(10):1150-7. In some embodiments, a signal transduction response in a cell that is contacted with a GFRAL ligand (e.g., a GDF15 peptide) may be an increase or decrease in the expression or activity of any intracellular protein in the protein kinase C pathway, as compared to the expression or activity of the same protein in a control cell that is not contacted with the GFRAL ligand.

In some embodiments, the protein kinase is an intracellular protein kinase of the JAK/STAT pathway. In some embodiments, the protein kinase is selected from one or more of JAK1 and JAK2. Exemplary intracellular proteins in the JAK/STAT pathway include JAK1, JAK2, and STAT3, which have been implicated in RET signaling (see, e.g., Mulligan, Nat Rev Cancer 2014; 14(3):173-86), as well as JAK3, TYK2, STAT1, STAT2, and STATS. In some embodiments, a signal transduction response in a cell that is contacted with a GFRAL ligand (e.g., a GDF15 peptide) may be an increase or decrease in the expression or activity of any intracellular protein in the JAK/STAT pathway, as compared to the expression or activity of the same protein in a control cell that is not contacted with the GFRAL ligand.

In some embodiments, the protein kinase is an intracellular protein kinase of the JNK pathway. In some embodiments, the protein kinase is selected from one or more of JNK1, JNK2, TAK1, MKK4, and MKK7. Exemplary intracellular proteins in the JNK pathway include JNK1, JNK2, TAK1, MKK4, and MKK7. In some embodiments, a signal transduction response in a cell that is contacted with a GFRAL ligand (e.g., a GDF15 peptide) may be an increase or decrease in the expression or activity of any intracellular protein in the JNK pathway, as compared to the expression or activity of the same protein in a control cell that is not contacted with the GFRAL ligand.

In some embodiments, the protein kinase is an intracellular protein kinase of the p38 pathway. In some embodiments, the protein kinase is selected from one or more of MKK3, MKK6, a p38 MAPK (e.g., MAPK11, MAPK12, MAPK13, and MAPK 14), MSK1, MSK2, MK2, MK3, MNK1, and MNK2. Exemplary intracellular proteins in the p38 pathway include MKK3, MKK6, a p38 MAPK (e.g., MAPK11, MAPK12, MAPK13, and MAPK 14), MSK1, MSK2, MK2, MK3, MNK1, and MNK2. In some embodiments, a signal transduction response in a cell that is contacted with a GFRAL ligand (e.g., a GDF15 peptide) may be an increase or decrease in the expression or activity of any intracellular protein in the p38 pathway, as compared to the expression or activity of the same protein in a control cell that is not contacted with the GFRAL ligand.

In some embodiments, the protein kinase is an intracellular protein kinase of the RAC1 pathway. In some embodiments, the protein kinase is PKN2. Exemplary intracellular proteins in the RAC1 pathway include RAC1 and PKN2. In some embodiments, a signal transduction response in a cell that is contacted with a GFRAL ligand (e.g., a GDF15 peptide) may be an increase or decrease in the expression or activity of any intracellular protein in the RAC1 pathway, as compared to the expression or activity of the same protein in a control cell that is not contacted with the GFRAL ligand.

In some embodiments, the protein kinase is the cell surface receptor kinase. In some embodiments, the protein kinase and/or cell surface receptor kinase is a RET receptor tyrosine kinase.

Various assays have been developed for measuring protein phosphorylation. The discovery of phospho-specific antibodies against phosphorylated tyrosine/serine/threonine residues or against a specific phosphorylated protein kinase enables immunology assays measuring phosphorylation of protein kinases, such as an immunoblot assay (e.g., Western blot), an AlphaLISA® assay, and an enzyme-linked immunosorbent assay (ELISA). Some other assays measure the ability of a serine/threonine kinase or tyrosine kinase to phosphorylate a synthetic substrate polypeptide (see, e.g., Pike, Methods Enzymol 1987; 146:353-62; Hunter, J Biol Chem 1982; 257(9):4843-8; Wang et al., J Biol Chem 1992; 267(24):17390-6). Such assays can use radioactive labels.

In certain aspects of the present disclosure, the cell contacted with a GFRAL ligand is a cell expressing a cell surface receptor kinase, either endogenously or exogenously via transfection with a construct or vector. In certain aspects, the cell also expresses an exogenous GFRAL extracellular domain. In some embodiments, the cell does not express endogenous GFRAL or an endogenous GFRAL extracellular domain. In some embodiments, the cell does not express endogenous GDF15. In some embodiments, the cell is a GDF15 KO cell comprising an operative GDF15 gene. In some embodiments, the cell is transfected with a construct or vector to exogenously express a GFRAL extracellular domain. Exemplary cells include animal cells. In some embodiments, the animal cell is originated from a mammalian animal, e.g., a human, a primate, or a rodent. In some embodiments, the cell is a human cell. In some embodiments, the cell is an MCF7 cell, an SH-SY5Y cell, or an HEK293A-GDF15 KO cell.

As used herein, “expression” refers to the transcription and translation of a nucleic acid molecule by a cell.

The term “construct,” as used herein, refers to a nucleic acid molecule that has been generated by human intervention, including by recombinant means or direct chemical synthesis, with a series of specified nucleic acid elements that permit transcription and/or translation of a particular nucleic acid in a cell. Constructs can be part of a plasmid, virus, or nucleic acid fragment. Constructs can also include integratable DNA fragments (i.e., fragments integratable into the host genome by genetic recombination) and other vehicles which enable the integration of DNA fragments comprising a gene or a nucleic acid sequence of interest. In some embodiments, the construct comprises control elements and a gene or nucleic acid sequence encoding a RET cell surface receptor kinase. In some embodiments, the construct comprises control elements and a gene or nucleic acid sequence encoding a GFRAL extracellular domain. Exemplary control elements include, but are not limited to, a promoter system, a regulatory element to control the level of mRNA expression, a sequence encoding a ribosome binding site, and a sequence terminating transcription and translation.

The term “endogenous,” as used herein, refers to substances originating or produced within an organism. An “endogenous” gene or protein is a gene or protein residing in a species that is also derived from that species.

The term “exogenous,” as used herein, refers to a substance or molecule originating or produced outside of an organism. An exogenous gene may be from a different species (a “heterologous” gene) or from the same species (a “homologous” gene), relative to the cell being transfected. A transfected cell may be referred to as a recombinant cell.

The term “recombinant,” as used herein, refers to a polynucleotide or polypeptide that does not naturally occur in a host cell. A recombinant molecule may contain two or more naturally-occurring sequences that are linked together in a way that does not occur naturally. A recombinant cell contains a recombinant polynucleotide or polypeptide.

The GFRAL ligands and GFRAL extracellular domains described herein may be produced, in various embodiments, using recombinant expression methods. Recombinant protein expression using a host cell is used routinely in the art. As used herein, the term “host cell” refers to a cell artificially engineered to comprise nucleic acids encoding the sequence of a peptide and which will transcribe and translate, and optionally, secrete the peptide into the cell growth medium. For recombinant production purposes, a nucleic acid encoding the amino acid sequence of the peptide would typically be synthesized or cloned by conventional methods and integrated into an expression vector. Exemplary host cells include but are not limited to Chinese hamster ovary (CHO) cells, human embryonic kidney (HEK) cells (e.g., HEK293, HEK293T, HEK293F, HEK293S), monkey kidney (COS) cells (e.g., COS-1, COS-7), baby hamster kidney (BHK) cells (e.g., BHK-21), African green monkey kidney cells (e.g. BSC-1), HeLa cells, human hepatocellular carcinoma cells (e.g., Hep G2), myeloma cells (e.g., NSO, 653, SP2/0), lymphoma cells, E. coli or other bacterial cells, yeast cells, insect cells, and plant cells, or any derivative, immortalized, or transformed cell thereof. In some embodiments, the host cell is a CHO cell. In some embodiments, the host cell is a HEK cell. In some embodiments, the host cell is a HEK293T, HEK293F, or HEK293S cell. In some embodiments, a HEK293S cell can produce a recombinant protein (e.g., a recombinant GFRAL ligand or GFRAL extracellular domain) with an altered glycosylation pattern (e.g., shorter glycosylation chains).

### Therapeutic Methods and Compositions

GDF15 peptides evaluated using the novel GFRAL receptor polypeptides and cell-based activity assays described herein can be employed in many therapeutic or prophylactic applications. These include, but are not limited to, decreasing adiposity and treating obesity, preventing the development of obesity, decreasing body weight, reversing or slowing of weight gain, decreasing appetite, decreasing feed efficiency, and treating metabolic diseases. Accordingly, the present disclosure provides methods of treating obesity and obesity-related conditions by administering a GDF15 peptide alone or in combination with a GFRAL receptor polypeptide (e.g., a soluble GFRAL). The present disclosure further provides methods of reducing appetite and/or reducing body weight, e.g., in an overweight or obese subject, by administering a GDF15 peptide alone or in combination with a GFRAL receptor polypeptide (e.g., a soluble GFRAL). Uses of a GDF15 peptide alone or in combination with a GFRAL receptor polypeptide, e.g., in treating obesity, reducing appetite, and/or reducing body weight, are also provided. The therapeutic methods and uses provided herein may be useful in the treatment or prevention of obesity, and/or any of a variety of disorders and conditions related to excess body weight.

As used herein, the term “treat” and its cognates refer to an amelioration of a disease, disorder, or condition (e.g., heart failure), or at least one discernible symptom thereof. In some embodiments, “treat” refers to an amelioration of at least one measurable physical parameter, not necessarily discernible by the patient. In some embodiments, “treat” refers to inhibiting the progression of a disease, disorder, or condition, either physically (e.g., stabilization of a discernible symptom), physiologically (e.g., stabilization of a physical parameter), or both. In some embodiments, “treat” refers to slowing the progression or reversing the progression of a disease, disorder, or condition. As used herein, “treat” and its cognates also encompass delaying the onset or reducing the risk of acquiring a given disease, disorder, or condition.

The terms “subject” and “patient” are used interchangeably herein to refer to any human or non-human animal. Non-human animals include all vertebrates (e.g., mammals and non-mammals) such as any mammal. Non-limiting examples of mammals include humans, mice, rats, rabbits, dogs, monkeys, and pigs. In preferred embodiments, the subject is a human.

Subjects who are overweight or obese are at increased risk for a variety of metabolic diseases and serious health problems. These often appear first as part of the metabolic syndrome, which is characterized by elevated blood pressure, high blood sugar, excess body fat around the abdomen, and abnormal blood cholesterol levels. Serious health problems can then develop, such as type II diabetes, hypertension, coronary heart disease, stroke, cancer, osteoarthritis, sleep apnea, dyslipidemia, elevated insulin (insulin resistance), and hypoventilation syndrome. Type II diabetes can also give rise to several other serious health problems, such as diabetic neuropathy, diabetic nephropathy, and diabetic retinopathy. Subjects in need of therapy using an a GDF15 peptide alone or in combination with a GFRAL receptor polypeptide (e.g., a soluble GFRAL) are generally overweight or obese. Generally, an adult human is considered to be overweight if the adult has a body mass index (BMI) (a measurement obtained by dividing a person's weight in kilograms by the square of the person's height in meters) between 25 and 29.9, and an adult human is considered to be obese if the adult has a BMI of 30 or higher. However, this guideline can be adjusted to account for ethnic differences. For example, with ethnic adjustment, an Asian individual having a BMI of 27.5 or higher can be considered obese (WHO Expert Consultation, Lancet 2004; 363(9403):157-63). Subjects who are at increased risk of developing a metabolic disease are also candidates for therapy using a GDF15 peptide alone or in combination with a GFRAL receptor polypeptide (e.g., a soluble GFRAL). For example, subjects with pre-diabetes or an elevated fasting blood glucose level of 100 to 125 mg/dL are candidates for therapy, as are subjects with type II diabetes (those with fasting blood glucose levels of 126 mg/dL or higher).

In certain aspects, the present disclosure relates to methods of treating obesity or an obesity-related disorder in a subject. The term “obesity,” as used herein, refers to conditions in which excess body fat has accumulated to the extent that it may have a negative effect on health, which can, in turn, lead to reduced life expectancy and/or increased health problems. In some instances, a subject can be considered obese when their body mass index (BMI) is greater than 20 kg/m², 21 kg/m², 22 kg/m², 23 kg/m², 24 kg/m², 25 kg/m², 26 kg/m², 27 kg/m², 28 kg/m², 29 kg/m², or 30 kg/m². In some instances, obesity can also be characterized by one or more of fasting glucose levels of at least 100 mg/dL, plasma triglyceride levels of at least 150 mg/dL, HDL cholesterol below 40 mg/dL in men and below 50 mg/dL in women, blood pressure of at least 130/85 mmHg, and abdominal waist circumference of greater than 40 inches for men and greater than 35 inches for women.

The term “obesity-related disorder” refers to any condition that can coincide with obesity or may be a direct or indirect result of having excess body weight. The term encompasses, for example, cancers, body weight disorders, in addition to metabolic diseases and disorders. In some embodiments, the obesity-related disorder is a cancer, a body weight disorder, and/or a metabolic disease or disorder. Exemplary obesity-related disorders are described herein, such as, for example, cancer, type II diabetes mellitus (T2DM), nonalcoholic steatohepatitis (NASH), hypertriglyceridemia, and cardiovascular disease.

As used herein, the term “body weight disorder” refers to conditions associated with excessive body weight and/or enhanced appetite. Various parameters are used to determine whether a subject is overweight compared to a reference healthy individual, including the subject's age, height, sex, and health status. For example, a subject may be considered overweight or obese by assessment of the subject's BMI. In some instances, an adult having a BMI in the range of 18.5 to 24.9 kg/m² may be considered to have a normal weight; an adult having a BMI between 25 and 29.9 kg/m² may be considered overweight (pre-obese); an adult having a BMI of 30 kg/m² or higher may be considered obese. Enhanced appetite frequently contributes to excessive body weight. There are several conditions associated with enhanced appetite, including, for example, night eating syndrome, which is characterized by morning anorexia and evening polyphagia often associated with insomnia, but which may be related to injury to the hypothalamus.

The term “metabolic diseases,” and terms similarly used herein, includes but is not limited to obesity, type II diabetes mellitus (T2DM), pancreatitis, dyslipidemia, nonalcoholic steatohepatitis (NASH), insulin resistance, hyperinsulinemia, glucose intolerance, hypertriglyceridemia, hyperglycemia, metabolic syndrome, hypertension, cardiovascular disease, atherosclerosis, peripheral arterial disease, stroke, heart failure, coronary heart disease, diabetic complications (including but not limited to chronic kidney disease), neuropathy, gastroparesis and other metabolic disorders.

The term “metabolic disease or disorder” refers to an associated cluster of traits that includes, but is not limited to, hyperinsulinemia, abnormal glucose tolerance, obesity, redistribution of fat to the abdominal or upper body compartment, hypertension, dyslipidemia characterized by high triglycerides, low high density lipoprotein (HDL) particles, and high low density lipoprotein (LDL) particles. Subjects having metabolic disease or disorder are at risk for development of T2DM and, for example, atherosclerosis.

As used herein, the term “metabolic syndrome” refers to a cluster of risk factors that raises the risk for heart disease and other diseases like diabetes and stroke. These risk factors include but are not limited to abdominal fat (i.e., in most men a waist to hip ratio>0.9 or BMI>30 kg/m²); high blood sugar (i.e., at least 100 mg/dL after fasting); high triglycerides (i.e., at least 150 mg/dL in the bloodstream); low HDL (i.e., less than 40 mg/dL in men and less than 50 mg/dL in women); and blood pressure of 130/85 mmHg or higher (World Health Organization).

In certain aspects, the present disclosure also relates to methods of treating genetic obesity in a subject, such as Prader-Willi syndrome, leptin mutations and/or melanocortin 4 receptor mutations.

An exemplary embodiment is a method of treating obesity or an obesity-related disorder by administering a GDF15 peptide to a subject, wherein the GDF15 peptide induces a biological response in a cell contacted with the GDF15 peptide and/or has GFRAL signaling activity, as determined using the detection methods described herein. The GDF15 peptide may be administered alone or in combination with a second agent (e.g., a GFRAL receptor polypeptide, e.g., a soluble GFRAL), and may be administered in any acceptable formulation, dosage, and dosing regimen.

Another exemplary embodiment is a method of treating obesity or an obesity-related disorder, comprising administering a GDF15 peptide in combination with a GFRAL to a subject, wherein the GDF15 peptide induces a biological response in a cell contacted with the GDF15 peptide and/or has GFRAL signaling activity, as determined using the detection methods described herein, and wherein the GFRAL comprises a GFRAL extracellular domain comprising domains D2 and D3. In some embodiments, the GFRAL is a soluble GFRAL. In some embodiments, the GFRAL comprises a GFRAL extracellular domain lacking domain D1. In some embodiments, the GFRAL further comprises a signal peptide. In some embodiments, the GFRAL comprises the amino acid sequence of SEQ ID NO:1 or a functional variant thereof. In some embodiments, the GFRAL comprises the amino acid sequence of SEQ ID NO:2 or a functional variant thereof.

Administered “in combination” or “co-administration,” as used herein, means that two or more different treatments are delivered to a subject during the subject's affliction with a medical condition (e.g., obesity). For example, in some embodiments, the two or more treatments are delivered after the subject has been diagnosed with a disease or disorder, and before the disease or disorder has been cured or eliminated. In some embodiments, the delivery of one treatment is still occurring when the delivery of the second treatment begins, so that there is overlap. In some embodiments, the first and second treatment are initiated at the same time. These types of delivery are sometimes referred to herein as “simultaneous,” “concurrent,” or “concomitant” delivery. In other embodiments, the delivery of one treatment ends before delivery of the second treatment begins. This type of delivery is sometimes referred to herein as “successive” or “sequential” delivery. In some embodiments, the GDF15 peptide and the GFRAL are administered simultaneously. In some other embodiments, the GDF15 and the GFRAL are administered sequentially.

In some embodiments of simultaneous administration, the two treatments (e.g., a GDF15 peptide and a GFRAL) are comprised in the same formulation. Such formulations may be administered in any appropriate form and by any suitable route. In some embodiments, the two treatments (e.g., a GDF15 peptide and a GFRAL) are comprised in a mixture. In some embodiments, the two treatments (e.g., a GDF15 peptide and a GFRAL) are in a complex. In some embodiments, the two treatments (e.g., a GDF15 peptide and a GFRAL) are in a binary complex. In some embodiments, the two treatments comprise a GDF15 peptide and a GFRAL. In some embodiments, the GFRAL (e.g., a soluble GFRAL) comprises a GFRAL extracellular domain comprising domains D2 and D3 but lacking domain D1.

In other embodiments of simultaneous administration, the two treatments (e.g., a GDF15 peptide and a GFRAL) are administered as separate formulations, in any appropriate form and by any suitable route. In some embodiments, the two treatments comprise a GDF15 peptide and a GFRAL. In some embodiments, for example, the GDF15 peptide and GFRAL may be administered concurrently, or sequentially in any order at different points in time; in either case, they should be administered sufficiently close in time so as to provide the desired therapeutic or prophylactic effect. In some embodiments, the GFRAL (e.g., a soluble GFRAL) comprises a GFRAL extracellular domain comprising domains D2 and D3 but lacking domain D1.

Conventional pharmaceutical practice is employed to provide suitable formulations or compositions comprising a GDF15 antibody peptide and/or a GFRAL receptor polypeptide (e.g., a soluble GFRAL), and to administer such compositions to subjects or experimental animals. Methods of formulating pharmaceutical compositions are known in the art (see, e.g., “Remington's Pharmaceutical Sciences,” Mack Publishing Co., Easton, Pa.). Appropriate formulation depends on the route of administration.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1: Recombinant Soluble GFRAL Extracellular Domains Produced for GDF15 Binding Assays

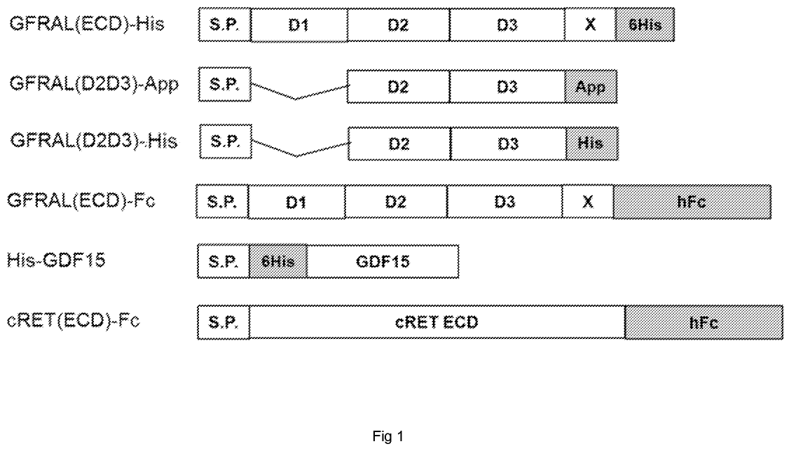

Methods: Human GFRAL(D2D3)-App (SEQ ID NO:3), human GFRAL(D2D3)-His (SEQ ID NO:25), human GFRAL(ECD)-His (SEQ ID NO:6), and human GFRAL(ECD)-Fc (SEQ ID NO:7) gene constructs (FIG. 1) were created according to gene nucleotide and protein sequences of human GFRAL from GenBank and UniProt databases (NCBI Ref. Sequence: NM_207410.2; UniProt Ref. Sequence: Q6UXV0). The sequences were analyzed for signal peptide (S.P.) and functional domains by using Vector NTi software and Blast. Gene constructs containing the full length human GFRAL extracellular domain (GFRAL(ECD)), the D2D3 region of the human GFRAL extracellular domain (GFRAL(D2D3)), and purification tags such as His (six histidines), App (amyloid beta precursor protein), or Fc (human IgG1 Fc), were designed, synthesized, and cloned into a pRS5a expression vector backbone under the control of a CMV promoter for gene expression in HEK293T or HEK293F cells or in CHO cells. Human cRET(ECD)-Fc (SEQ ID NO:8) (FIG. 1) was created by fusion of human RET extracellular domain (RET-ECD) with human IgG1 Fc. The human CD33 signal peptide (SEQ ID NO:10) was fused to the N-terminus of each construct to direct protein secretion.

An additional construct, His-GDF15, was designed to encode the N-terminally six histidine-tagged human GDF15 (SEQ ID NO:11) (FIG. 1). The construct was synthesized and cloned in a pRS5a expression vector backbone for co-transfection and co-purification of a GDF15-derived GFRAL complex.

New constructs:
Human GFRAL(D2D3)-App
Human GFRAL(D2D3)-His
Human GFRAL(ECD)-His
Human GFRAL(ECD)-Fc
Human cRET(ECD)-Fc
Human His-GDF15

Cell lines: Human embryonic kidney cell suspension cell lines HEK293T or HEK293F or CHO cells were propagated in free style 293 expression medium (FS293) at 37° C. for transient transfection and generation of recombinant GFRAL(D2D3)-App, GFRAL(D2D3)-His, GFRAL(ECD)-His, GFRAL(ECD)-Fc, cRET(ECD)-Fc, and other proteins or protein complexes of the present disclosure.

Reagents: PureLink Expi endotoxin-free Giga plasmid purification kits (ThermoFisher Scientific, Waltham Mass.) were used to generate endotoxin-free plasmid DNAs for transfection and protein production. Polyethylenimine solution (PEI) was used for transfection. Ni-NTA super flow cartridges (Qiagen, Germantown Md.) and HiTrap Protein A columns (GE Healthcare Life Sciences, Marlborough Mass.) were used for protein purification.

Expression and purification of recombinant proteins: To generate purified recombinant GFRAL and cRET proteins, as well as the co-expressed complexes of these proteins with His-GDF15, expression vectors were diluted into FS293 medium and mixed with PEI solution at a ratio of 1:2.5 (w/w) to form DNA/PEI complex prior to addition to HEK293T or HEK293F cell cultures or CHO cell cultures accordingly. For instance, 1 mg expression plasmid DNA was complexed with 2.5 mg PEI to transiently transfect 1-liter HEK293 cell culture. 4 days post-transfection, supernatants were harvested from transfected cell cultures, filtered, and run through appropriate affinity columns to purify recombinant proteins. His-tagged proteins were purified through Qiagen Ni-NTA super flow cartridges, App-tagged proteins through Sepharose resins conjugated with anti-App mAb (monoclonal antibody), and Fc fusions through Protein A columns. Proteins bound to nickel columns were eluted with 350 mM imidazole. For the GFRAL(ECD)-His construct alone, additional purification of monomeric species was performed by size exclusion chromatography through a Superdex200 column (GE Healthcare Life Sciences, Marlborough Mass.) before use in experiments. Proteins bound to anti-App mAb-conjugated Sepharose resins or Protein A columns were eluted with 50 mM citric acid solution (pH 3.0) supplemented with 150 mM NaCl, followed by neutralization with 1 M Tris HCL. Subsequently, the eluted protein fractions were buffer exchanged and concentrated in Dulbecco's phosphate buffered saline (DPBS). Purified recombinant His-GDF15 was produced using E. coli as described in US 2017/204149 and was either biotinylated or used as-is for in vitro reconstitution with recombinant GFRAL ECD proteins for plate-based cRET binding and cellular assays.

### Example 2: Co-Expression and Co-Purification of Soluble GFRAL Extracellular Domain Proteins with GDF15

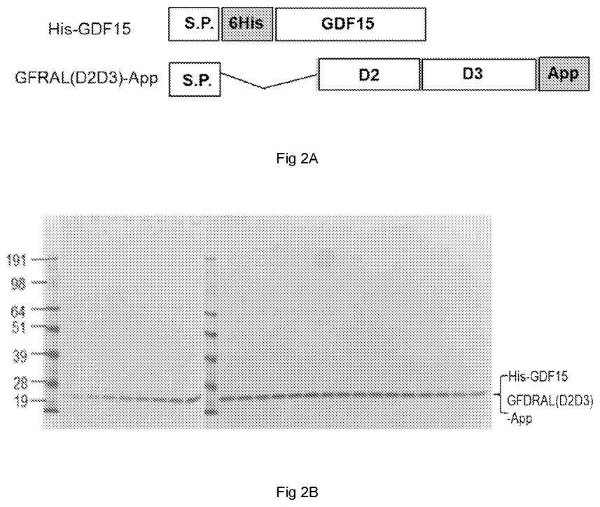

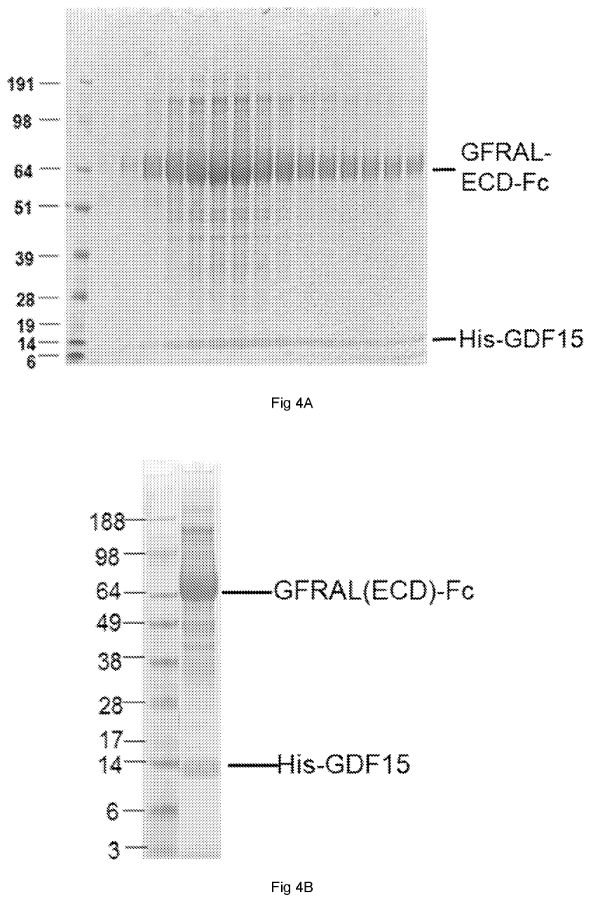

Methods: To generate GFRAL(D2D3)-App/His-GDF15 and GFRAL(ECD)-Fc/His-GDF15 complexes, His-GDF15 vector DNA was mixed with an equal amount of hGFRAL(D2D3)-App vector or hGFRAL(ECD)-Fc vector for transient transfection of 3-liter HEK293T or HEK293F cultures using PEI methods as described in Example 1. 4 days post-transfection, GFRAL(D2D3)-App/His-GDF15 complex (FIGS. 2A-B and 3) and GFRAL(ECD)-Fc/His-GDF15 complex (FIGS. 4A-B) were purified through Ni-NTA cartridges and Protein A columns, respectively. The eluted protein fractions were buffer-exchanged, concentrated in DPBS, and analyzed by SDS-PAGE electrophoresis, size exclusion, and mass spectrometry:

GFRAL(D2D3)-App/His-GDF15 complex was purified from 3000 ml culture medium (elution profile not shown). FIG. 2A shows exemplary His-GDF15 and GFRAL(D2D3)-App constructs. FIG. 2B shows screening of fractions by SDS-PAGE analysis. Single protein bands shown in FIG. 2B contain both GFRAL(D2D3)-App monomer (24.6 kD) and His-GDF15 dimer (26.6 kD for dimer, 13.3 kD for monomer).

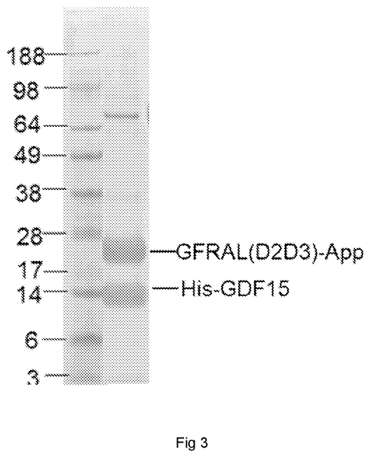

Co-expressed GFRAL(D2D3)-App/His-GDF15 complex was analysed as follows: The complex concentrated from several fractions contains co-expressed GFRAL(D2D3)-App and His-GDF15, as revealed by SDS-PAGE under reducing conditions (FIG. 3). The complex contains 24.6 kD GFRAL(D2D3)-App and 13.3 kD His-GDF15. Co-expressed GFRAL(D2D3)-App/His-GDF15 complex (20 μg) was further analyzed by size exclusion (data not shown). The peak indicates the presence of GFRAL(D2D3)-App/His-GDF15 complex. The molecular masses for GFRAL(D2D3)-App and His-GDF15 in the binary complex were also analyzed under reducing conditions. A 24355 Dalton peak is GFRAL(D2D3)-App polypeptide from which a glycine was clipped from the N-terminus, and an aspartic acid and a serine were clipped from the C-terminus, during the purification process.

GFRAL(ECD)-Fc/His-GDF15 complex was affinity purified from a Protein A column. FIG. 4A shows fractions that contain His-GDF15/GFRAL(ECD)-Fc complex, analyzed by SDS-PAGE under non-reducing conditions. FIG. 4B shows that the complex concentrated from fractions in FIG. 4A contains His-GDF15 and GFRAL(ECD)-Fc, as revealed by SDS-PAGE under reducing conditions.

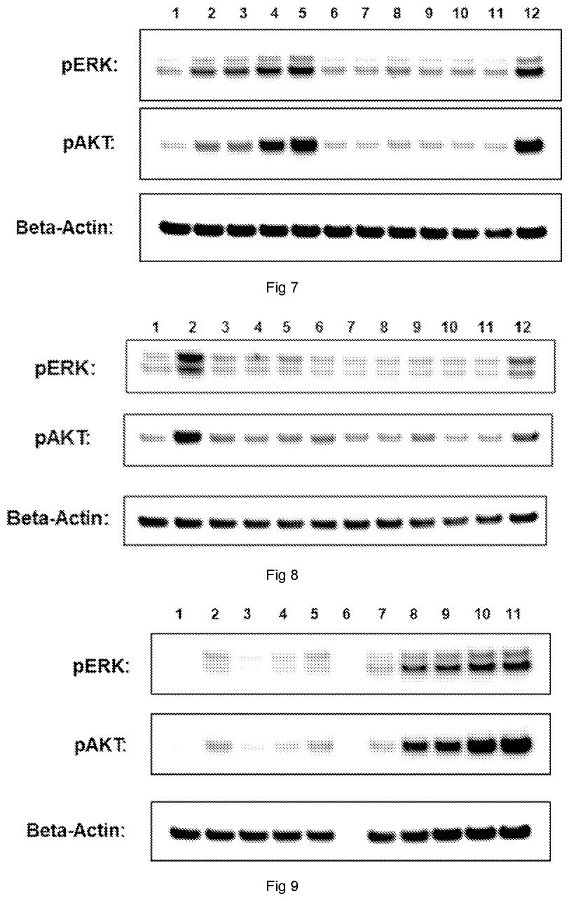

The purifications yielded 1.6 mg GFRAL(D2D3)-App/His-GDF15 and 14 mg GFRAL(ECD)-Fc/His-GDF15 complexes. These were further utilized in cRET-expressing cell activation assays (FIGS. 7 and 8).

### Example 3: GDF15 Complexes with Soluble Recombinant GFRAL Variants Bind to RET-Fc Protein-Coated Plates

Biotinylated recombinant His-GDF15 (biotin) was mixed with purified full length GFRAL(D2D3)-App, GFRAL(ECD)-His, or GFRAL(ECD)-Fc polypeptides (prepared as described in Example 1) to form binary molecular complexes. Complexes were then diluted and incubated with plates coated with recombinant cRET(ECD)-Fc.

Methods: To generate soluble GDF15/GFRAL complexes in vitro, recombinant GFRAL(D2D3)-App, GFRAL(ECD)-His, and GFRAL(ECD)-Fc proteins purified in Example 1 were freshly mixed with equimolar amounts of biotinylated His-GDF15 (1250 pM) in DPBS supplemented with 50 μM CaCl₂. The mixtures were incubated at room temperature for 60 min to allow complex formation. The complex made was stable at 4° C. and was used directly for in vitro binding assays against cRET(ECD)-Fc coated on plastic plates without further purification. Three different GDF15/GFRAL ECD complexes were prepared, including His-GDF15(biotin)/GFRAL(D2D3)-App, His-GDF15(biotin)/GFRAL (ECD)-His, and His-GDF15(biotin)/GFRAL (ECD)-Fc in the same manner. Purified recombinant His-GDF15(L294R), a non-functional mutant in which the leucine residue at position 294 was replaced with an arginine (see US 2017/204149) was prepared in the same way as wild-type His-GDF15. His-GDF15(L294R) was mixed with GFRAL(ECD)-His to generate a negative control for GDF15/GFRAL/cRET interaction.

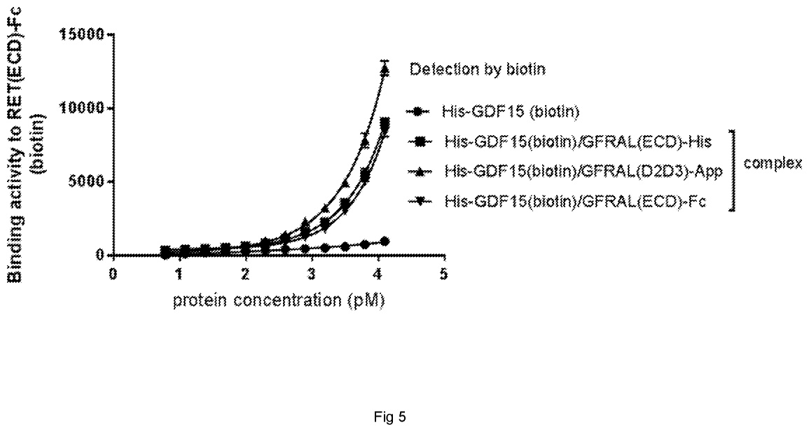

To determine if soluble GFRAL extracellular domains in complex with wild-type GDF15 could bind to immobilized cRET, recombinant human cRET(ECD)-Fc was coated onto meso-scale discovery (MSD) standard bind plates (1 pg protein per ml) in DPBS overnight at 4° C. After washing and blocking, the plates were incubated with 2× serially diluted different GDF15/GFRAL complexes and controls for 60 min and then incubated with streptavidin sulfo-tag (FIG. 5).

Reagents:
Buffer for washing coated plates: DPBS containing 500 μM CaCl₂
Coating buffer: DPBS containing 250 μM CaCl₂
Blocking buffer: DPBS, 5% BSA containing 250 μM CaCl₂)
Dilution buffer: 1× TBST (25 mM Tris, 150 mM NaCl, 0.05% Tween 20) supplemented with 2% BSA, 250 μM CaCl₂
Washing solution: 1× TBST supplemented with 500 μM CaCl₂

Results: All GFRAL/GDF15 complexes freshly made with purified components were capable of binding to cRET(ECD)-Fc coated on plates detected with streptavidin. GFRAL(D2D3)-App complex demonstrated slightly stronger binding activity to GDF15 than full length GFRAL-derived counterparts (GFRAL(ECD)-His and GFRAL(ECD)-Fc) (FIG. 5).

### Example 4: Soluble GFRAL(ECD)-his Alone and Mixed with Mutant GDF15(L294R) do not Bind to Immobilized cRET(ECD)-Fc Protein

Methods: Mixtures of purified biotinylated His-GDF15 and GFRAL(ECD)-His or His-GDF15(L294R) and GFRAL(ECD)-His were prepared as described in Example 3. Individual proteins His-GDF15(L294R) and GFRAL(ECD)-His were also included as controls. Binding of proteins to plates coated with cRET(ECD)-Fc was detected using biotinylated mouse anti-6×His tag monoclonal antibody followed by streptavidin sulfo-tag, and otherwise by methods described in Example 3.

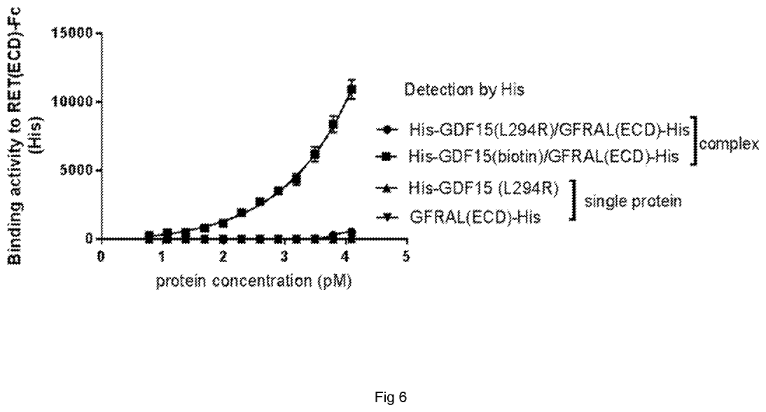

Results: His-GDF15(biotin)/GFRAL(ECD)-His complex demonstrated strong binding to cRET(ECD)-Fc. In contrast, His-GDF15(L294R)/GFRAL(ECD)-His mixture, His-GDF15(L294R) mutant alone, and GFRAL(ECD)-His alone did not show significant binding to cRET(ECD)-Fc (FIG. 6).

### Example 5: Soluble GFRAL(D2D3)-App Combined with his-GDF15 Induces pERK and pAKT in SH-SY5Y Cells

Methods: Co-expressed and co-purified His-GDF15/GFRAL(D2D3)-App and His-GDF15/GFRAL(ECD)-Fc complex were generated as described in Example 2. The reconstituted soluble His-GDF15/GFRAL(D2D3)-App complex was prepared from components purified separately by pre-mixing each component in culture medium and incubation at room temperature for 60 min. The protein complex was then used directly to stimulate SH-SY5Y cells prior to preparation of cell lysates for determination of phosphorylated ERK and AKT protein levels by immunoblot assay. The co-expressed co-purified complexes, the pre-mixed complexes, and the individual proteins were diluted to defined concentration in culture medium, and then used to stimulate SH-SY5Y cells for 15 min prior to detection. Recombinant GDNF protein (Peprotech, Rocky Hill N.J.), which signals through GFRα1 and cRET, was used as a positive control for SH-SY5Y cell activation. The activation of SH-SY5Y cells by the His-GDF15/GFRAL(D2D3)-App complex was detected by immunoblot assay with antibodies against phosphorylated ERK and phosphorylated AKT.

Cell culture and treatment methods: SH-SY5Y cells (American Type Culture Collection (ATCC) CRL-2266) were seeded at 400,000 cells per well in 12-well poly-d-lysine coated plates (Corning; 354470) in DMEM/F12 Ham's media (Life Technologies; 11320-033) containing 10% heat inactivated FBS (Hyclone; SH30071.03) and 1% Penicillin-Streptomycin (Life Technologies; 15140-122). 48 hours later, media was changed to fresh media as described above and additionally containing 1.5 μM retinoic acid. 24 hours later, media was replaced with serum-free DMEM/F12 for two hours. Cells were then treated with proteins or controls for 15 min, washed with warm DPBS, and snap frozen in liquid nitrogen.

Western blot methods: Cells were lysed in RIPA buffer (Life Technologies; 89900) containing protease/phosphatase inhibitor cocktail (Pierce; 78441). Lysates were denatured and reduced and run in NuPAGE 4-12% bis-tris gels (Life Technologies; NP0336BOX) for two hours at 150V. Protein was transferred to nitrocellulose membrane (Life Technologies; 1623001) using the Invitrogen iBlot 2 instrument at 25V for 6 min. Membranes were then blocked in 5% dry milk in tris buffered saline containing 0.1% Tween-20 (TBST) for one hour at room temperature, followed by incubation in primary antibody in TBST containing 5% BSA (Sigma; A8022) overnight at 4° C. Membranes were washed in TBST three times at room temperature for 10 min each, then incubated in secondary antibody for one hour at room temperature in TBST containing 5% BSA. Membranes were then washed three times in TBST for 20 min each at room temperature. Western blots were then visualized using chemiluminescent detection reagent (GE Healthcare; RPN2235; or Perkin Elmer; NEL103001EA).

Antibodies:

Phospho-AKT (Ser473) (Cell Signaling; 4060)—1:2000 dilution

Phospho-p44/42 MAPK (ERK1/2) (Thr202/Tyr204) (Cell Signaling; 4370)—1:2000 dilution

Beta-actin-HRP (Abcam; ab49900)—1:10,000 dilution

Anti-rabbit IgG, HRP-linked (Cell Signaling; 7074)—1:10,000 dilution

Results: Co-expressed and co-purified His-GDF15/GFRAL(D2D3)-App complex and pre-mixed His-GDF15/GFRAL(D2D3)-App was able to induce phosphorylation of ERK and AKT in SH-SY5Y cells in a concentration-dependent manner (FIG. 7, lanes 3-5 and 12). In contrast, co-expressed His-GDF15/GFRAL(ECD)-Fc complex did not stimulate phosphorylation of ERK or AKT in the same cells (FIG. 7, lanes 6-8), despite binding to recombinant cRET(ECD)-Fc immobilized on plates (FIG. 5). Without wishing to be bound by theory, the presence of the Fc on the C-terminus of GFRAL(ECD) may create a steric hindrance which prevents the formation of a functional signaling complex between the GDF15/GFRAL(ECD) and the extracellular region of RET on the cell surface, and thus does not lead to RET dimerization (a prerequisite for RET auto-phosphorylation and signaling). In addition, purified individual components did not stimulate phosphorylation of ERK and AKT in SH-SY5Y cells (FIG. 7, lanes 9-11).

### Example 6: Soluble GFRAL(D2D3) Combined with GDF15 Induces pERK and pAKT in MCF7 Cells

Methods: Co-expressed and reconstituted (pre-mixed) His-GDF15/GFRAL(D2D3)-App complexes were prepared as described in Examples 2 and 5. GDNF protein (which signals through GFRα1 and cRET) was used as a positive control for MCF7 cell activation.

Cell culture and treatment methods: MCF7 cells (ATCC; HTB-22) were seeded at 100,000 cells per well in 12-well tissue culture-treated plates in EMEM media (ATCC; 30-2003) containing 10% heat inactivated FBS (Hyclone; SH30071.03) and 1% Penicillin-Streptomycin (Life Technologies; 15140-122). 48 hours later, media was changed to serum-free EMEM for 24 hours. Cells were then treated with proteins or controls for 15 min, washed with warm DPBS, and snap frozen in liquid nitrogen.

Western blot methods: Western blots were carried out as described in Example 5.

Results: Co-expressed His-GDF15/GFRAL(D2D3)-App complex did not appear to induce phosphorylation of ERK and AKT in MCF7 cells (FIG. 8, lanes 3-5), in contrast to the results in SH-SY5Y cells (FIG. 7, lanes 3-5). However, reconstituted (pre-mixed) His-GDF15/GFRAL(D2D3)-App from individual components was able to induce phosphorylation of ERK and AKT (FIG. 8, lane 12). Similar to results observed with SH-SY5Y cells, co-expressed His-GDF15/GFRAL(ECD)-Fc complex did not induce phosphorylation ERK and AKT in MCF7 cells (FIG. 8, lanes 6-8). Purified individual components also did not stimulate phosphorylation of ERK and AKT in MCF7 cells (FIG. 8, lanes 9-11).

### Example 7: Induction of ERK and AKT Phosphorylation in SH-SY5Y and MCF7 Cells is Dose-Dependent of GDF15/GFRAL(D2D3) Complex

Methods: In Examples 5 and 6, reconstituted (premixed) His-GDF15/GFRAL(D2D3)-App complex was capable of stimulating ERK and AKT phosphorylation in MCF7 and SH-SY5Y cells. To determine if ERK and AKT phosphorylation is dependent on the concentration of reconstituted His-GDF15/GFRAL(D2D3)-App complex, diluted recombinant His-GDF15 (28 nM, 83 nM, and 250 nM) was mixed with equal amounts of GFRAL(D2D3)-App in culture medium. The mixture was incubated at room temperature for 60 min to allow complex formation. The protein complex was then used directly to stimulate MCF7 and SH-SY5Y cells for 15 min prior to preparation of cell lysates for determination of phosphorylated ERK and AKT levels by immunoblot assays, as in Example 5. GDNF protein was used as a positive control for SH-SY5Y cell and MCF7 cell activation.

Cell culture and treatment methods: SH-SY5Y cells were cultured and treated as described in Example 5. MCF7 cells were cultured and treated as described in Example 6.

Western blot methods: Western blots were carried out as described in Example 5.

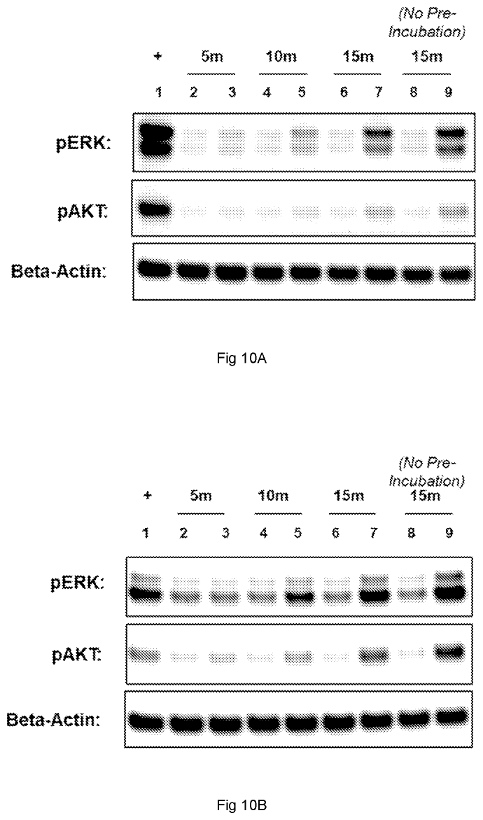

Results: Reconstituted His-GDF15/GFRAL(D2D3)-App complex induction of ERK and AKT phosphorylation in MCF7 and SH-SY5Y cells appears to be dependent on the concentration of the complex (FIG. 9). Stimulated SH-SY5Y cells showed higher total levels of phosphorylated ERK and AKT than stimulated MCF7 cells.

### Example 8: No Extended Pre-Incubation of his-GDF15 and GFRAL(D2D3)-App is Necessary to Reconstitute a Complex that can Activate MCF7 and SH-SY5Y Cells

The following experiments were performed in part to determine the stimulation time needed to induce ERK and AKT phosphorylation in MCF7 and SH-SY5Y cells using the reconstituted His-GDF15/GFRAL(D2D3)-App complex. The experiments were also performed to assess if an extended co-incubation of His-GDF15 and GFRAL(D2D3)-App (prior to addition to MCF7 and SH-SY5Y cells) is necessary to induce ERK and AKT phosphorylation.

Methods: Reconstituted His-GDF15/GFRAL(D2D3)-App complex was prepared as follows. Format (a): 30 nM (for SH-SY5Y cell stimulation) or 100 nM (for MCF7 cell stimulation) of His-GDF15 was mixed with an equal concentration of GFRAL(D2D3)-App in culture medium and incubated at room temperature for 60 min to allow complex formation prior to adding to MCF7 and SH-SY5Y cell cultures. Format (b): the same concentrations of His-GDF15 were mixed with equal concentrations of GFRAL(D2D3)-App in culture medium and then immediately added to MCF7 and SH-SY5Y cell cultures. The results from Format (a) were compared with Format (b) in parallel. GDNF protein was used as a positive control for SH-SY5Y cell and MCF7 cell activation at 3.3 nM.

Cell culture and treatment methods: SH-SY5Y cells were cultured and treated as described in Example 5. MCF7 cells were cultured and treated as described in Example 6.

Western blot methods: Western blots were carried out as described in Example 5.

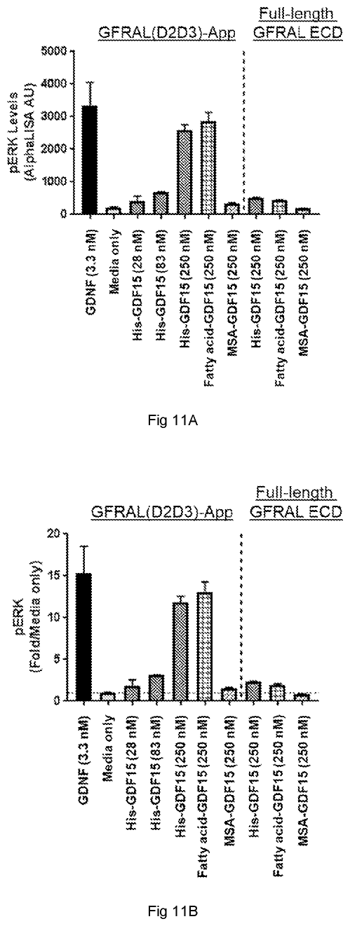

Results: When MCF7 cells were treated with 100 nM complex for 5, 10, and 15 min (FIG. 10A, lanes 3, 5, and 7), the 15 min time point (lane 7) yielded the highest levels of phosphorylated ERK and AKT. The same was true for SH-SY5Y cells treated with 30 nM complex for the same time periods (FIG. 10B, lanes 3, 5, and 7). Stimulation of MCF7 and SH-SY5Y cells for 15 min with reconstituted (pre-mixed) His-GDF15/GFRAL(D2D3)-App that was allowed no pre-incubation time (i.e., added to cells immediately after mixing) also gave rise to high levels of phosphorylated ERK and AKT (FIGS. 10A-B, lane 9). These results suggest that His-GDF15 and GFRAL(D2D3)-App may interact with each other rapidly to form a complex capable of stimulating cells that express RET. No extended co-incubation time is necessary for His-GDF15 and GFRAL(D2D3)-App.

### Example 9: GFRAL(D2D3)-App in Combination with his-GDF15 or Fatty Acid-GDF15 Strongly Stimulates ERK Phosphorylation in MCF7 Cells

The following experiments were performed in part to convert immunoblot-based assays into high throughput plate-based assays (AlphaLISA), and in part to compare the potency of the His-GDF15/GFRAL(D2D3)-App and His-GDF15/GFRAL(ECD)-His complexes.

Methods: Three forms of GDF15 were combined with GFRAL(D2D3)-App or GFRAL(ECD)-His (as described in Example 8), to generate six different GDF15/GFRAL complexes immediately prior to addition of MCF7 cell cultures for induction of ERK phosphorylation. GFD15 samples included His-GDF15, fatty acid-GDF15 (as described in WO 2015/200078), and MSA-GDF15 (a mouse serum albumin-GDF15 fusion, as described in WO 2015/198199 and WO 2017/109706) in DPBS pH 7.4. GDNF protein was used as a positive control for MCF7 cell activation.

Cell culture and treatment methods: MCF7 cells were seeded at 5,000 cells per well in 384-well poly-d-lysine coated plates in EMEM media (ATCC; 30-2003) containing 10% heat inactivated FBS (Hyclone; SH30071.03) and 1% Penicillin-Streptomycin (Life Technologies; 15140-122). 48 hours later, media was changed to serum-free EMEM for 24 hours. Cells were then treated with proteins or controls for 15 min. The cells were then placed on ice for 5 min, and lysis buffer (from kit, Perkin Elmer; ALSU-PERK-A10K) was added to each well. Cells were then shaken at 350 RPM for 10 min at room temperature. Lysates were stored at −80° C. until AlphaLISA was conducted.

AlphaLISA methods: Phospho-ERK levels were detected with the AlphaLISA SureFire Ultra kit (Perkin Elmer, ALSU-PERK-A10K), and the assay was conducted as described by the manufacturer. In brief, 5 μl of diluted acceptor beads were added to each well in 384-well OptiPlate (Perkin Elmer, 6007290); 10 μl of lysate was then added, followed by 5 μl of diluted donor beads; plate was then centrifuged at 1000 RPM for 10 seconds, incubated for 2 hours at room temperature, and then read on an Envision instrument using standard AlphaScreen settings.

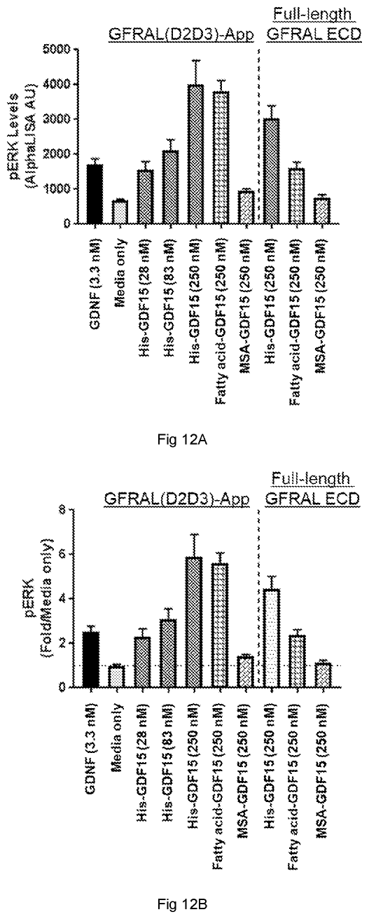

Results: His-GDF15 complexed with GFRAL(D2D3)-App induced ERK phosphorylation in MCF7 cells in a dose-dependent manner (28 nM to 250 nM), as measured by AlphaLISA (FIGS. 11A-B). Fatty acid-GDF15 complexed with GFRAL(D2D3)-App at 250 nM concentration induced similar levels of ERK phosphorylation. The MSA-GDF15 complexed with GFRAL(D2D3)-App at the same concentration induced comparatively little ERK phosphorylation, although levels were slightly above the media only control. This result could be due to the permanent presence of two large MSA polypeptides at the N-termini of the GDF15 dimer, which may create steric hindrances that prevent proper interaction of the GDF15/GFRAL complex with cell surface RET.

Compared to the GFRAL(D2D3)-App protein, the full length GFRAL(ECD)-His protein (when complexed with His-GDF15 or fatty acid-GDF15 at 250 nM concentration) showed low induction of ERK phosphorylation above media control levels. Data shown as both absolute phospho-ERK AlphaLISA assay signal units (FIG. 11A) and fold increase in phosphorylated ERK signal over media control (FIG. 11B).

### Example 10: GFRAL(D2D3)-App in Combination with His-GDF15 or Fatty Acid-GDF15 Strongly Stimulates ERK Phosphorylation in SH-SY5Y Cells

Methods: The following experiment was performed as described in Example 9, with the exception that SH-SY5Y cell cultures were tested.

Cell culture and treatment methods: SH-SY5Y cells were seeded at 10,000 cells per well in 384-well poly-d-lysine coated plates in DMEM/F12 Ham's media (Life Technologies; 11320-033) containing 10% heat inactivated FBS (Hyclone; SH30071.03) and 1% Penicillin-Streptomycin (Life Technologies; 15140-122). 48 hours later, media was changed to fresh media as described above and additionally containing 1.5 μM retinoic acid. 24 hours later, media was replaced with serum-free DMEM/F12 for two hours. Cells were then treated with proteins or controls for 15 min. The cells were then placed on ice for 5 min, and lysis buffer (from kit, Perkin Elmer; ALSU-PERK-A10K) was added to each well. Cell were shaken at 350 RPM for 10 min at room temperature. Lysates were stored at −80° C. until AlphaLISA was conducted.

AlphaLISA methods: Phospho-ERK levels were detected as described in Example 9.

Results: As in MCF7 cells, His-GDF15/GFRAL(D2D3)-App and fatty acid-GDF15/GFRAL(D2D3)-App complexes were more potent than their GFRAL(ECD)-His counterparts in inducing ERK phosphorylation in SH-SY5Y cells (FIGS. 12A-B). However, the activity of the complexes containing GFRAL(ECD)-His appeared greater in the SH-SY5Y cells than in the MCF7 cells.

Additionally, as in MCF7 cells, MSA-GDF15 complexed with GFRAL proteins showed little induction of ERK phosphorylation in SH-SY5Y cells. Data shown as both absolute phospho-ERK AlphaLISA assay signal units (FIG. 12A) and fold increase in phosphorylated ERK signal over media control (FIG. 12B).

### Example 11: ERK Phosphorylation in MCF7 Cells is Dependent on the Dose of GFRAL(D2D3) and Fatty Acid-GDF15

Methods: In these experiments, fatty acid-GDF15 was combined with GFRAL(D2D3)-App at various concentrations to compare the relative ability of each to induce ERK phosphorylation in MCF7 cells. Experimental methods were performed as described in Example 9.

Cell culture and treatment methods: MCF7 cells were cultured and treated as described in Example 9.

AlphaLISA methods: Phospho-ERK levels were detected as described in Example 9.

Results: Complexes containing higher concentrations of both fatty acid-GDF15 and GFRAL(D2D3)-App led to larger inductions of ERK phosphorylation above the media control (Table 3).

There is a ratio of the two that achieve maximal pERK signal and increasing fatty acid-GDF15 concentrations to or beyond the concentrations of GFRAL(D2D3)-App may lead to a reduction of signal from the peak value. This hypothesis is in agreement with a ternary complex model. As fatty acid-GDF15 concentrations match or exceed those of GFRAL(D2D3), there may be increased formation of fatty acid-GDF15 complexes with a single GFRAL(D2D3)-App protein. Such complexes would not be expected to bind two RET proteins, in contrast to fatty acid-GDF15 complexes with two GFRAL(D2D3)-App proteins, which are capable of binding two RET proteins that dimerize and actively signal. Thus, ideally the concentration of the GDF15 construct in the assay does not exceed the concentration of the GFRAL(D2D3) construct, in order for the assay to accurately compare the potency of different GDF15-based materials.

The dose-dependent results of fatty acid-GDF15 combined with GFRAL(D2D3)-App on ERK phosphorylation in MCF7 cells are reproducible (Table 4).

TABLE 3
ERK phosphorylation in MCF7 cells following treatment
with GFRAL(D2D3)-App and fatty acid-GDF15*

| Fatty acid- | GFRAL(D2D3)-App (nM) | | | | | | | |
|---|---|---|---|---|---|---|---|---|
| GDF15 (nM) | 0 | 31.25 | 62.5 | 125 | 250 | 500 | 1000 | 2000 |
| 0 | 1.00 | 1.09 | 1.16 | 0.83 | 1.07 | 1.48 | 3.21 | 4.20 |
| 31.25 | 0.81 | 2.12 | 3.52 | 4.70 | 3.46 | 5.24 | 9.51 | 11.54 |
| 62.5 | 0.93 | 2.11 | 4.65 | 10.39 | 9.63 | 12.42 | 16.79 | 13.57 |
| 125 | 0.83 | 1.46 | 4.03 | 8.63 | 13.76 | 21.01 | 21.59 | 19.35 |
| 250 | 0.84 | 1.06 | 1.82 | 5.33 | 15.53 | 21.17 | 28.96 | 33.82 |
| 500 | 0.83 | 0.97 | 1.37 | 2.95 | 9.51 | 17.57 | 31.31 | 30.09 |
| 1000 | 0.81 | 0.85 | 1.03 | 1.96 | 10.19 | 27.64 | 24.62 | 30.09 |
| 2000 | 0.79 | 0.73 | 0.98 | 1.30 | 3.55 | 16.97 | 23.17 | 20.32 |

*Values shown in chart indicate pERK fold change.

TABLE 4
ERK phosphorylation in MCF7 cells following treatment
with GFRAL(D2D3)-App and fatty acid-GDF15*

| | GFRAL(D2D3)-APP (nM) | | | | | |
|---|---|---|---|---|---|---|
| Fatty acid-GDF15 (nM) | 0 | 31.25 | 62.5 | 125 | 250 | 500 |
| 0 | 1.00 | 1.16 | 1.08 | 1.07 | 1.36 | 2.31 |
| 31.25 | 0.96 | 2.89 | 5.35 | 6.30 | 7.17 | 7.26 |
| 62.5 | 0.81 | 2.94 | 8.99 | 8.96 | 10.77 | 11.06 |
| 125 | 0.87 | 2.49 | 7.48 | 8.19 | 14.10 | 15.70 |
| 250 | 0.74 | 1.62 | 5.08 | 12.74 | 23.29 | 25.22 |
| 500 | 0.80 | 0.88 | 3.67 | 7.84 | 19.00 | 28.68 |
| 1000 | 0.74 | 0.88 | 1.48 | 4.06 | 11.35 | 22.17 |
| 2000 | 0.91 | 0.84 | 1.11 | 2.79 | 8.52 | 18.77 |

*Values shown in chart indicate pERK fold change.

### NUMBERED EMBODIMENTS

Embodiment 1. A method of detecting the activity of a GDF15 peptide, comprising:
(a) providing a cell that expresses a cell surface receptor kinase;
(b) contacting the cell with the GDF15 peptide and a soluble GFRAL; and
(c) detecting a biological response in the contacted cell, wherein the soluble GFRAL comprises a GFRAL extracellular domain comprising domains D2 and D3.
Embodiment 2. The method of Embodiment 1, wherein the soluble GFRAL comprises a GFRAL extracellular domain lacking domain D1.
Embodiment 3. The method of embodiment 1 or 2, wherein the soluble GFRAL further comprises a signal peptide.
Embodiment 4. The method of any one of embodiments 1 to 3, wherein the soluble GFRAL comprises the amino acid sequence of SEQ ID NO:1 or a functional variant thereof.
Embodiment 5. The method of any one of embodiments 1 to 3, wherein the soluble GFRAL or functional variant has at least 80% amino acid sequence identity with the amino acid sequence of SEQ ID NO:1.
Embodiment 6. The method of any one of embodiments 1 to 3, wherein the soluble GFRAL or functional variant has at least 90% amino acid sequence identity with the amino acid sequence of SEQ ID NO:1.
Embodiment 7. The method of any one of embodiments 1 to 3, wherein the soluble GFRAL comprises the amino acid sequence of SEQ ID NO:2 or a functional variant thereof.
Embodiment 8. The method of any one of embodiments 1 to 7, wherein the soluble GFRAL further comprises (e.g., is fused to) an affinity tag.
Embodiment 9. The method of embodiment 8, wherein the affinity tag comprises an amyloid-beta precursor protein tag, a histidine tag, a FLAG tag, or a myc tag.
Embodiment 10. The method of any one of embodiments 1 to 9, wherein the GDF15 peptide comprises the amino acid sequence of SEQ ID NO:13 or a functional variant thereof.
Embodiment 11. The method of any one of embodiments 1 to 9, wherein the GDF15 peptide or functional variant has at least 80% amino acid sequence identity with the amino acid sequence of SEQ ID NO:13.
Embodiment 12. The method of any one of embodiments 1 to 9, wherein the GDF15 peptide or functional variant has at least 90% amino acid sequence identity with the amino acid sequence of SEQ ID NO:13.
Embodiment 13. The method of any one of embodiments 1 to 12, wherein the GDF15 peptide comprises an affinity tag, a fusion, a conjugation, a PEGylation, and/or a glycosylation.
Embodiment 14. The method of any one of embodiments 1 to 13, wherein the GDF15 peptide is tagged with an amyloid-beta precursor protein tag, a histidine tag, a FLAG tag, or a myc tag.
Embodiment 15. The method of any one of embodiments 1 to 14, wherein the GDF15 peptide is fused to a human serum albumin, a mouse serum albumin, an immunoglobulin constant region, or an alpha-1-antitrypsin.
Embodiment 16. The method of any one of embodiments 1 to 15, wherein the GDF15 peptide is conjugated to a fatty acid.
Embodiment 17. The method of any one of embodiments 1 to 16, wherein the cell is contacted with the GDF15 peptide and the soluble GFRAL simultaneously.
Embodiment 18. The method of any one of embodiments 1 to 16, wherein the cell is contacted with the GDF15 peptide and the soluble GFRAL sequentially.
Embodiment 19. The method of embodiment 17, wherein the GDF15 peptide and the soluble GFRAL are in the same composition.
Embodiment 20. The method of embodiment 19, wherein the GDF15 peptide and the soluble GFRAL are in a mixture.
Embodiment 21. The method of embodiment 19, wherein the GDF15 peptide and the soluble GFRAL are in a binary complex.
Embodiment 22. The method of any one of embodiments 1 to 21, wherein the cell surface receptor kinase is an endogenous cell surface receptor kinase.
Embodiment 23. The method of any one of embodiments 1 to 21, wherein the cell surface receptor kinase is an exogenous cell surface receptor kinase.
Embodiment 24. The method of any one of embodiments 1 to 23, wherein the cell surface receptor kinase is a RET receptor tyrosine kinase.
Embodiment 25. The method of any one of embodiments 1 to 24, wherein the cell does not express endogenous GFRAL.
Embodiment 26. The method of any one of embodiments 1 to 25, wherein the cell does not express full length GFRAL.
Embodiment 27. The method of any one of embodiments 1 to 26, wherein the cell does not express endogenous GDF15.
Embodiment 28. The method of any one of embodiments 1 to 27, wherein the cell is a GDF15 knockout (KO) cell comprising an inoperative GDF15 gene.
Embodiment 29. The method of any one of embodiments 1 to 28, wherein the cell is a mammalian cell.
Embodiment 30. The method of any one of embodiments 1 to 29, wherein the cell is a human cell.
Embodiment 31. The method of any one of embodiments 1 to 30, wherein the cell is an MCF7 cell.
Embodiment 32. The method of any one of embodiments 1 to 30, wherein the cell is an SH-SY5Y cell.
Embodiment 33. The method of any one of embodiments 1 to 30, wherein the cell is an HEK293A-GDF15 KO cell.
Embodiment 34. The method of any one of embodiments 1 to 33, wherein the biological response is induced when the GDF15 peptide, the soluble GFRAL, and the cell surface receptor kinase form a ternary complex.
Embodiment 35. The method of any one of embodiments 1 to 34, wherein the biological response is not induced in a cell contacted with the GDF15 peptide in the absence of the soluble GFRAL.
Embodiment 36. The method of any one of embodiments 1 to 35, wherein the biological response is an increase or decrease in the expression or activity of a protein in the cell, as compared to the expression or activity of the same protein in a control cell that is not contacted with the GDF15 peptide and the soluble GFRAL.
Embodiment 37. The method of any one of embodiments 1 to 36, wherein the biological response is an increase or decrease in the expression or activity of an intracellular protein in one or more of the RET-ERK, RET-AKT, protein kinase C, JAK/STAT, JNK, p38, and RAC1 pathways.
Embodiment 38. The method of embodiment 36, wherein the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 39. The method of embodiment 36, wherein the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 40. The method of embodiment 36 or embodiment 38, wherein the cell surface receptor kinase is a RET receptor tyrosine kinase and the protein is an intracellular protein in the RET-ERK pathway.
Embodiment 41. The method of embodiment 40, wherein the intracellular protein is selected from one or more of ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2, or any downstream targets thereof.
Embodiment 42. The method of embodiment 40 or embodiment 41, wherein the intracellular protein is selected from one or more of ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 43. The method of embodiment 41 or embodiment 42, wherein the ERK is ERK1 or ERK2.
Embodiment 44. The method of embodiment 36 or embodiment 39, wherein the cell surface receptor kinase is a RET receptor tyrosine kinase and the protein is an intracellular protein in the RET-AKT pathway.
Embodiment 45. The method of embodiment 44, wherein the intracellular protein is selected from one or more of AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR, or any downstream targets thereof.
Embodiment 46. The method of embodiment 44 or embodiment 45, wherein the intracellular protein is selected from one or more of AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 47. The method of embodiment 45 or embodiment 46, wherein the AKT is AKT1, AKT2, or AKT3.
Embodiment 48. The method of any one of embodiments 1 to 35, wherein the biological response is an increase or decrease in phosphorylation of a protein kinase in the cell, as compared to phosphorylation of the same protein kinase in a control cell that is not contacted with the GDF15 peptide and the soluble GFRAL.
Embodiment 49. The method of embodiment 48, wherein the protein kinase is the cell surface receptor kinase.
Embodiment 50. The method of embodiment 48 or embodiment 49, wherein the protein kinase and/or cell surface receptor kinase is a RET receptor tyrosine kinase.
Embodiment 51. The method of embodiment 48, wherein the protein kinase is an intracellular protein kinase, wherein the intracellular protein kinase is directly or indirectly phosphorylated by the cell surface receptor kinase.
Embodiment 52. The method of embodiment 48 or embodiment 51, wherein the protein kinase is an intracellular protein kinase in the RET-ERK pathway.
Embodiment 53. The method of embodiment 52, wherein the intracellular protein kinase is selected from one or more of ERK, JAK1, JAK2, RAF, MEK1, MEK2, RSK1, RSK2, RSK3, MNK1, MNK2, MSK1, and MSK2, or any downstream targets thereof.
Embodiment 54. The method of embodiment 52 or embodiment 53, wherein the intracellular protein kinase is selected from one or more of ERK, JAK1, JAK2, RAF, MEK1, MEK2, RSK1, RSK2, RSK3, MNK1, MNK2, MSK1, and MSK2.
Embodiment 55. The method of any one of embodiments 52 to 54, wherein the intracellular protein kinase is ERK.
Embodiment 56. The method of any one of embodiments 53 to 55, wherein the ERK is ERK1 or ERK2.
Embodiment 57. The method of embodiment 48 or embodiment 51, wherein the protein kinase is an intracellular protein kinase in the RET-AKT pathway.
Embodiment 58. The method of embodiment 57, wherein the intracellular protein kinase is selected from one or more of AKT, SRC, JAK1, JAK2, PI3K, PDK1, MLK3, ASK1, GSK3alpha, GSK3beta, and mTOR, or any downstream targets thereof.
Embodiment 59. The method of embodiment 57 or embodiment 58, wherein the intracellular protein kinase is selected from one or more of AKT, SRC, JAK1, JAK2, PI3K, PDK1, MLK3, ASK1, GSK3alpha, GSK3beta, and mTOR.
Embodiment 60. The method of any one of embodiments 57 to 59, wherein the intracellular protein kinase is AKT.
Embodiment 61. The method of any one of embodiments 58 to 60, wherein the AKT is AKT1, AKT2, or AKT3.
Embodiment 62. A method of detecting the activity of a GDF15 peptide, comprising:
(a) providing a cell that expresses a GFRAL extracellular domain and a cell surface receptor kinase;
(b) contacting the cell with the GDF15 peptide; and
(c) detecting a biological response in the contacted cell, wherein the GFRAL extracellular domain comprises domains D2 and D3.
Embodiment 63. The method of embodiment 62, wherein the GFRAL extracellular domain lacks domain D1.
Embodiment 64. The method of embodiment 62 or embodiment 63, wherein the GFRAL extracellular domain is a soluble GFRAL extracellular domain.
Embodiment 65. The method of embodiment 62 or embodiment 63, wherein the GFRAL extracellular domain is attached to the cell surface by a tether.
Embodiment 66. The method of embodiment 65, wherein the tether is a GFRAL transmembrane domain or a functional fragment thereof.
Embodiment 67. The method of embodiment 65 or embodiment 66, wherein the GFRAL extracellular domain or tether comprises the amino acid sequence of SEQ ID NO:18 or a functional variant thereof.
Embodiment 68. The method of embodiment 65, wherein the tether is a heterologous transmembrane domain fused to the GFRAL extracellular domain.
Embodiment 69. The method of embodiment 65, wherein the tether is a glycophosphatidylinositol (GPI).
Embodiment 70. The method of embodiment 65 or embodiment 69, wherein the GFRAL extracellular domain or tether comprises the amino acid sequence of SEQ ID NO:19 or a functional variant thereof, SEQ ID NO:20 or a functional variant thereof, or SEQ ID NO:21 or a functional variant thereof.
Embodiment 71. The method of embodiment 65, wherein the tether is a membrane-inserting sequence.
Embodiment 72. The method of embodiment 65 or embodiment 71, wherein the GFRAL extracellular domain or tether comprises the amino acid sequence of SEQ ID NO:22 or a functional variant thereof, or SEQ ID NO:23 or a functional variant thereof.
Embodiment 73. The method of embodiment 65, wherein the tether is a membrane-inserting fatty acid.
Embodiment 74. The method of any one of embodiments 62 to 73, wherein the GFRAL extracellular domain further comprises a signal peptide.
Embodiment 75. The method of any one of embodiments 62 to 74, wherein the GFRAL extracellular domain comprises the amino acid sequence of SEQ ID NO:1 or a functional variant thereof.
Embodiment 76. The method of any one of embodiments 62 to 74, wherein the GFRAL extracellular domain or functional variant has at least 80% amino acid sequence identity with the amino acid sequence of SEQ ID NO:1.
Embodiment 77. The method of any one of embodiments 62 to 74, wherein the GFRAL extracellular domain or functional variant has at least 90% amino acid sequence identity with the amino acid sequence of SEQ ID NO:1.
Embodiment 78. The method of any one of embodiments 62 to 74, wherein the GFRAL extracellular domain comprises the amino acid sequence of SEQ ID NO:2 or a functional variant thereof.
Embodiment 79. The method of any one of embodiments 62 to 78, wherein the GDF15 peptide comprises the amino acid sequence of SEQ ID NO:13 or a functional variant thereof.
Embodiment 80. The method of any one of embodiments 62 to 78, wherein the GDF15 peptide or functional variant has at least 80% amino acid sequence identity with the amino acid sequence of SEQ ID NO:13.
Embodiment 81. The method of any one of embodiments 62 to 78, wherein the GDF15 peptide or functional variant has at least 90% amino acid sequence identity with the amino acid sequence of SEQ ID NO:13.
Embodiment 82. The method of any one of embodiments 62 to 81, wherein the GDF15 peptide comprises an affinity tag, a fusion, a conjugation, a PEGylation, and/or a glycosylation.
Embodiment 83. The method of any one of embodiments 62 to 82, wherein the GDF15 peptide is tagged with an amyloid-beta precursor protein tag, a histidine tag, a FLAG tag, or a myc tag.
Embodiment 84. The method of any one of embodiments 62 to 83, wherein the GDF15 peptide is fused to a human serum albumin, a mouse serum albumin, an immunoglobulin constant region, or an alpha-1-antitrypsin.
Embodiment 85. The method of any one of embodiments 62 to 84, wherein the GDF15 peptide is conjugated to a fatty acid.
Embodiment 86. The method of any one of embodiments 62 to 85, wherein the cell surface receptor kinase is an endogenous cell surface receptor kinase.
Embodiment 87. The method of any one of embodiments 62 to 85, wherein the cell surface receptor kinase is an exogenous cell surface receptor kinase.
Embodiment 88. The method of any one of embodiments 62 to 87, wherein the cell surface receptor kinase is a RET receptor tyrosine kinase.
Embodiment 89. The method of any one of embodiments 62 to 88, wherein the cell does not express endogenous GFRAL.
Embodiment 90. The method of any one of embodiments 62 to 89, wherein the cell does not express full length GFRAL.
Embodiment 91. The method of any one of embodiments 62 to 90, wherein the cell does not express endogenous GDF15.
Embodiment 92. The method of any one of embodiments 62 to 91, wherein the cell is a GDF15 KO cell comprising an inoperative GDF15 gene.
Embodiment 93. The method of any one of embodiments 62 to 92, wherein the cell is a mammalian cell.
Embodiment 94. The method of any one of embodiments 62 to 93, wherein the cell is a human cell.
Embodiment 95. The method of any one of embodiments 62 to 94, wherein the cell is an MCF7 cell.
Embodiment 96. The method of any one of embodiments 62 to 94, wherein the cell is an SH-SY5Y cell.
Embodiment 97. The method of any one of embodiments 62 to 94, wherein the cell is an HEK293A-GDF15 KO cell.
Embodiment 98. The method of any one of embodiments 62 to 97, wherein the biological response is induced when the GDF15 peptide, the GFRAL extracellular domain, and the cell surface receptor kinase form a ternary complex.
Embodiment 99. The method of any one of embodiments 62 to 98, wherein the biological response is an increase or decrease in the expression or activity of a protein in the cell, as compared to the expression or activity of the same protein in a control cell that is not contacted with the GDF15 peptide.
Embodiment 100. The method of any one of embodiments 62 to 99, wherein the biological response is an increase or decrease in the expression or activity of an intracellular protein in one or more of the RET-ERK, RET-AKT, protein kinase C, JAK/STAT, JNK, p38, and RAC1 pathways.
Embodiment 101. The method of embodiment 99, wherein the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 102. The method of embodiment 99, wherein the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 103. The method of embodiment 99 or embodiment 101, wherein the cell surface receptor kinase is a RET receptor tyrosine kinase and the protein is an intracellular protein in the RET-ERK pathway.
Embodiment 104. The method of embodiment 103, wherein the intracellular protein is selected from one or more of ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2, or any downstream targets thereof.
Embodiment 105. The method of embodiment 103 or embodiment 104, wherein the intracellular protein is selected from one or more of ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 106. The method of embodiment 104 or embodiment 105, wherein the ERK is ERK1 or ERK2.
Embodiment 107. The method of embodiment 99 or embodiment 102, wherein the cell surface receptor kinase is a RET receptor tyrosine kinase and the protein is an intracellular protein in the RET-AKT pathway.
Embodiment 108. The method of embodiment 107, wherein the intracellular protein is selected from one or more of AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR, or any downstream targets thereof.
Embodiment 109. The method of embodiment 107 or embodiment 108, wherein the intracellular protein is selected from one or more of AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 110. The method of embodiment 108 or embodiment 109, wherein the AKT is AKT1, AKT2, or AKT3.
Embodiment 111. The method of any one of embodiments 62 to 98, wherein the biological response is an increase or decrease in phosphorylation of a protein kinase in the cell, as compared to phosphorylation of the same protein kinase in a control cell that is not contacted with the GDF15 peptide.
Embodiment 112. The method of embodiment 111, wherein the protein kinase is the cell surface receptor kinase.
Embodiment 113. The method of embodiment 111 or embodiment 112, wherein the protein kinase and/or cell surface receptor kinase is a RET receptor tyrosine kinase.
Embodiment 114. The method of embodiment 111, wherein the protein kinase is an intracellular protein kinase, wherein the intracellular protein kinase is directly or indirectly phosphorylated by the cell surface receptor kinase.
Embodiment 115. The method of embodiment 111 or embodiment 114, wherein the protein kinase is an intracellular protein kinase in the RET-ERK pathway.
Embodiment 116. The method of embodiment 115, wherein the intracellular protein kinase is selected from one or more of ERK, JAK1, JAK2, RAF, MEK1, MEK2, RSK1, RSK2, RSK3, MNK1, MNK2, MSK1, and MSK2, or any downstream targets thereof.
Embodiment 117. The method of embodiment 115 or embodiment 116, wherein the intracellular protein kinase is selected from one or more of ERK, JAK1, JAK2, RAF, MEK1, MEK2, RSK1, RSK2, RSK3, MNK1, MNK2, MSK1, and MSK2.
Embodiment 118. The method of any one of embodiments 115 to 117, wherein the intracellular protein kinase is ERK.
Embodiment 119. The method of any one of embodiments 116 to 118, wherein the ERK is ERK1 or ERK2.
Embodiment 120. The method of embodiment 111 or embodiment 114, wherein the protein kinase is an intracellular protein kinase in the RET-AKT pathway.
Embodiment 121. The method of embodiment 120, wherein the intracellular protein kinase is selected from one or more of AKT, SRC, JAK1, JAK2, PI3K, PDK1, MLK3, ASK1, GSK3alpha, GSK3beta, and mTOR, or any downstream targets thereof.
Embodiment 122. The method of embodiment 120 or embodiment 121, wherein the intracellular protein kinase is selected from one or more of AKT, SRC, JAK1, JAK2, PI3K, PDK1, MLK3, ASK1, GSK3alpha, GSK3beta, and mTOR.
Embodiment 123. The method of any one of embodiments 120 to 122, wherein the intracellular protein kinase is AKT.
Embodiment 124. The method of any one of embodiments 121 to 123, wherein the AKT is AKT1, AKT2, or AKT3.
Embodiment 125. An isolated and modified cell for detecting the activity of a GDF15 peptide, wherein the cell expresses a GFRAL extracellular domain comprising domains D2 and D3 and a cell surface receptor kinase.
Embodiment 126. The cell of embodiment 125, wherein the GFRAL extracellular domain lacks domain D1.
Embodiment 127. The cell of embodiment 125 or embodiment 126, wherein the GFRAL extracellular domain is a soluble GFRAL extracellular domain.
Embodiment 128. The cell of embodiment 125 or embodiment 126, wherein the GFRAL extracellular domain is attached to the cell surface by a tether.
Embodiment 129. The cell of embodiment 128, wherein the tether is a GFRAL transmembrane domain or a functional fragment thereof.
Embodiment 130. The cell of embodiment 128 or embodiment 129, wherein the GFRAL extracellular domain or tether comprises the amino acid sequence of SEQ ID NO:18 or a functional variant thereof.
Embodiment 131. The cell of embodiment 128, wherein the tether is a heterologous transmembrane domain fused to the GFRAL extracellular domain.
Embodiment 132. The cell of embodiment 128, wherein the tether is a glycophosphatidylinositol (GPI).
Embodiment 133. The cell of embodiment 128 or embodiment 132, wherein the GFRAL extracellular domain or tether comprises the amino acid sequence of SEQ ID NO:19 or a functional variant thereof, SEQ ID NO:20 or a functional variant thereof, or SEQ ID NO:21 or a functional variant thereof.
Embodiment 134. The cell of embodiment 128, wherein the tether is a membrane-inserting sequence.
Embodiment 135. The cell of embodiment 128 or embodiment 134, wherein the GFRAL extracellular domain or tether comprises the amino acid sequence of SEQ ID NO:22 or a functional variant thereof, or SEQ ID NO:23 or a functional variant thereof.
Embodiment 136. The cell of embodiment 128, wherein the tether is a membrane-inserting fatty acid.
Embodiment 137. The cell of any one of embodiments 125 to 136, wherein the GFRAL extracellular domain further comprises a signal peptide.
Embodiment 138. The cell of any one of embodiments 125 to 137, wherein the GFRAL extracellular domain comprises the amino acid sequence of SEQ ID NO:1 or a functional variant thereof.
Embodiment 139. The cell of any one of embodiments 125 to 137, wherein the GFRAL extracellular domain or functional variant has at least 80% amino acid sequence identity with the amino acid sequence of SEQ ID NO:1.
Embodiment 140. The cell of any one of embodiments 125 to 137, wherein the GFRAL extracellular domain or functional variant has at least 90% amino acid sequence identity with the amino acid sequence of SEQ ID NO:1.
Embodiment 141. The cell of any one of embodiments 125 to 137, wherein the GFRAL extracellular domain comprises the amino acid sequence of SEQ ID NO:2 or a functional variant thereof.
Embodiment 142. The cell of any one of embodiments 125 to 141, wherein the GDF15 peptide comprises the amino acid sequence of SEQ ID NO:13 or a functional variant thereof.
Embodiment 143. The cell of any one of embodiments 125 to 141, wherein the GDF15 peptide or functional variant has at least 80% amino acid sequence identity with the amino acid sequence of SEQ ID NO:13.
Embodiment 144. The cell of any one of embodiments 125 to 141, wherein the GDF15 peptide or functional variant has at least 90% amino acid sequence identity with the amino acid sequence of SEQ ID NO:13.
Embodiment 145. The cell of any one of embodiments 125 to 144, wherein the GDF15 peptide comprises an affinity tag, a fusion, a conjugation, a PEGylation, and/or a glycosylation.
Embodiment 146. The cell of any one of embodiments 125 to 145, wherein the GDF15 peptide is tagged with an amyloid-beta precursor protein tag, a histidine tag, a FLAG tag, or a myc tag.
Embodiment 147. The cell of any one of embodiments 125 to 146, wherein the GDF15 peptide is fused to a human serum albumin, a mouse serum albumin, an immunoglobulin constant region, or an alpha-1-antitrypsin.
Embodiment 148. The cell of any one of embodiments 125 to 147, wherein the GDF15 peptide is conjugated to a fatty acid.
Embodiment 149. The cell of any one of embodiments 125 to 148, wherein the cell surface receptor kinase is an endogenous cell surface receptor kinase.
Embodiment 150. The cell of any one of embodiments 125 to 148, wherein the cell surface receptor kinase is an exogenous cell surface receptor kinase.
Embodiment 151. The cell of any one of embodiments 125 to 150, wherein the cell surface receptor kinase is a RET receptor tyrosine kinase.
Embodiment 152. The cell of any one of embodiments 125 to 151, wherein the cell does not express endogenous GFRAL.
Embodiment 153. The cell of any one of embodiments 125 to 152, wherein the cell does not express full length GFRAL.
Embodiment 154. The cell of any one of embodiments 125 to 153, wherein the cell does not express endogenous GDF15.
Embodiment 155. The cell of any one of embodiments 125 to 154, wherein the cell is a GDF15 KO cell comprising an inoperative GDF15 gene.
Embodiment 156. The cell of any one of embodiments 125 to 155, wherein the cell is a mammalian cell.
Embodiment 157. The cell of any one of embodiments 125 to 156, wherein the cell is a human cell.
Embodiment 158. The cell of any one of embodiments 125 to 157, wherein the cell is an MCF7 cell.
Embodiment 159. The cell of any one of embodiments 125 to 157, wherein the cell is an SH-SY5Y cell.
Embodiment 160. The cell of any one of embodiments 125 to 157, wherein the cell is an HEK293A-GDF15 KO cell.
Embodiment 161. A kit for determining the activity of a GDF15 peptide, wherein the kit comprises the cell of any one of embodiments 125 to 160 for contacting with the GDF15 peptide; and a means of detecting a biological response in the contacted cell.
Embodiment 162. A method of treating obesity or an obesity-related disorder, comprising administering a GDF15 peptide to a subject, wherein the GDF15 peptide induces a biological response in a cell contacted with the GDF15 peptide, wherein the biological response is or can be detected by the method of any one of embodiments 1 to 124.
Embodiment 163. The method of embodiment 162, wherein the GDF15 peptide comprises the amino acid sequence of SEQ ID NO:13 or a functional variant thereof.
Embodiment 164. The method of embodiment 162, wherein the GDF15 peptide or functional variant has at least 80% amino acid sequence identity with the amino acid sequence of SEQ ID NO:13.
Embodiment 165. The method of embodiment 162, wherein the GDF15 peptide or functional variant has at least 90% amino acid sequence identity with the amino acid sequence of SEQ ID NO:13.
Embodiment 166. The method of any one of embodiments 162 to 165, wherein the GDF15 peptide comprises an affinity tag, a fusion, a conjugation, a PEGylation, and/or a glycosylation.
Embodiment 167. The method of any one of embodiments 162 to 166, wherein the GDF15 peptide is tagged with an amyloid-beta precursor protein tag, a histidine tag, a FLAG tag, or a myc tag.
Embodiment 168. The method of any one of embodiments 162 to 167, wherein the GDF15 peptide is fused to a human serum albumin, a mouse serum albumin, an immunoglobulin constant region, or an alpha-1-antitrypsin.
Embodiment 169. The method of any one of embodiments 162 to 168, wherein the GDF15 peptide is conjugated to a fatty acid.
Embodiment 170. The method of any one of embodiments 162 to 169, wherein the biological response is a signal transduction response.
Embodiment 171. The method of any one of embodiments 162 to 170, wherein the biological response is an increase or decrease in the expression or activity of a protein in the cell, as compared to the expression or activity of the same protein in a control cell that is not contacted with the GDF15 peptide.
Embodiment 172. The method of any one of embodiments 162 to 171, wherein the biological response is an increase or decrease in the expression or activity of an intracellular protein in one or more of the RET-ERK, RET-AKT, protein kinase C, JAK/STAT, JNK, p38, and RAC1 pathways.
Embodiment 173. The method of embodiment 171, wherein the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 174. The method of embodiment 171, wherein the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 175. The method of any one of embodiments 162 to 170, wherein the biological response is an increase or decrease in phosphorylation of a protein kinase in the cell, as compared to phosphorylation of the same protein kinase in a control cell that is not contacted with the GDF15 peptide.
Embodiment 176. The method of embodiment 175, wherein the protein kinase is an intracellular protein kinase, wherein the intracellular protein kinase is directly or indirectly phosphorylated by the cell surface receptor kinase.
Embodiment 177. The method of any one of embodiments 162 to 176, wherein the subject is overweight or obese.
Embodiment 178. The method of any one of embodiments 162 to 177, wherein the subject has a body mass index between 25 and 29.9.
Embodiment 179. The method of any one of embodiments 162 to 177, wherein the subject has a body mass index of 30 or higher.
Embodiment 180. The method of any one of embodiments 162 to 179, wherein the obesity-related disorder is a cancer, a body weight disorder, or a metabolic disease or disorder.
Embodiment 181. The method of any one of embodiments 162 to 180, wherein the obesity-related disorder is a cancer, type II diabetes mellitus (T2DM), nonalcoholic steatohepatitis (NASH), hypertriglyceridemia, or cardiovascular disease.
Embodiment 182. Use of a GDF15 peptide in treating obesity or an obesity-related disorder in a subject, wherein the GDF15 peptide induces a biological response in a cell contacted with the GDF15 peptide, wherein the biological response is or can be detected by the method of any one of embodiments 1 to 124.
Embodiment 183. The use of embodiment 182, wherein the GDF15 peptide comprises the amino acid sequence of SEQ ID NO:13 or a functional variant thereof.
Embodiment 184. The use of embodiment 182, wherein the GDF15 peptide or functional variant has at least 80% amino acid sequence identity with the amino acid sequence of SEQ ID NO:13.
Embodiment 185. The use of embodiment 182, wherein the GDF15 peptide or functional variant has at least 90% amino acid sequence identity with the amino acid sequence of SEQ ID NO:13.
Embodiment 186. The use of any one of embodiments 182 to 185, wherein the GDF15 peptide comprises an affinity tag, a fusion, a conjugation, a PEGylation, and/or a glycosylation.
Embodiment 187. The use of any one of embodiments 182 to 186, wherein the GDF15 peptide is tagged with an amyloid-beta precursor protein tag, a histidine tag, a FLAG tag, or a myc tag.
Embodiment 188. The use of any one of embodiments 182 to 187, wherein the GDF15 peptide is fused to a human serum albumin, a mouse serum albumin, an immunoglobulin constant region, or an alpha-1-antitrypsin.
Embodiment 189. The use of any one of embodiments 182 to 188, wherein the GDF15 peptide is conjugated to a fatty acid.
Embodiment 190. The use of any one of embodiments 182 to 189, wherein the biological response is a signal transduction response.
Embodiment 191. The use of any one of embodiments 182 to 190, wherein the biological response is an increase or decrease in the expression or activity of a protein in the cell, as compared to the expression or activity of the same protein in a control cell that is not contacted with the GDF15 peptide.
Embodiment 192. The use of any one of embodiments 182 to 191, wherein the biological response is an increase or decrease in the expression or activity of an intracellular protein in one or more of the RET-ERK, RET-AKT, protein kinase C, JAK/STAT, JNK, p38, and RAC1 pathways.
Embodiment 193. The use of embodiment 191, wherein the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 194. The use of embodiment 191, wherein the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 195. The use of any one of embodiments 182 to 190, wherein the biological response is an increase or decrease in phosphorylation of a protein kinase in the cell, as compared to phosphorylation of the same protein kinase in a control cell that is not contacted with the GDF15 peptide.
Embodiment 196. The use of embodiment 195, wherein the protein kinase is an intracellular protein kinase, wherein the intracellular protein kinase is directly or indirectly phosphorylated by the cell surface receptor kinase.
Embodiment 197. The use of any one of embodiments 182 to 196, wherein the subject is overweight or obese.
Embodiment 198. The use of any one of embodiments 182 to 197, wherein the subject has a body mass index between 25 and 29.9.
Embodiment 199. The use of any one of embodiments 182 to 197, wherein the subject has a body mass index of 30 or higher.
Embodiment 200. The use of any one of embodiments 182 to 199, wherein the obesity-related disorder is a cancer, a body weight disorder, or a metabolic disease or disorder.
Embodiment 201. The use of any one of embodiments 182 to 200, wherein the obesity-related disorder is a cancer, type II diabetes mellitus (T2DM), nonalcoholic steatohepatitis (NASH), hypertriglyceridemia, or cardiovascular disease.
Embodiment 202. A method of reducing appetite and/or body weight, comprising administering a GDF15 peptide to a subject, wherein the GDF15 peptide induces a biological response in a cell contacted with the GDF15 peptide, wherein the biological response is or can be detected by the method of any one of embodiments 1 to 124.
Embodiment 203. The method of embodiment 202, wherein the GDF15 peptide comprises the amino acid sequence of SEQ ID NO:13 or a functional variant thereof.
Embodiment 204. The method of embodiment 202, wherein the GDF15 peptide or functional variant has at least 80% amino acid sequence identity with the amino acid sequence of SEQ ID NO:13.
Embodiment 205. The method of embodiment 202, wherein the GDF15 peptide or functional variant has at least 90% amino acid sequence identity with the amino acid sequence of SEQ ID NO:13.
Embodiment 206. The method of any one of embodiments 202 to 205, wherein the GDF15 peptide comprises an affinity tag, a fusion, a conjugation, a PEGylation, and/or a glycosylation.
Embodiment 207. The method of any one of embodiments 202 to 206, wherein the GDF15 peptide is tagged with an amyloid-beta precursor protein tag, a histidine tag, a FLAG tag, or a myc tag.
Embodiment 208. The method of any one of embodiments 202 to 207, wherein the GDF15 peptide is fused to a human serum albumin, a mouse serum albumin, an immunoglobulin constant region, or an alpha-1-antitrypsin.
Embodiment 209. The method of any one of embodiments 202 to 208, wherein the GDF15 peptide is conjugated to a fatty acid.
Embodiment 210. The method of any one of embodiments 202 to 209, wherein the biological response is a signal transduction response.
Embodiment 211. The method of any one of embodiments 202 to 210, wherein the biological response is an increase or decrease in the expression or activity of a protein in the cell, as compared to the expression or activity of the same protein in a control cell that is not contacted with the GDF15 peptide.
Embodiment 212. The method of any one of embodiments 202 to 211, wherein the biological response is an increase or decrease in the expression or activity of an intracellular protein in one or more of the RET-ERK, RET-AKT, protein kinase C, JAK/STAT, JNK, p38, and RAC1 pathways.
Embodiment 213. The method of embodiment 211, wherein the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 214. The method of embodiment 211, wherein the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 215. The method of any one of embodiments 202 to 210, wherein the biological response is an increase or decrease in phosphorylation of a protein kinase in the cell, as compared to phosphorylation of the same protein kinase in a control cell that is not contacted with the GDF15 peptide.
Embodiment 216. The method of embodiment 215, wherein the protein kinase is an intracellular protein kinase, wherein the intracellular protein kinase is directly or indirectly phosphorylated by the cell surface receptor kinase.
Embodiment 217. The method of any one of embodiments 202 to 216, wherein the subject is overweight or obese.
Embodiment 218. The method of any one of embodiments 202 to 217, wherein the subject has a body mass index between 25 and 29.9.
Embodiment 219. The method of any one of embodiments 202 to 217, wherein the subject has a body mass index of 30 or higher.
Embodiment 220. Use of a GDF15 peptide in reducing appetite and/or body weight in a subject, wherein the GDF15 peptide induces a biological response in a cell contacted with the GDF15 peptide, wherein the biological response is or can be detected by the method of any one of embodiments 1 to 124.
Embodiment 221. The use of embodiment 220, wherein the GDF15 peptide comprises the amino acid sequence of SEQ ID NO:13 or a functional variant thereof.
Embodiment 222. The use of embodiment 220, wherein the GDF15 peptide or functional variant has at least 80% amino acid sequence identity with the amino acid sequence of SEQ ID NO:13.
Embodiment 223. The use of embodiment 220, wherein the GDF15 peptide or functional variant has at least 90% amino acid sequence identity with the amino acid sequence of SEQ ID NO:13.
Embodiment 224. The use of any one of embodiments 220 to 223, wherein the GDF15 peptide comprises an affinity tag, a fusion, a conjugation, a PEGylation, and/or a glycosylation.
Embodiment 225. The use of any one of embodiments 220 to 224, wherein the GDF15 peptide is tagged with an amyloid-beta precursor protein tag, a histidine tag, a FLAG tag, or a myc tag.
Embodiment 226. The use of any one of embodiments 220 to 225, wherein the GDF15 peptide is fused to a human serum albumin, a mouse serum albumin, an immunoglobulin constant region, or an alpha-1-antitrypsin.
Embodiment 227. The use of any one of embodiments 220 to 226, wherein the GDF15 peptide is conjugated to a fatty acid.
Embodiment 228. The use of any one of embodiments 220 to 227, wherein the biological response is a signal transduction response.
Embodiment 229. The use of any one of embodiments 220 to 228, wherein the biological response is an increase or decrease in the expression or activity of a protein in the cell, as compared to the expression or activity of the same protein in a control cell that is not contacted with the GDF15 peptide.
Embodiment 230. The use of any one of embodiments 220 to 229, wherein the biological response is an increase or decrease in the expression or activity of an intracellular protein in one or more of the RET-ERK, RET-AKT, protein kinase C, JAK/STAT, JNK, p38, and RAC1 pathways.
Embodiment 231. The use of embodiment 229, wherein the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 232. The use of embodiment 229, wherein the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 233. The use of any one of embodiments 220 to 228, wherein the biological response is an increase or decrease in phosphorylation of a protein kinase in the cell, as compared to phosphorylation of the same protein kinase in a control cell that is not contacted with the GDF15 peptide.
Embodiment 234. The use of embodiment 233, wherein the protein kinase is an intracellular protein kinase, wherein the intracellular protein kinase is directly or indirectly phosphorylated by the cell surface receptor kinase.
Embodiment 235. The use of any one of embodiments 220 to 234, wherein the subject is overweight or obese.
Embodiment 236. The use of any one of embodiments 220 to 235, wherein the subject has a body mass index between 25 and 29.9.
Embodiment 237. The use of any one of embodiments 220 to 235, wherein the subject has a body mass index of 30 or higher.
Embodiment 238. A soluble GFRAL comprising a GFRAL extracellular domain comprising domains D2 and D3.
Embodiment 239. The soluble GFRAL of embodiment 238, wherein the soluble GFRAL comprises a GFRAL extracellular domain lacking domain D1.
Embodiment 240. The soluble GFRAL of embodiment 238 or embodiment 239, wherein the soluble GFRAL further comprises a signal peptide.
Embodiment 241. The soluble GFRAL of any one of embodiments 238 to 240, wherein the soluble GFRAL comprises the amino acid sequence of SEQ ID NO:1 or a functional variant thereof.
Embodiment 242. The soluble GFRAL of any one of embodiments 238 to 240, wherein the soluble GFRAL or functional variant has at least 80% amino acid sequence identity with the amino acid sequence of SEQ ID NO:1.
Embodiment 243. The soluble GFRAL of any one of embodiments 238 to 240, wherein the soluble GFRAL or functional variant has at least 90% amino acid sequence identity with the amino acid sequence of SEQ ID NO:1.
Embodiment 244. The soluble GFRAL of any one of embodiments 238 to 240, wherein the soluble GFRAL comprises the amino acid sequence of SEQ ID NO:2 or a functional variant thereof.
Embodiment 245. The soluble GFRAL of any one of embodiments 238 to 244, wherein the soluble GFRAL further comprises (e.g., is fused to) an affinity tag.
Embodiment 246. The soluble GFRAL of embodiment 245, wherein the affinity tag comprises an amyloid-beta precursor protein tag, a histidine tag, a FLAG tag, or a myc tag.
Embodiment 247. A method of treating obesity or an obesity-related disorder, comprising administering a GDF15 peptide and a soluble GFRAL to a subject, wherein the GDF15 peptide induces a biological response in a cell contacted with the GDF15 peptide, and wherein the soluble GFRAL comprises a GFRAL extracellular domain comprising domains D2 and D3.
Embodiment 248. The method of embodiment 247, wherein the soluble GFRAL comprises a GFRAL extracellular domain lacking domain D1.
Embodiment 249. The method of embodiment 247 or embodiment 248, wherein the soluble GFRAL further comprises a signal peptide.
Embodiment 250. The method of any one of embodiments 247 to 249, wherein the soluble GFRAL comprises the amino acid sequence of SEQ ID NO:1 or a functional variant thereof.
Embodiment 251. The method of any one of embodiments 247 to 249, wherein the soluble GFRAL or functional variant has at least 80% amino acid sequence identity with the amino acid sequence of SEQ ID NO:1.
Embodiment 252. The method of any one of embodiments 247 to 249, wherein the soluble GFRAL or functional variant has at least 90% amino acid sequence identity with the amino acid sequence of SEQ ID NO:1.
Embodiment 253. The method of any one of embodiments 247 to 249, wherein the soluble GFRAL comprises the amino acid sequence of SEQ ID NO:2 or a functional variant thereof.
Embodiment 254. The method of any one of embodiments 247 to 253, wherein the soluble GFRAL further comprises (e.g., is fused to) an affinity tag.
Embodiment 255. The method of embodiment 254, wherein the affinity tag comprises an amyloid-beta precursor protein tag, a histidine tag, a FLAG tag, or a myc tag.
Embodiment 256. The method of any one of embodiments 247 to 255, wherein the GDF15 peptide comprises the amino acid sequence of SEQ ID NO:13 or a functional variant thereof.
Embodiment 257. The method of any one of embodiments 247 to 255, wherein the GDF15 peptide or functional variant has at least 80% amino acid sequence identity with the amino acid sequence of SEQ ID NO:13.
Embodiment 258. The method of any one of embodiments 247 to 255, wherein the GDF15 peptide or functional variant has at least 90% amino acid sequence identity with the amino acid sequence of SEQ ID NO:13.
Embodiment 259. The method of any one of embodiments 247 to 258, wherein the GDF15 peptide comprises an affinity tag, a fusion, a conjugation, a PEGylation, and/or a glycosylation.
Embodiment 260. The method of any one of embodiments 247 to 259, wherein the GDF15 peptide is tagged with an amyloid-beta precursor protein tag, a histidine tag, a FLAG tag, or a myc tag.
Embodiment 261. The method of any one of embodiments 247 to 260, wherein the GDF15 peptide is fused to a human serum albumin, a mouse serum albumin, an immunoglobulin constant region, or an alpha-1-antitrypsin.
Embodiment 262. The method of any one of embodiments 247 to 261, wherein the GDF15 peptide is conjugated to a fatty acid.
Embodiment 263. The method of any one of embodiments 247 to 262, wherein the GDF15 peptide and the soluble GFRAL are administered simultaneously.
Embodiment 264. The method of any one of embodiments 247 to 262, wherein the GDF15 peptide and the soluble GFRAL are administered sequentially.
Embodiment 265. The method of embodiment 263, wherein the GDF15 peptide and the soluble GFRAL are in the same composition.
Embodiment 266. The method of embodiment 265, wherein the GDF15 peptide and the soluble GFRAL are in a mixture.
Embodiment 267. The method of embodiment 265, wherein the GDF15 peptide and the soluble GFRAL are in a binary complex.
Embodiment 268. The method of any one of embodiments 247 to 267, wherein the biological response is a signal transduction response.
Embodiment 269. The method of any one of embodiments 247 to 268, wherein the biological response is an increase or decrease in the expression or activity of a protein in the cell, as compared to the expression or activity of the same protein in a control cell that is not contacted with the GDF15 peptide.
Embodiment 270. The method of any one of embodiments 247 to 269, wherein the biological response is an increase or decrease in the expression or activity of an intracellular protein in one or more of the RET-ERK, RET-AKT, protein kinase C, JAK/STAT, JNK, p38, and RAC1 pathways.
Embodiment 271. The method of embodiment 269, wherein the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 272. The method of embodiment 269, wherein the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 273. The method of any one of embodiments 247 to 268, wherein the biological response is an increase or decrease in phosphorylation of a protein kinase in the cell, as compared to phosphorylation of the same protein kinase in a control cell that is not contacted with the GDF15 peptide.
Embodiment 274. The method of embodiment 273, wherein the protein kinase is an intracellular protein kinase, wherein the intracellular protein kinase is directly or indirectly phosphorylated by the cell surface receptor kinase.
Embodiment 275. The method of any one of embodiments 247 to 274, wherein the subject is overweight or obese.
Embodiment 276. The method of any one of embodiments 247 to 275, wherein the subject has a body mass index between 25 and 29.9.
Embodiment 277. The method of any one of embodiments 247 to 275, wherein the subject has a body mass index of 30 or higher.
Embodiment 278. The method of any one of embodiments 247 to 277, wherein the obesity-related disorder is a cancer, a body weight disorder, or a metabolic disease or disorder.
Embodiment 279. The method of any one of embodiments 247 to 278, wherein the obesity-related disorder is a cancer, type II diabetes mellitus (T2DM), nonalcoholic steatohepatitis (NASH), hypertriglyceridemia, or cardiovascular disease.
Embodiment 280. Use of a GDF15 peptide and a soluble GFRAL in treating obesity or an obesity-related disorder in a subject, wherein the GDF15 peptide induces a biological response in a cell contacted with the GDF15 peptide, and wherein the soluble GFRAL comprises a GFRAL extracellular domain comprising domains D2 and D3.
Embodiment 281. The use of embodiment 280, wherein the soluble GFRAL comprises a GFRAL extracellular domain lacking domain D1.
Embodiment 282. The use of embodiment 280 or embodiment 281, wherein the soluble GFRAL further comprises a signal peptide.
Embodiment 283. The use of any one of embodiments 280 to 282, wherein the soluble GFRAL comprises the amino acid sequence of SEQ ID NO:1 or a functional variant thereof.
Embodiment 284. The use of any one of embodiments 280 to 282, wherein the soluble GFRAL or functional variant has at least 80% amino acid sequence identity with the amino acid sequence of SEQ ID NO:1.
Embodiment 285. The use of any one of embodiments 280 to 282, wherein the soluble GFRAL or functional variant has at least 90% amino acid sequence identity with the amino acid sequence of SEQ ID NO:1.
Embodiment 286. The use of any one of embodiments 280 to 282, wherein the soluble GFRAL comprises the amino acid sequence of SEQ ID NO:2 or a functional variant thereof.
Embodiment 287. The use of any one of embodiments 280 to 286, wherein the soluble GFRAL further comprises (e.g., is fused to) an affinity tag.
Embodiment 288. The use of embodiment 287, wherein the affinity tag comprises an amyloid-beta precursor protein tag, a histidine tag, a FLAG tag, or a myc tag.
Embodiment 289. The use of any one of embodiments 280 to 288, wherein the GDF15 peptide comprises the amino acid sequence of SEQ ID NO:13 or a functional variant thereof.
Embodiment 290. The use of any one of embodiments 280 to 288, wherein the GDF15 peptide or functional variant has at least 80% amino acid sequence identity with the amino acid sequence of SEQ ID NO:13.
Embodiment 291. The use of any one of embodiments 280 to 288, wherein the GDF15 peptide or functional variant has at least 90% amino acid sequence identity with the amino acid sequence of SEQ ID NO:13.
Embodiment 292. The use of any one of embodiments 280 to 291, wherein the GDF15 peptide comprises an affinity tag, a fusion, a conjugation, a PEGylation, and/or a glycosylation.
Embodiment 293. The use of any one of embodiments 280 to 292, wherein the GDF15 peptide is tagged with an amyloid-beta precursor protein tag, a histidine tag, a FLAG tag, or a myc tag.
Embodiment 294. The use of any one of embodiments 280 to 293, wherein the GDF15 peptide is fused to a human serum albumin, a mouse serum albumin, an immunoglobulin constant region, or an alpha-1-antitrypsin.
Embodiment 295. The use of any one of embodiments 280 to 294, wherein the GDF15 peptide is conjugated to a fatty acid.
Embodiment 296. The use of any one of embodiments 280 to 295, wherein the GDF15 peptide and the soluble GFRAL are administered simultaneously.
Embodiment 297. The use of any one of embodiments 280 to 295, wherein the GDF15 peptide and the soluble GFRAL are administered sequentially.
Embodiment 298. The use of embodiment 296, wherein the GDF15 peptide and the soluble GFRAL are in the same composition.
Embodiment 299. The use of embodiment 298, wherein the GDF15 peptide and the soluble GFRAL are in a mixture.
Embodiment 300. The use of embodiment 298, wherein the GDF15 peptide and the soluble GFRAL are in a binary complex.
Embodiment 301. The use of any one of embodiments 280 to 300, wherein the biological response is a signal transduction response.
Embodiment 302. The use of any one of embodiments 280 to 301, wherein the biological response is an increase or decrease in the expression or activity of a protein in the cell, as compared to the expression or activity of the same protein in a control cell that is not contacted with the GDF15 peptide.
Embodiment 303. The use of any one of embodiments 280 to 302, wherein the biological response is an increase or decrease in the expression or activity of an intracellular protein in one or more of the RET-ERK, RET-AKT, protein kinase C, JAK/STAT, JNK, p38, and RAC1 pathways.
Embodiment 304. The use of embodiment 302, wherein the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 305. The use of embodiment 302, wherein the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 306. The use of any one of embodiments 280 to 301, wherein the biological response is an increase or decrease in phosphorylation of a protein kinase in the cell, as compared to phosphorylation of the same protein kinase in a control cell that is not contacted with the GDF15 peptide.
Embodiment 307. The use of embodiment 306, wherein the protein kinase is an intracellular protein kinase, wherein the intracellular protein kinase is directly or indirectly phosphorylated by the cell surface receptor kinase.
Embodiment 308. The use of any one of embodiments 280 to 307, wherein the subject is overweight or obese.
Embodiment 309. The use of any one of embodiments 280 to 308, wherein the subject has a body mass index between 25 and 29.9.
Embodiment 310. The use of any one of embodiments 280 to 308, wherein the subject has a body mass index of 30 or higher.
Embodiment 311. The use of any one of embodiments 280 to 310, wherein the obesity-related disorder is a cancer, a body weight disorder, or a metabolic disease or disorder.
Embodiment 312. The use of any one of embodiments 280 to 311, wherein the obesity-related disorder is a cancer, type II diabetes mellitus (T2DM), nonalcoholic steatohepatitis (NASH), hypertriglyceridemia, or cardiovascular disease.
Embodiment 313. A method of reducing appetite and/or body weight, comprising administering a GDF15 peptide and a soluble GFRAL to a subject, wherein the GDF15 peptide induces a biological response in a cell contacted with the GDF15 peptide, and wherein the soluble GFRAL comprises a GFRAL extracellular domain comprising domains D2 and D3.
Embodiment 314. The method of embodiment 313, wherein the soluble GFRAL comprises a GFRAL extracellular domain lacking domain D1.
Embodiment 315. The method of embodiment 313 or embodiment 314, wherein the soluble GFRAL further comprises a signal peptide.
Embodiment 316. The method of any one of embodiments 313 to 315, wherein the soluble GFRAL comprises the amino acid sequence of SEQ ID NO:1 or a functional variant thereof.
Embodiment 317. The method of any one of embodiments 313 to 315, wherein the soluble GFRAL or functional variant has at least 80% amino acid sequence identity with the amino acid sequence of SEQ ID NO:1.
Embodiment 318. The method of any one of embodiments 313 to 315, wherein the soluble GFRAL or functional variant has at least 90% amino acid sequence identity with the amino acid sequence of SEQ ID NO:1.
Embodiment 319. The method of any one of embodiments 313 to 315, wherein the soluble GFRAL comprises the amino acid sequence of SEQ ID NO:2 or a functional variant thereof.
Embodiment 320. The method of any one of embodiments 313 to 319, wherein the soluble GFRAL further comprises (e.g., is fused to) an affinity tag.
Embodiment 321. The method of embodiment 320, wherein the affinity tag comprises an amyloid-beta precursor protein tag, a histidine tag, a FLAG tag, or a myc tag.
Embodiment 322. The method of any one of embodiments 313 to 321, wherein the GDF15 peptide comprises the amino acid sequence of SEQ ID NO:13 or a functional variant thereof.
Embodiment 323. The method of any one of embodiments 313 to 321, wherein the GDF15 peptide or functional variant has at least 80% amino acid sequence identity with the amino acid sequence of SEQ ID NO:13.
Embodiment 324. The method of any one of embodiments 313 to 321, wherein the GDF15 peptide or functional variant has at least 90% amino acid sequence identity with the amino acid sequence of SEQ ID NO:13.
Embodiment 325. The method of any one of embodiments 313 to 324, wherein the GDF15 peptide comprises an affinity tag, a fusion, a conjugation, a PEGylation, and/or a glycosylation.
Embodiment 326. The method of any one of embodiments 313 to 325, wherein the GDF15 peptide is tagged with an amyloid-beta precursor protein tag, a histidine tag, a FLAG tag, or a myc tag.
Embodiment 327. The method of any one of embodiments 313 to 326, wherein the GDF15 peptide is fused to a human serum albumin, a mouse serum albumin, an immunoglobulin constant region, or an alpha-1-antitrypsin.
Embodiment 328. The method of any one of embodiments 313 to 327, wherein the GDF15 peptide is conjugated to a fatty acid.
Embodiment 329. The method of any one of embodiments 313 to 328, wherein the GDF15 peptide and the soluble GFRAL are administered simultaneously.
Embodiment 330. The method of any one of embodiments 313 to 328, wherein the GDF15 peptide and the soluble GFRAL are administered sequentially.
Embodiment 331. The method of embodiment 329, wherein the GDF15 peptide and the soluble GFRAL are in the same composition.
Embodiment 332. The method of embodiment 331, wherein the GDF15 peptide and the soluble GFRAL are in a mixture.
Embodiment 333. The method of embodiment 331, wherein the GDF15 peptide and the soluble GFRAL are in a binary complex.
Embodiment 334. The method of any one of embodiments 313 to 333, wherein the biological response is a signal transduction response.
Embodiment 335. The method of any one of embodiments 313 to 334, wherein the biological response is an increase or decrease in the expression or activity of a protein in the cell, as compared to the expression or activity of the same protein in a control cell that is not contacted with the GDF15 peptide.
Embodiment 336. The method of any one of embodiments 313 to 335, wherein the biological response is an increase or decrease in the expression or activity of an intracellular protein in one or more of the RET-ERK, RET-AKT, protein kinase C, JAK/STAT, JNK, p38, and RAC1 pathways.
Embodiment 337. The method of embodiment 335, wherein the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 338. The method of embodiment 335, wherein the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 339. The method of any one of embodiments 313 to 334, wherein the biological response is an increase or decrease in phosphorylation of a protein kinase in the cell, as compared to phosphorylation of the same protein kinase in a control cell that is not contacted with the GDF15 peptide.
Embodiment 340. The method of embodiment 339, wherein the protein kinase is an intracellular protein kinase, wherein the intracellular protein kinase is directly or indirectly phosphorylated by the cell surface receptor kinase.
Embodiment 341. The method of any one of embodiments 313 to 340, wherein the subject is overweight or obese.
Embodiment 342. The method of any one of embodiments 313 to 341, wherein the subject has a body mass index between 25 and 29.9.
Embodiment 343. The method of any one of embodiments 313 to 341, wherein the subject has a body mass index of 30 or higher.
Embodiment 344. Use of a GDF15 peptide and a soluble GFRAL in reducing appetite and/or body weight in a subject, wherein the GDF15 peptide induces a biological response in a cell contacted with the GDF15 peptide, and wherein the soluble GFRAL comprises a GFRAL extracellular domain comprising domains D2 and D3.
Embodiment 345. The use of embodiment 344, wherein the soluble GFRAL comprises a GFRAL extracellular domain lacking domain D1.
Embodiment 346. The use of embodiment 344 or embodiment 345, wherein the soluble GFRAL further comprises a signal peptide.
Embodiment 347. The use of any one of embodiments 344 to 346, wherein the soluble GFRAL comprises the amino acid sequence of SEQ ID NO:1 or a functional variant thereof.
Embodiment 348. The use of any one of embodiments 344 to 346, wherein the soluble GFRAL or functional variant has at least 80% amino acid sequence identity with the amino acid sequence of SEQ ID NO:1.
Embodiment 349. The use of any one of embodiments 344 to 346, wherein the soluble GFRAL or functional variant has at least 90% amino acid sequence identity with the amino acid sequence of SEQ ID NO:1.
Embodiment 350. The use of any one of embodiments 344 to 346, wherein the soluble GFRAL comprises the amino acid sequence of SEQ ID NO:2 or a functional variant thereof.
Embodiment 351. The use of any one of embodiments 344 to 350, wherein the soluble GFRAL further comprises (e.g., is fused to) an affinity tag.
Embodiment 352. The use of embodiment 351, wherein the affinity tag comprises an amyloid-beta precursor protein tag, a histidine tag, a FLAG tag, or a myc tag.
Embodiment 353. The use of any one of embodiments 344 to 352, wherein the GDF15 peptide comprises the amino acid sequence of SEQ ID NO:13 or a functional variant thereof.
Embodiment 354. The use of any one of embodiments 344 to 352, wherein the GDF15 peptide or functional variant has at least 80% amino acid sequence identity with the amino acid sequence of SEQ ID NO:13.
Embodiment 355. The use of any one of embodiments 344 to 352, wherein the GDF15 peptide or functional variant has at least 90% amino acid sequence identity with the amino acid sequence of SEQ ID NO:13.
Embodiment 356. The use of any one of embodiments 344 to 355, wherein the GDF15 peptide comprises an affinity tag, a fusion, a conjugation, a PEGylation, and/or a glycosylation.
Embodiment 357. The use of any one of embodiments 344 to 356, wherein the GDF15 peptide is tagged with an amyloid-beta precursor protein tag, a histidine tag, a FLAG tag, or a myc tag.
Embodiment 358. The use of any one of embodiments 344 to 357, wherein the GDF15 peptide is fused to a human serum albumin, a mouse serum albumin, an immunoglobulin constant region, or an alpha-1-antitrypsin.
Embodiment 359. The use of any one of embodiments 344 to 358, wherein the GDF15 peptide is conjugated to a fatty acid.
Embodiment 360. The use of any one of embodiments 344 to 359, wherein the GDF15 peptide and the soluble GFRAL are administered simultaneously.
Embodiment 361. The use of any one of embodiments 344 to 359, wherein the GDF15 peptide and the soluble GFRAL are administered sequentially.
Embodiment 362. The use of embodiment 360, wherein the GDF15 peptide and the soluble GFRAL are in the same composition.
Embodiment 363. The use of embodiment 362, wherein the GDF15 peptide and the soluble GFRAL are in a mixture.
Embodiment 364. The use of embodiment 362, wherein the GDF15 peptide and the soluble GFRAL are in a binary complex.
Embodiment 365. The use of any one of embodiments 344 to 364, wherein the biological response is a signal transduction response.
Embodiment 366. The use of any one of embodiments 344 to 365, wherein the biological response is an increase or decrease in the expression or activity of a protein in the cell, as compared to the expression or activity of the same protein in a control cell that is not contacted with the GDF15 peptide.
Embodiment 367. The use of any one of embodiments 344 to 366, wherein the biological response is an increase or decrease in the expression or activity of an intracellular protein in one or more of the RET-ERK, RET-AKT, protein kinase C, JAK/STAT, JNK, p38, and RAC1 pathways.
Embodiment 368. The use of embodiment 366, wherein the protein is an intracellular protein in the RET-ERK pathway selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 369. The use of embodiment 366, wherein the protein is an intracellular protein the RET-AKT pathway selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 370. The use of any one of embodiments 344 to 365, wherein the biological response is an increase or decrease in phosphorylation of a protein kinase in the cell, as compared to phosphorylation of the same protein kinase in a control cell that is not contacted with the GDF15 peptide.
Embodiment 371. The use of embodiment 370, wherein the protein kinase is an intracellular protein kinase, wherein the intracellular protein kinase is directly or indirectly phosphorylated by the cell surface receptor kinase.
Embodiment 372. The use of any one of embodiments 344 to 371, wherein the subject is overweight or obese.
Embodiment 373. The use of any one of embodiments 344 to 372, wherein the subject has a body mass index between 25 and 29.9.
Embodiment 374. The use of any one of embodiments 344 to 372, wherein the subject has a body mass index of 30 or higher.
Embodiment 375. A method of identifying an agent capable of modulating GDF15 activity, comprising:

(a) contacting the cell of any one of embodiments 125 to 160 with the agent and a GDF15 peptide; and

(b) detecting a biological response in the contacted cell, wherein the agent is determined to modulate GDF15 activity if the biological response in the contacted cell is increased or decreased relative to the biological response in a cell contacted with the GDF15 peptide in the absence of the agent.

Embodiment 376. The method of embodiment 375, wherein the agent is an antibody.
Embodiment 377. The method of embodiment 375 or embodiment 376, wherein the agent is an anti-GDF15 antibody.
Embodiment 378. The method of embodiment 375 or embodiment 376, wherein the agent is an anti-GFRAL antibody.
Embodiment 379. The method of any one of embodiments 375 to 378, wherein the biological response is an increase or decrease in the expression, activity, or phosphorylation level of an intracellular protein in one or more of the RET-ERK, RET-AKT, protein kinase C, JAK/STAT, JNK, p38, and RAC1 pathways.
Embodiment 380. The method of embodiment 379, wherein the intracellular protein is in the RET-ERK pathway and is selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 381. The method of embodiment 379, wherein the intracellular protein is in the RET-AKT pathway and is selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 382. The method of any one of embodiments 375 to 381, wherein the GDF15 peptide comprises the amino acid sequence of SEQ ID NO:13 or a functional variant thereof.
Embodiment 383. The method of any one of embodiments 375 to 381, wherein the GDF15 peptide or functional variant has at least 80% amino acid sequence identity with the amino acid sequence of SEQ ID NO:13.
Embodiment 384. The method of any one of embodiments 375 to 381, wherein the GDF15 peptide or functional variant has at least 90% amino acid sequence identity with the amino acid sequence of SEQ ID NO:13.
Embodiment 385. The method of any one of embodiments 375 to 384, wherein the GDF15 peptide comprises an affinity tag, a fusion, a conjugation, a PEGylation, and/or a glycosylation.
Embodiment 386. The method of any one of embodiments 375 to 385, wherein the GDF15 peptide is tagged with an amyloid-beta precursor protein tag, a histidine tag, a FLAG tag, or a myc tag.
Embodiment 387. The method of any one of embodiments 375 to 386, wherein the GDF15 peptide is fused to a human serum albumin, a mouse serum albumin, an immunoglobulin constant region, or an alpha-1-antitrypsin.
Embodiment 388. The method of any one of embodiments 375 to 387, wherein the GDF15 peptide is conjugated to a fatty acid.
Embodiment 389. A method of identifying an agent capable of modulating GDF15 activity, comprising:

(a) providing a cell that expresses a cell surface receptor kinase;

(b) contacting the cell with a GDF15 peptide and a soluble GFRAL;

(c) contacting the cell with the agent; and

(d) detecting a biological response in the contacted cell,

wherein the soluble GFRAL comprises a GFRAL extracellular domain comprising domains D2 and D3 and lacks domain D1.
Embodiment 390. The method of embodiment 389, wherein the agent is determined to modulate or increase GDF15 activity if the biological response in the contacted cell is increased in the presence of the GDF15 peptide, the soluble GFRAL, and the agent relative to the biological response in a cell contacted with the GDF15 peptide and the soluble GFRAL in the absence of the agent.
Embodiment 391. The method of embodiment 389, wherein the agent is determined to modulate or decrease GDF15 activity if the biological response in the contacted cell is decreased in the presence of the GDF15 peptide, the soluble GFRAL, and the agent relative to the biological response in a cell contacted with the GDF15 peptide and the soluble GFRAL in the absence of the agent.
Embodiment 392. The method of any one of embodiments 389 to 391, wherein the agent is an antibody.
Embodiment 393. The method of any one of embodiments 389 to 392, wherein the agent is an anti-GDF15 antibody.
Embodiment 394. The method of any one of embodiments 389 to 392, wherein the agent is an anti-GFRAL antibody.
Embodiment 395. The method of any one of embodiments 389 to 394, wherein the biological response is an increase or decrease in the expression, activity, or phosphorylation level of an intracellular protein in one or more of the RET-ERK, RET-AKT, protein kinase C, JAK/STAT, JNK, p38, and RAC1 pathways.
Embodiment 396. The method of embodiment 395, wherein intracellular protein is in the RET-ERK pathway and is selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.
Embodiment 397. The method of embodiment 395, wherein the intracellular protein is in the RET-AKT pathway and is selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.
Embodiment 398. The method of any one of embodiments 389 to 397, wherein the soluble GFRAL comprises the amino acid sequence of SEQ ID NO:1 or a functional variant thereof.
Embodiment 399. The method of any one of embodiments 389 to 397, wherein the soluble GFRAL or functional variant has at least 80% amino acid sequence identity with the amino acid sequence of SEQ ID NO:1.
Embodiment 400. The method of any one of embodiments 389 to 397, wherein the soluble GFRAL or functional variant has at least 90% amino acid sequence identity with the amino acid sequence of SEQ ID NO:1.
Embodiment 401. The method of any one of embodiments 389 to 400, wherein the soluble GFRAL further comprises (e.g., is fused to) an affinity tag.
Embodiment 402. The method of embodiment 401, wherein the affinity tag comprises an amyloid-beta precursor protein tag, a histidine tag, a FLAG tag, or a myc tag.
Embodiment 403. The method of any one of embodiments 389 to 402, wherein the GDF15 peptide comprises the amino acid sequence of SEQ ID NO:13 or a functional variant thereof.
Embodiment 404. The method of any one of embodiments 389 to 402, wherein the GDF15 peptide or functional variant has at least 80% amino acid sequence identity with the amino acid sequence of SEQ ID NO:13.
Embodiment 405. The method of any one of embodiments 389 to 402, wherein the GDF15 peptide or functional variant has at least 90% amino acid sequence identity with the amino acid sequence of SEQ ID NO:13.
Embodiment 406. The method of any one of embodiments 389 to 405, wherein the GDF15 peptide comprises an affinity tag, a fusion, a conjugation, a PEGylation, and/or a glycosylation.
Embodiment 407. The method of any one of embodiments 389 to 406, wherein the GDF15 peptide is tagged with an amyloid-beta precursor protein tag, a histidine tag, a FLAG tag, or a myc tag.
Embodiment 408. The method of any one of embodiments 389 to 407, wherein the GDF15 peptide is fused to a human serum albumin, a mouse serum albumin, an immunoglobulin constant region, or an alpha-1-antitrypsin.
Embodiment 409. The method of any one of embodiments 389 to 408, wherein the GDF15 peptide is conjugated to a fatty acid.
Embodiment 410. A method of producing a pharmaceutical composition comprising an agent, comprising:

(a) identifying an agent capable of modulating GDF15 activity by the method of any one of embodiments 375 to 409; and

(b) formulating the agent in a pharmaceutical composition.

Embodiment 411. The method of embodiment 410, wherein the agent is an antibody.
Embodiment 412. The method of embodiment 410 or embodiment 411, wherein the agent is an anti-GDF15 antibody.
Embodiment 413. The method of embodiment 410 or embodiment 411, wherein the agent is an anti-GFRAL antibody.
Embodiment 414. A method of treating obesity or an obesity-related disorder in a subject, comprising:

(a) identifying an agent capable of modulating GDF15 activity by the method of any one of embodiments 375 to 409; and

(b) administering the agent to the subject.

Embodiment 415. The method of embodiment 414, wherein the agent is an antibody. Embodiment 416. The method of embodiment 414 or embodiment 415, wherein the agent is an anti-GDF15 antibody.
Embodiment 417. The method of embodiment 414 or embodiment 415, wherein the agent is an anti-GFRAL antibody.
Embodiment 418. The method of any one of embodiments 414 to 417, wherein the subject is overweight or obese.
Embodiment 419. The method of any one of embodiments 414 to 418, wherein the subject has a body mass index between 25 and 29.9.
Embodiment 420. The method of any one of embodiments 414 to 418, wherein the subject has a body mass index of 30 or higher.
Embodiment 421. The method of any one of embodiments 414 to 420, wherein the obesity-related disorder is a cancer, a body weight disorder, or a metabolic disease or disorder.
Embodiment 422. The method of any one of embodiments 414 to 421, wherein the obesity-related disorder is a cancer, type II diabetes mellitus (T2DM), nonalcoholic steatohepatitis (NASH), hypertriglyceridemia, or cardiovascular disease.
Embodiment 423. A method of reducing appetite and/or body weight in a subject, comprising:

(a) identifying an agent capable of modulating GDF15 activity by the method of any one of embodiments 375 to 409; and

(b) administering the agent to the subject.

Embodiment 424. The method of embodiment 423, wherein the agent is an antibody.
Embodiment 425. The method of embodiment 423 or embodiment 424, wherein the agent is an anti-GDF15 antibody.
Embodiment 426. The method of embodiment 423 or embodiment 424, wherein the agent is an anti-GFRAL antibody.
Embodiment 427. The method of any one of embodiments 423 to 426, wherein the subject is overweight or obese.
Embodiment 428. The method of any one of embodiments 423 to 427, wherein the subject has a body mass index between 25 and 29.9.
Embodiment 429. The method of any one of embodiments 423 to 427, wherein the subject has a body mass index of 30 or higher.

1. A method of detecting the activity of a GDF15 peptide, comprising:
(i)
(a) providing a cell that expresses a cell surface receptor kinase;
(b) contacting the cell with the GDF15 peptide and a soluble GFRAL, wherein the soluble GFRAL comprises a GFRAL extracellular domain comprising domains D2 and D3; and
(c) detecting a biological response in the contacted cell; or
(ii)
(a) providing a cell that expresses a cell surface receptor kinase and a GFRAL extracellular domain comprising domains D2 and D3;
(b) contacting the cell with the GDF15 peptide; and
(c) detecting a biological response in the contacted cell.

2. The method of claim 1, wherein the GFRAL extracellular domain lacks domain D1.

3. The method of claim 1 or 2 providing a cell that expresses a cell surface receptor kinase and a GFRAL extracellular domain, wherein
(i) the GFRAL extracellular domain is a soluble GFRAL extracellular domain, or
(ii) the GFRAL extracellular domain is attached to the cell surface by a tether.

4. The method of claim 3, wherein the tether
(i) is a GFRAL transmembrane domain or a functional fragment thereof;
(ii) comprises the amino acid sequence of SEQ ID NO:18 or a functional variant thereof;
(iii) is a heterologous transmembrane domain fused to the GFRAL extracellular domain;
(iv) is a glycophosphatidylinositol (GPI);
(v) comprises the amino acid sequence of SEQ ID NO:19 or a functional variant thereof, SEQ ID NO:20 or a functional variant thereof, or SEQ ID NO:21 or a functional variant thereof;
(vi) is a membrane-inserting sequence;
(vii) comprises the amino acid sequence of SEQ ID NO:22 or a functional variant thereof, or SEQ ID NO:23 or a functional variant thereof; or
(viii) is a membrane-inserting fatty acid.

5. The method of any one of claims 1 to 4, wherein the GFRAL extracellular domain further comprises a signal peptide.

6. The method of any one of claims 1 to 5, wherein the GFRAL extracellular domain is tagged with an affinity tag selected from an amyloid-beta precursor protein tag, a histidine tag, a FLAG tag, and a myc tag.

7. The method of any one of claims 1 to 6, wherein the GFRAL extracellular domain
(i) comprises the amino acid sequence of SEQ ID NO:1 or a functional variant thereof;
(ii) has at least 80% amino acid sequence identity with the amino acid sequence of SEQ ID NO:1;
(iii) has at least 90% amino acid sequence identity with the amino acid sequence of SEQ ID NO:1;
(iv) comprises the amino acid sequence of SEQ ID NO:2 or a functional variant thereof;
(v) has at least 80% amino acid sequence identity with the amino acid sequence of SEQ ID NO:2;
(vi) has at least 90% amino acid sequence identity with the amino acid sequence of SEQ ID NO:2;
(vii) comprises the amino acid sequence of SEQ ID NO:3 or a functional variant thereof; or
(viii) comprises the amino acid sequence of SEQ ID NO:25 or a functional variant thereof.

8. The method of any one of claims 1 to 7, wherein the GDF15 peptide or functional variant thereof
(i) comprises the amino acid sequence of SEQ ID NO:13, 14, 15, 16 or 17, or a functional variant thereof;
(ii) has at least 80% amino acid sequence identity with the amino acid sequence of SEQ ID NO:13; or
(iii) has at least 90% amino acid sequence identity with the amino acid sequence of SEQ ID NO:13.

9. The method of any one of claims 1 to 8, wherein the GDF15 peptide
(i) is tagged with an affinity tag selected from an amyloid-beta precursor protein tag, a histidine tag, a FLAG tag, and a myc tag;
(ii) is fused to a human serum albumin, a mouse serum albumin, an immunoglobulin constant region, or an alpha-1-antitrypsin;
(iii) is conjugated to a fatty acid;
(iv) has a PEGylation; and/or
(v) has a glycosylation.

10. The method of any one of claims 1 to 9, wherein the cell surface receptor kinase is
(i) an endogenous cell surface receptor kinase;
(ii) an exogenous cell surface receptor kinase; and/or
(iii) a RET receptor tyrosine kinase.

11. The method of any one of claims 1 to 10, wherein the cell does not express
(i) endogenous GFRAL;
(ii) full length GFRAL; and/or
(iii) endogenous GDF15.

12. The method of any one of claims 1 to 11, wherein the cell is a GDF15 knockout (KO) cell comprising an inoperative GDF15 gene.

13. The method of any one of claims 1 to 12, wherein the biological response
(i) is induced when the GDF15 peptide, the soluble GFRAL or the GFRAL extracellular domain, and the cell surface receptor kinase form a ternary complex;
(ii) is not induced in a cell contacted with the GDF15 peptide in the absence of the soluble GFRAL; and/or
(iii) is an increase or decrease in the expression or activity of a protein in the cell, as compared to the expression or activity of the same protein in a control cell that is not contacted with the GDF15 peptide and/or the soluble GFRAL.

14. The method of any one of claims 1 to 13, wherein the biological response is an increase or decrease in the expression or activity of an intracellular protein in one or more of the RET-ERK, RET-AKT, protein kinase C, JAK/STAT, JNK, p38, and RAC1 pathways.

15. The method of claim 14, wherein the cell surface receptor kinase is a RET receptor tyrosine kinase and the protein is
(i) an intracellular protein in the RET-ERK pathway selected from ERK1, ERK2, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2, or any downstream targets thereof; or
(ii) is an intracellular protein the RET-AKT pathway selected from AKT1, AKT2, AKT3, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR, or any downstream targets thereof.

16. The method of any one of claims 1 to 15, wherein the biological response is an increase or decrease in phosphorylation of a protein kinase in the cell, as compared to phosphorylation of the same protein kinase in a control cell that is not contacted with the GDF15 peptide and/or the soluble GFRAL.

17. The method of claim 16, wherein
(i) the protein kinase is the cell surface receptor kinase;
(ii) the protein kinase and/or cell surface receptor kinase is a RET receptor tyrosine kinase; or
(iii) the protein kinase is an intracellular protein kinase, wherein the intracellular protein kinase is directly or indirectly phosphorylated by the cell surface receptor kinase.

18. The method of claim 16 or 17, wherein the protein kinase is
(i) an intracellular protein kinase in the RET-ERK pathway selected from of ERK1, ERK2, JAK1, JAK2, RAF, MEK1, MEK2, RSK1, RSK2, RSK3, MNK1, MNK2, MSK1, and MSK2, or any downstream targets thereof; or
(ii) an intracellular protein kinase in the RET-AKT pathway selected from AKT1, AKT2, AKT3, SRC, JAK1, JAK2, PI3K, PDK1, MLK3, ASK1, GSK3alpha, GSK3beta, and mTOR, or any downstream targets thereof.

19. An isolated and modified cell for detecting the activity of a GDF15 peptide, wherein the cell expresses a GFRAL extracellular domain comprising domains D2 and D3 and a cell surface receptor kinase.

20. The cell of claim 19, wherein the GFRAL extracellular domain lacks domain D1.

21. The cell of claim 19 or 20, wherein the GFRAL extracellular domain is
(i) a soluble GFRAL extracellular domain; or
(ii) attached to the cell surface by a tether.

22. The cell of claim 21, wherein the tether
(i) is a GFRAL transmembrane domain or a functional fragment thereof;
(ii) comprises the amino acid sequence of SEQ ID NO:18 or a functional variant thereof;
(iii) is a heterologous transmembrane domain fused to the GFRAL extracellular domain;
(iv) is a glycophosphatidylinositol (GPI);
(v) comprises the amino acid sequence of SEQ ID NO:19 or a functional variant thereof, SEQ ID NO:20 or a functional variant thereof, or SEQ ID NO:21 or a functional variant thereof;
(vi) is a membrane-inserting sequence;
(vii) comprises the amino acid sequence of SEQ ID NO:22 or a functional variant thereof, or SEQ ID NO:23 or a functional variant thereof; or
(viii) is a membrane-inserting fatty acid.

23. The cell of any one of claims 19 to 22, wherein the GFRAL extracellular domain further comprises a signal peptide.

24. The cell of any one of claims 19 to 23, wherein the GFRAL extracellular domain is tagged with an affinity tag selected from an amyloid-beta precursor protein tag, a histidine tag, a FLAG tag, and a myc tag.

25. The cell of any one of claims 19 to 24, wherein the GFRAL extracellular domain
(i) comprises the amino acid sequence of SEQ ID NO:1 or a functional variant thereof;
(ii) has at least 80% amino acid sequence identity with the amino acid sequence of SEQ ID NO:1;
(iii) has at least 90% amino acid sequence identity with the amino acid sequence of SEQ ID NO:1;
(iv) comprises the amino acid sequence of SEQ ID NO:2 or a functional variant thereof;
(v) has at least 80% amino acid sequence identity with the amino acid sequence of SEQ ID NO:2;
(vi) has at least 90% amino acid sequence identity with the amino acid sequence of SEQ ID NO:2;
(vii) comprises the amino acid sequence of SEQ ID NO:3 or a functional variant thereof; or
(viii) comprises the amino acid sequence of SEQ ID NO:25 or a functional variant thereof.

26. The cell of any one of claims 19 to 25, wherein the cell surface receptor kinase is
(i) an endogenous cell surface receptor kinase;
(ii) an exogenous cell surface receptor kinase; and/or
(iii) a RET receptor tyrosine kinase.

27. The cell of any one of claims 19 to 26, wherein the cell does not express
(i) endogenous GFRAL;
(ii) full length GFRAL; and/or
(iii) endogenous GDF15.

28. The cell of any one of claims 19 to 27, wherein the cell is a GDF15 knockout (KO) cell comprising an inoperative GDF15 gene.

29. The cell of any one of claims 19 to 28, wherein the cell is selected from a mammalian cell, a human cell, an MCF7 cell, an SH-SY5Y cell, and an HEK293A-GDF15 KO cell.

30. A kit for determining the activity of a GDF15 peptide, wherein the kit comprises the cell of any one of claims 19 to 29 for contacting with the GDF15 peptide; and a means of detecting a biological response in the contacted cell.

31. A soluble GFRAL comprising a GFRAL extracellular domain comprising domains D2 and D3.

32. The soluble GFRAL of claim 31, wherein the GFRAL extracellular domain lacks domain D1.

33. The soluble GFRAL of claim 31 or 32, wherein the GFRAL extracellular domain further comprises a signal peptide.

34. The soluble GFRAL of any one of claims 31 to 33, wherein the GFRAL extracellular domain is tagged with an affinity tag selected from an amyloid-beta precursor protein tag, a histidine tag, a FLAG tag, and a myc tag.

35. The soluble GFRAL of any one of claims 31 to 34, wherein the GFRAL extracellular domain
(i) comprises the amino acid sequence of SEQ ID NO:1 or a functional variant thereof;
(ii) has at least 80% amino acid sequence identity with the amino acid sequence of SEQ ID NO:1;
(iii) has at least 90% amino acid sequence identity with the amino acid sequence of SEQ ID NO:1;
(iv) comprises the amino acid sequence of SEQ ID NO:2 or a functional variant thereof;
(v) has at least 80% amino acid sequence identity with the amino acid sequence of SEQ ID NO:2;
(vi) has at least 90% amino acid sequence identity with the amino acid sequence of SEQ ID NO:2;
(vii) comprises the amino acid sequence of SEQ ID NO:3 or a functional variant thereof; or
(viii) comprises the amino acid sequence of SEQ ID NO:25 or a functional variant thereof.

36. A method of identifying an agent capable of modulating GDF15 activity, wherein the method comprises
(a) contacting the cell of any one of claims 19 to 29 with the agent and a GDF15 peptide; and
(b) detecting a biological response in the contacted cell,
wherein the agent is determined to modulate GDF15 activity if the biological response in the contacted cell is increased or decreased relative to the biological response in a cell contacted with the GDF15 peptide in the absence of the agent.

37. A method of identifying an agent capable of modulating GDF15 activity, comprising:
(a) providing a cell that expresses a cell surface receptor kinase;
(b) contacting the cell with a GDF15 peptide and a soluble GFRAL, wherein the soluble GFRAL comprises a GFRAL extracellular domain comprising domains D2 and D3 and lacks domain D1;
(c) contacting the cell with the agent; and
(d) detecting a biological response in the contacted cell,
wherein the agent is determined to
(i) modulate or increase GDF15 activity if the biological response in the contacted cell is increased in the presence of the GDF15 peptide, the soluble GFRAL, and the agent relative to the biological response in a cell contacted with the GDF15 peptide and the soluble GFRAL in the absence of the agent; or
(ii) modulate or decrease GDF15 activity if the biological response in the contacted cell is decreased in the presence of the GDF15 peptide, the soluble GFRAL, and the agent relative to the biological response in a cell contacted with the GDF15 peptide and the soluble GFRAL in the absence of the agent.

38. The method of claim 37, wherein the GFRAL extracellular domain is tagged with an affinity tag selected from an amyloid-beta precursor protein tag, a histidine tag, a FLAG tag, and a myc tag.

39. The method of claim 37 or 38, wherein the GFRAL extracellular domain
(i) comprises the amino acid sequence of SEQ ID NO:1 or a functional variant thereof;
(ii) has at least 80% amino acid sequence identity with the amino acid sequence of SEQ ID NO:1;
(iii) has at least 90% amino acid sequence identity with the amino acid sequence of SEQ ID NO:1;
(iv) comprises the amino acid sequence of SEQ ID NO:2 or a functional variant thereof;
(v) has at least 80% amino acid sequence identity with the amino acid sequence of SEQ ID NO:2;
(vi) has at least 90% amino acid sequence identity with the amino acid sequence of SEQ ID NO:2;
(vii) comprises the amino acid sequence of SEQ ID NO:3 or a functional variant thereof; or
(viii) comprises the amino acid sequence of SEQ ID NO:25 or a functional variant thereof.

40. The method of any one of claims 36 to 39, wherein the agent is an antibody selected from an anti-GDF15 antibody and an anti-GFRAL antibody.

41. The method of any one of claims 36 to 40, wherein the biological response is an increase or decrease in the expression, activity, or phosphorylation level of an intracellular protein in one or more of the RET-ERK, RET-AKT, protein kinase C, JAK/STAT, JNK, p38, and RAC1 pathways.

42. The method of claim 41, wherein the intracellular protein is in the RET-ERK pathway and is selected from ERK, SHC1, FRS2, GRB2, GAB1, GAB2, SOS, SHANK3, GRB7, GRB10, JAK1, JAK2, RAF, RAS, MEK1, MEK2, RSK1, RSK2, RSK3, MNK1, MNK2, MSK1, and MSK2.

43. The method of claim 41, wherein the intracellular protein is in the RET-AKT pathway and is selected from AKT, SRC, SHC1, GRB2, CBL, GAB1, GAB2, SHANK3, JAK1, JAK2, RAS, PI3K, PDK1, YAP, BAD, Caspase-9, FoxO1, FoxO3, FoxO4, IKKalpha, CREB, MDM2, MLK3, ASK1, p21Cip1, p27Kip1, GSK3alpha, GSK3beta, and mTOR.

44. The method of any one of claims 36 to 43, wherein the GDF15 peptide or functional variant thereof
(i) comprises the amino acid sequence of SEQ ID NO:13, 14, 15, 16 or 17, or a functional variant thereof;
(ii) has at least 80% amino acid sequence identity with the amino acid sequence of SEQ ID NO:13; or
(iii) has at least 90% amino acid sequence identity with the amino acid sequence of SEQ ID NO:13.

45. The method of any one of claims 36 to 44, wherein the GDF15 peptide
(i) is tagged with an affinity tag selected from an amyloid-beta precursor protein tag, a histidine tag, a FLAG tag, and a myc tag;
(ii) is fused to a human serum albumin, a mouse serum albumin, an immunoglobulin constant region, or an alpha-1-antitrypsin;
(iii) is conjugated to a fatty acid;
(iv) has a PEGylation; and/or
(v) has a glycosylation.


SEQUENCE LISTING (26 sequences)

SEQ ID NO: 1 - protein, 211 aa, Homo sapiens
GFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRK

SEQ ID NO: 2 - protein, 227 aa, Homo sapiens
MPLLLLLPLLWAGALAGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRK

SEQ ID NO: 3 - protein, 235 aa, Artificial Sequence
  source: /note=“Description of Artificial Sequence Synthetic polypeptide”
MPLLLLLPLLWAGALAGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKGSEFRHDS

SEQ ID NO: 4 - protein, 332 aa, Homo sapiens
QTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGE

SEQ ID NO: 5 - protein, 348 aa, Homo sapiens
MPLLLLLPLLWAGALAQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGE

SEQ ID NO: 6 - protein, 356 aa, Artificial Sequence
  source: /note=“Description of Artificial Sequence Synthetic polypeptide”
MPLLLLLPLLWAGALAQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEGSHHHHHH

SEQ ID NO: 7 - protein, 591 aa, Artificial Sequence
  source: /note=“Description of Artificial Sequence Synthetic polypeptide”
MPLLLLLPLLWAGALAQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEGSRIPKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCRVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 866 aa, Artificial Sequence
  source: /note=“Description of Artificial Sequence Synthetic polypeptide”
MPLLLLLPLLWAGALALYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRGSRIPKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCRVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 394 aa, Homo sapiens
MIVFIFLAMGLSLENEYTSQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSSIQIPGEL

SEQ ID NO: 10 - protein, 16 aa, Homo sapiens
MPLLLLLPLLWAGALA

SEQ ID NO: 11 - protein, 138 aa, Artificial Sequence
  source: /note=“Description of Artificial Sequence Synthetic polypeptide”
MPLLLLLPLLWAGALAHHHHHHGSGGARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 12 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 13 - protein, 112 aa, Homo sapiens
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 14 - protein, 109 aa, Homo sapiens
GDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 15 - protein, 112 aa, Artificial Sequence
  source: /note=“Description of Artificial Sequence Synthetic polypeptide”
AHNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 16 - protein, 112 aa, Artificial Sequence
  source: /note=“Description of Artificial Sequence Synthetic polypeptide”
AHAGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 17 - protein, 112 aa, Artificial Sequence
  source: /note=“Description of Artificial Sequence Synthetic polypeptide”
AREGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 18 - protein, 20 aa, Homo sapiens
VIYAAMCMTVTCGILLLVMV

SEQ ID NO: 19 - protein, 32 aa, Artificial Sequence
  source: /note=“Description of Artificial Sequence Synthetic polypeptide”
GSGTTSGTTRLLSSHTSAALTVLSVLMLKLAL

SEQ ID NO: 20 - protein, 24 aa, Artificial Sequence
  source: /note=“Description of Artificial Sequence Synthetic peptide”
SGPSRARPSAALTVLSVLMLKLAL

SEQ ID NO: 21 - protein, 34 aa, Artificial Sequence
  source: /note=“Description of Artificial Sequence Synthetic polypeptide”
GSGTTSGTTRLLSGHTCFTLTGLLGTLVTMGLLT

SEQ ID NO: 22 - protein, 31 aa, Artificial Sequence
  source: /note=“Description of Artificial Sequence Synthetic polypeptide”
DSSSSWWTNWVIPAISAVAVALMYRLYMAED

SEQ ID NO: 23 - protein, 31 aa, Artificial Sequence
  source: /note=“Description of Artificial Sequence Synthetic polypeptide”
ESDSSWWTNWVIPAISALVVALMYRLYMAED

SEQ ID NO: 24 - DNA, 1911 bp, Homo sapiens
ttattctggacagttactcttaagaaagttgtcagaagaaacgcatctgcctttttttccaggtgaactgccgtgagttgtccagcatgatagtgtttattttcttggctatggggttaagcttggaaaatgaatacacttcccaaaccaataattgcacatatttaagagagcaatgcttacgtgatgcaaatggatgtaaacatgcttggagagtaatggaagatgcctgcaatgattcagatccaggtgacccctgcaagatgaggaattcatcatactgtaacctgagtatccagtacttagtggaaagcaatttccaatttaaagagtgtctttgcactgatgacttctattgtactgtgaacaaactgcttggaaaaaaatgtatcaataaatcagataacgtgaaagaggataaattcaaatggaatctaactacacgttcccatcatggattcaaagggatgtggtcctgtttggaagtggcagaggcatgtgtaggggatgtggtctgtaatgcacagttggcctcttaccttaaagcttgctcagcaaatggaaatccgtgtgatctgaaacagtgccaagcagccatacggttcttctatcaaaatataccttttaacattgcccagatgttggctttttgtgactgtgctcaatctgatataccttgtcagcagtccaaagaagctcttcacagcaagacatgtgcagtgaacatggttccaccccctacttgcctcagtgtaattcgcagctgccaaaatgatgaattatgcaggaggcactatagaacatttcagtcaaaatgctggcagcgtgtgactagaaagtgccatgaagatgagaattgcattagcaccttaagcaaacaggacctcacttgttcaggaagtgatgactgcaaagctgcttacatagatatccttgggacggtccttcaagtgcaatgtacctgtaggaccattacacaaagtgaggaatctttgtgtaagattttccagcacatgcttcatagaaaatcatgtttcaattatccaaccctgtctaatgtcaaaggcatggcattgtatacaagaaaacatgcaaacaaaatcactttaactggatttcattcccccttcaatggagaagtaatctatgctgccatgtgcatgacagtcacctgtggaatccttctgttggttatggtcaagcttagaacttccagaatatcaagtaaagcaagagatccttcatcgatccaaatacctggagaactctgattcattaggagtcatggacctataacaatcactcttttctctgcttttcttctttcctcttttcttctctcctctcctctcctctcttctcctctcctcccctcccctctctgtttctttttctttttcttttcttttttgtggcggagttttgctcttgttgcccaggctgcagtacaatggctcaatctcggttcactgcaacctctgcctccaaggttcaagtgattttcctgcctcagccttcccgagtagctgggattacaggtacccgccaccacgcccagctaatttttttgtatttttagtagagatggggttttgccaaattggccagggtggtctcaaactcctgacctcaggtgatccacccacctcggcctcccaaagtgctgggattacaggcgtgagcaaccacgtcaagacaacaatcactttctttaaagcaaatcctacagctggtcaacaccctattccatctgtcatcgagaaagaaaatgttaaaatagacttaaaaatattgctttgttacatataataatatggcatgatgatgttatttttttcttaatactcaagaaaaaatatatggtggtatcttttacaacactggaacagaaataaagtttcccttgaaggc

SEQ ID NO: 25 - protein, 235 aa, Artificial Sequence
  source: /note=“Description of Artificial Sequence Synthetic polypeptide”
MPLLLLLPLLWAGALAGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKGSHHHHHH

SEQ ID NO: 26 - protein, 6 aa, Artificial Sequence
  source: /note=“Description of Artificial Sequence Synthetic 6xHis tag”
HHHHHH